(19) United States
(12) Patent Application Publication
(10) Pub. No.: US 2026/0096902 A1
(43) Pub. Date: Apr. 9, 2026

(54) INTERVERTEBRAL DISC PROSTHESIS, AND SYSTEMS AND METHODS FOR IMPLANTATION OF THE SAME

(71) Applicant: Highridge Medical, LLC, Westminster, CO (US)

(72) Inventors: Nicolas Roche, Saint Medard En Jalles (FR); Nicolas Bidegaimberry, Gradignan (FR); David Rigotto, Saint Selve (FR)

(21) Appl. No.: 19/315,272

(22) Filed: Aug. 29, 2025

Related U.S. Application Data
(60) Provisional application No. 63/703,686, filed on Oct. 4, 2024.
(60) Provisional application No. 63/703,700, filed on Oct. 4, 2024.
(60) Provisional application No. 63/703,708, filed on Oct. 4, 2024.

(51) Int. Cl. A61F 2/44; A61F 2/30

(52) U.S. Cl. CPC A61F 2/4425; A61F 2002/30115; A61F 2002/30245; A61F 2002/30329; A61F 2002/30843; A61F 2002/443

(57) ABSTRACT

Systems and methods for implantation of an intervertebral disc prosthesis include the prosthesis, a cartridge, and an instrument. Prior to implantation of the prosthesis, the cartridge receives and engages with the prosthesis to form an assembly. In addition, the instrument receives the assembly within an opening in an outer housing and engages the cartridge with a coupler of a locking shaft. Following implantation of the cartridge, actuation of a release driver causes an expansion bit of the release driver to engage release tabs on the cartridge to cause the cartridge to disengage from the prosthesis. The systems and methods optionally include a trial implant and/or an osteotome for trialing prior to implantation of the prosthesis.

### CROSS-REFERENCE TO RELATED APPLICATIONS

This U.S. Non-Provisional Patent Application claims the benefit of priority under 35 U.S.C. § 119(e) of U.S. Provisional Patent Application Ser. No. 63/703,686, filed Oct. 4, 2024; U.S. Provisional Patent Application Ser. No. 63/703,700, filed Oct. 4, 2024; and U.S. Provisional Patent Application Ser. No. 63/703,708, filed Oct. 4, 2024, the entire disclosures of each of which are hereby incorporated by reference.

### FIELD OF THE DISCLOSURE

The disclosure relates to an intervertebral disc prosthesis and, in particular, is directed to systems and methods for implantation of the intervertebral disc prosthesis using a cartridge and an instrument.

### BACKGROUND

The background description includes information that may be useful in understanding the present inventive subject matter. It is not an admission that any of the information provided herein is prior art or relevant to the presently claimed disclosure, or that any publication specifically or implicitly referenced is prior art.

Various types of intervertebral disc prostheses are known in the prior art. The known prostheses are installed within a cavity defined (e.g., during a discectomy) between adjacent vertebral bodies using an implantation tool. Recent attempts have been made to reduce the invasiveness of the operation by reducing the size of the implantation site needed during implantation of the prostheses.

### SUMMARY

An intervertebral disc prosthesis includes an upper plate, a lower plate, and a central core or insert therebetween. The central core or insert may be able to slide within a cavity defined between the upper plate and the lower plate. The movement of the insert relative to the upper plate and/or the lower plate allows for spontaneous positioning of the central core or insert in the ideal position, including to absorb constraints imposed on the prosthesis during movements made by the patient in which the prosthesis is implanted.

However, because of the forces from applied constraints, materials utilized, and the form of the different components from which the prosthesis is assembled, the sliding of the insert can cause rapid wear and heightens the risk of ejection of at least a part of the prosthesis to be exterior to the spine. In addition, positioning of the prosthesis within the spine (e.g., between adjacent vertebral bodies, in the case of a prosthesis with the upper plate, the lower plate, and the insert) requires increased precision and accuracy as efforts are made to further reduce the invasive nature of the operation during implantation, while also promoting the correct placement and retaining of the prosthesis within the spine (e.g., in the cavity defined between the adjacent vertebral bodies).

As such, there exists a need for an improved intervertebral disc prosthesis. Embodiments of the present disclosure are directed to an intervertebral disc prosthesis with an upper plate (or superior plate), a lower plate (or inferior plate), and an insert (or central core) enclosed therebetween. The upper plate has a superior surface and the lower plate has an inferior surface that each contact a respective surface of the adjacent vertebral bodies defining a cavity within which the prosthesis is implanted.

In embodiments, the intervertebral disc prosthesis includes teeth or other protrusions that extend from the respective contacting surfaces of the upper plate and the lower plate. The engagement of the teeth with the adjacent vertebral bodies reduces the possibility of slippage or other movement of the prosthesis within the cavity defined between the adjacent vertebral bodies. For example, the teeth may be approximately even in height relative to the respective contact surface from which the teeth originate. By way of another example, at least some of the teeth may be different in height relative to the respective contacting surface from which the teeth originate, as compared to other teeth on the same contacting surface.

Optionally, the teeth are in one or more sets or groupings. The one or more sets may flank a midline defined through the front and rear ends of the respective upper and lower plates. The one or more sets may be positioned on the midline itself. For example, a set of teeth positioned on the midline may be used for sighting of the prosthesis during implantation, including in combination with features on implantation tools used during implantation of the prosthesis, and/or for monitoring the position of the prosthesis after implantation.

In embodiments, the insert of the intervertebral disc prosthesis includes a superior surface that interfaces with an inferior surface of the upper plate, and an inferior surface that interfaces with a superior surface of the lower plate. The insert is able to reposition within the cavity or space defined between the inferior surface of the upper plate and the superior surface of the lower plate, allowing for adjustment during movement by the patient in which the prosthesis is implanted.

Optionally, the superior surface of the insert is at least partially dome-shaped (e.g., is a semi-spherical convex surface, or the like), and mates with a corresponding dome-shaped recess (e.g., is a semi-spherical concave surface, or the like) in the inferior surface of the upper plate. In some instances, the dome-shaped feature on the superior surface of the insert may extend to a sidewall of the insert. In other instances, the dome-shaped feature on the superior surface of the insert may be at least partially surrounded by a second portion of the superior surface of the insert, such as an annular ring that is substantially flat or planar. In some configurations, the annular ring is configured to mate with a rim on the inferior surface of the upper plate in select orientations of the upper plate relative to the insert, which may serve to limit or constrain movement of the upper plate at outer boundaries of the range for the angle of motion of the upper plate.

Optionally, the second surface of the insert includes grooves or cutouts to promote repositioning. For example, a cutout in the second surface of the insert may receive a protrusion on the superior surface of the lower plate. In some instances, the dimensions of the cutout are increased in size relative to the dimensions of the protrusion to allow the repositioning of the insert relative to the lower plate, while still allowing the protrusion to hold the insert within the cavity or space defined between the upper and lower plates.

In some embodiments, a standard positioning or natural state of the prosthesis includes the upper plate being misaligned with the lower plate. For example, the upper plate may have a central axis through the respective superior and inferior surfaces that is offset from (e.g., slightly forward of) and angled with respect to (e.g., downward toward) a central axis through the respective superior and inferior surfaces of the lower plate.

There is also a need for improved systems and methods for implantation of the intervertebral disc prosthesis. Embodiments of the present disclosure are directed to an instrument for implantation of the prosthesis. In some embodiments, the instrument interfaces with the prosthesis via a cartridge during implantation. In additional embodiments, the instrument interfaces with trialing components such as a trial implant and/or an osteotome.

The cartridge includes one or more arms that engage with the prosthesis at engagement points on the prosthesis. For example, a rear or distal end of the upper plate and/or the lower plate of the prosthesis may include engagement features (e.g., shelves, notches, or the like) to corresponding engagement features (e.g., ledges, tabs, or the like) on the proximal ends of the arms of the cartridge may engage. These corresponding engagement features promote a more secure connection between the cartridge and the prosthesis (e.g., such as during implantation). In addition, the corresponding engagement features promote the correct positioning of the prosthesis between the adjacent vertebral bodies (e.g., such as during implantation), including with the optional misalignment positioning of the upper plate with respect to the lower plate. In some embodiments, a particular arm of the cartridge may include a protrusion that interfaces with (e.g., is inserted into) the cavity or space defined between the upper and lower plates of the prosthesis, to further promote the connection between the cartridge and the prosthesis.

In embodiments, surfaces of the prosthesis and the cartridge are substantially flush or planar when the cartridge is connected to the prosthesis. For example, a superior surface, an inferior surface, and/or at least one side surface of the arms of the cartridge may be substantially flush or planar with a corresponding surface on the prosthesis, such that the cartridge does not exceed the height and/or the width of the prosthesis at the connection. The substantially flush or planar nature of the one or more corresponding surfaces allows for the reduction of the dimensions of the implantation site.

In embodiments, the instrument includes an outer housing, a locking shaft, and a release driver. Each of the outer housing, the locking shaft, and the release driver is actuatable via a respective actuator. One or more of the actuation of the outer housing, the locking shaft, and the release driver may be monitored via markers on the instrument (e.g., on the outer housing), with the actuator moving an indicator with respect to the markers.

For example, the outer housing has depth stops that are positionable relative to a cartridge and prosthesis assembly inserted into an opening in the outer housing, via actuation of a first actuator (e.g., a knob or lever) at a distal end of the instrument. For instance, the outer housing is translatable following a rotation of the first actuator.

By way of another example, the locking shaft is actuatable via a second actuator (e.g., a knob or lever) at a distal end of the instrument. For instance, the locking shaft is rotatable within the outer housing following a rotation of the second actuator, to cause the locking shaft to engage the cartridge or a trial implant inserted within the instrument.

By way of another example, the release driver is actuatable via a third actuator (e.g., a knob or lever) at a distal end of the instrument. For instance, the release driver is rotatable within the locking shaft following a rotation of the third actuator, which causes a rotation or translation of an expansion bit. For instance, the release driver may have a first portion and a second portion, where the second portion is a locking pin, and the expansion bit may be a component of the locking pin. In some aspects, the locking pin may be considered a component of the cartridge (or a component of an assembly including the cartridge), where the locking pin is couplable to the first portion of the release driver following insertion of the cartridge into the instrument.

The instrument interfaces with the cartridge at a proximal end of the instrument and a distal end of the cartridge. For example, the cartridge (or cartridge and prosthesis assembly) is inserted into an opening within the outer housing at the proximal end of the instrument, while the locking shaft is in a first orientation. Actuation of the locking shaft causes a coupler at a proximal end of the locking shaft to transition to a second orientation, where the cartridge is held within the proximal end of the instrument. In some configurations, the locking pin of the release driver is provided with the cartridge and prosthesis assembly, and the locking shaft receives a distal end of the locking pin to allow for engagement between the locking pin and a second portion of the release driver.

The instrument is further configured to engage with the cartridge (e.g., once the cartridge and prosthesis assembly is inserted into the opening). For example, once the cartridge is secured within the outer housing via the proximal end of the locking shaft being in the second orientation, the release driver may be actuated to cause the arms of the cartridge to expand outward from a first position (e.g., in which the prosthesis is engaged by the cartridge) to a second position (e.g., in which the prosthesis is released by the cartridge). In this regard, securing the cartridge to the outer housing via the locking shaft may be used to implant the prosthesis between the adjacent vertebral bodies and the release driver may then be actuated to cause the cartridge to disengage from the implanted prosthesis, allowing for the removal of the instrument with cartridge from the implantation site after the prosthesis is deposited.

In embodiments, the release driver includes the first portion with the third actuator that is removable from the instrument, and the second portion or locking pin with the expansion bit. The second portion or locking pin with the expansion bit may be included in the cartridge and prosthesis assembly (or may be considered a component of the cartridge), such that the first portion with the third actuator of the release driver is inserted into the instrument and engages with the second portion or locking pin after insertion of the assembly into the opening of the instrument. It is noted, however, that the release driver may be a single integrated component extending from the third actuator to the expansion bit of the locking pin without departing from the scope of the present disclosure.

In embodiments, the instrument has a locking mechanism to prevent actuation of the outer housing, the locking shaft, and/or the release driver. In one non-limiting example, the locking mechanism may prevent actuation of the outer housing and/or the locking shaft while the release driver is being actuated, to prevent accidental adjustment of the depth stops and/or accidental release of the cartridge from the instrument while implanting the prosthesis.

In embodiments, the cartridge and prosthesis are insertable into a cavity defined between adjacent vertebral bodies with an instrument. Known implantation systems require leaving the cartridge and prosthesis in the implantation site after implantation of the prosthesis and removal of the instrument, and then engaging with the cartridge with a different instrument (e.g., pliers, forceps, or the like) to remove the cartridge from the implantation site. In contrast, the present invention is directed to a cartridge and prosthesis assembly that may be acted upon by one instrument, where the one instrument is capable of accepting the cartridge and prosthesis assembly, implanting the prosthesis, and actuating the cartridge to cause the cartridge to disengage from the prosthesis and subsequently be removed along with the instrument.

Because the cartridge remains within the instrument, the cartridge may not require a pivot pin or other means of securing the arms of the cartridge together. This reduces the complexity of the cartridge, as additional components like hinge portions, pivot pins, biasing elements, and the like may be removed from the cartridge. However, it is contemplated that a pivoting mechanism and corresponding components may be included on the cartridge, without departing from the scope of the present disclosure.

Further, embodiments of the present disclosure are directed to a trial implant and an osteotome for use to assist in the sizing of the implantation site and the selection of the prosthesis. In some embodiments, the instrument interfaces with the trial implant in place of the cartridge and prosthesis assembly. For example, a body of the trial implant may be inserted into the opening at the proximal end of the outer housing while the locking shaft is in a first orientation. Actuation of the locking shaft causes the coupler at the proximal end of the locking shaft to transition to a second orientation, where the cartridge is held within the proximal end of the instrument.

In some embodiments, the trial implant is a static size having a head with a pre-defined depth, width, and/or height. In other embodiments, the trial implant is adjustable in one or more of depth, width, and/or height, including via actuation of the release driver following the securing of the trial implant within the opening of the outer housing via the locking shaft. In general, the trial implant has a body and a head that is configured to approximate the dimensions of the cartridge and a particular prosthesis having a pre-defined depth, width, and/or height, so that the body interfaces with the instrument in place of the cartridge and the head sizes the cavity defined between adjacent vertebral bodies for the particular prosthesis.

In embodiments, the instrument includes an osteotome. The osteotome is used to mark a midline on the vertebral body during trialing with the trial implant. Optionally, the trial implant includes a midline on the head that aligns with the midline marked on the vertebral body by the osteotome.

A first aspect of the present disclosure is to provide an intervertebral disc prosthesis system. The system comprises an instrument including an outer housing with an opening at a proximal end of the outer housing, a locking shaft with a coupler at a proximal end of the locking shaft, and a release driver with an expansion bit at a proximal end of the release driver. The system comprises a cartridge with release tabs and a receiver defined at a proximal end of the cartridge. The system comprises a prosthesis having a distal end that is insertable into the receiver to form an assembly with the cartridge. A distal end of the cartridge is insertable into the opening at the proximal end of the instrument. The locking shaft is actuatable between a first orientation and a second orientation, wherein the coupler of the locking shaft is insertable into the distal end of the cartridge when the locking shaft is in the first orientation, wherein the coupler of the locking shaft is retained within the distal end of the cartridge when the locking shaft is in the second orientation. Actuation of the release driver causes the expansion bit to engage the release tabs to release the prosthesis from the receiver of the cartridge.

The system of the first aspect may include, optionally, an osteotome insertable into a channel on the outer housing; and a trial implant insertable into the opening at the proximal end of the outer housing when the cartridge is not inserted into the opening.

A second aspect of the present disclosure is to provide a prosthesis for an intervertebral disc prosthesis system. The prosthesis comprises an upper plate having a superior surface with teeth and an inferior surface, a lower plate having a superior surface with a protrusion and an inferior surface with teeth, and an insert having a superior surface that is dome-shaped and a substantially flat or planar inferior surface with a recess able to receive the protrusion, the insert being positioned between the upper plate and the lower plate. At least one of a rear edge of the upper plate or a rear edge of the lower plate includes at least one engagement feature to couple the prosthesis to a cartridge insertable into an instrument.

The system of the second aspect may include, optionally, that the upper plate includes a dome region that is contoured to conform to the dome-shaped superior surface of the insert.

A third aspect of the present disclosure is to provide a cartridge for an intervertebral disc prosthesis system. The cartridge comprises a first arm having a first hand at a first proximal end and a first tail at a first distal end; and a second arm having a second hand at a second proximal end and a second tail at a second distal end. The first hand and the second hand define a receiver with at least one engagement feature to couple the cartridge to a prosthesis. The first tail and the second tail a slot and a cavity. The first tail and the second tail are insertable into an opening of an instrument. The instrument is able to engage the slot and the cavity to retain the cartridge within the instrument when the first tail and the second tail are inserted into the opening. The instrument is able to engage a release tab on at least one of the first arm and the second arm to cause the receiver to release the prosthesis.

The cartridge of the third aspect may include, optionally, a locking pin of a release driver of the instrument, wherein the locking pin is inserted within the first arm and the second arm, and wherein the locking pin is able to engage the release tab on at least one of the first arm and the second arm to cause the receiver to release the prosthesis.

The cartridge of the third aspect may include one or more of the previous embodiments and, optionally, that the locking pin is a portion of the release driver that is provided with the cartridge, and wherein the locking pin is able to engage with and be rotated by an actuator portion of the release driver.

A fourth aspect of the present disclosure is to provide an instrument for an intervertebral disc prosthesis system. The instrument comprises an outer housing with an opening at a proximal end of the outer housing, wherein the outer housing is actuatable to set a position of depth stops on the outer housing. The instrument comprises a locking shaft with a coupler at a proximal end of the locking shaft, wherein a coupler of the locking shaft is actuatable to engage an assembly including a cartridge and a prosthesis when the assembly including the cartridge and the prosthesis is inserted into the opening of the outer housing. The instrument comprises a release driver with an expansion bit, wherein the release driver is actuatable to cause an expansion bit of the release driver to engage release tabs on the cartridge to release the prosthesis from the cartridge when the assembly including the cartridge and the prosthesis is inserted into the opening of the outer housing.

The instrument of the fourth aspect may include, optionally, that the coupler of the locking shaft is actuatable to engage a trial implant when the trial implant is inserted into the opening of the outer housing and the assembly including the cartridge and the prosthesis is not inserted into the opening of the outer housing.

The instrument of the fourth aspect may include one or more of the previous embodiments and, optionally, that the depth stops define a channel for an osteotome for use during trialing with a trial implant, when the trial implant is inserted into the opening of the outer housing.

The instrument of the fourth aspect may include one or more of the previous embodiments and, optionally, wherein the outer housing is actuatable via a first actuator, the coupler of the locking shaft is actuatable via a second actuator, and the release driver is actuatable via a third actuator.

The instrument of the fourth aspect may include one or more of the previous embodiments and, optionally, that the release driver includes a first portion with the third actuator and a locking pin with the expansion bit that is provided with the assembly including the cartridge and the prosthesis when the assembly including the cartridge and the prosthesis is inserted into the opening, and wherein the first portion engages with the locking pin when the assembly including the cartridge and the prosthesis is inserted into the opening.

A fifth aspect of the present disclosure is to provide a method for implantation of an intervertebral disc prosthesis. The method may include, but is not limited to, coupling a prosthesis to a cartridge via at least one engagement feature to form an assembly. The method may include, but is not limited to, inserting the assembly into an opening in an outer housing of an instrument and engaging the cartridge with a coupler of a locking shaft of the instrument. The method may include, but is not limited to, releasing the prosthesis from the cartridge following implantation of the prosthesis by engaging release tabs of the cartridge with an expansion bit of a release driver of the instrument.

The method of the fifth aspect may include, optionally, prior to inserting the assembly into the opening in the outer housing, inserting a trial implant into the opening in the outer housing of the instrument. The method may include, optionally, prior to inserting the assembly into the opening in the outer housing, marking a midline for the implantation of the prosthesis with an osteotome set within a channel defined by at least one depth stop on the outer housing.

The method of the fifth aspect may include one or more of the previous embodiments and, optionally, prior to marking the midline, adjusting a depth of the at least one depth stop on the outer housing.

The method of the fifth aspect may include one or more of the previous embodiments and, optionally, that the assembly further includes a locking pin of the release driver. The locking pin is able to engage with an actuator portion of the release driver. The expansion bit is a component of the locking pin.

A sixth aspect of the present disclosure is directed to a prosthesis for an intervertebral disc prosthesis system. The prosthesis comprises an upper plate including a superior surface with at least one set of upper teeth, and an inferior surface. The prosthesis comprises a lower plate including a superior surface with at least one protrusion, and an inferior surface with at least one set of lower teeth. The prosthesis comprises an insert located between the upper plate and the lower plate, wherein the insert includes a superior surface with a convex region, wherein the insert includes an inferior surface that is substantially flat, wherein the insert includes at least one recess able to receive the at least one protrusion, wherein a particular recess of the at least one recess corresponds to a particular protrusion of the at least one protrusion. At least one of a rear edge of the upper plate and a rear edge of the lower plate includes at least one engagement feature able to engage with a corresponding engagement feature of a cartridge that is insertable into an instrument.

The prosthesis of the sixth aspect may include, optionally, that the at least one set of upper teeth of the upper plate includes a first set of upper teeth proximate to a first side edge of the upper plate, and a second set of upper teeth proximate to a second side edge of the upper plate.

The prosthesis of the sixth aspect may include one or more of the previous embodiments and, optionally, that the at least one engagement feature is on the upper plate, the first set of upper teeth of the upper plate at least partially align with a first engagement feature of the at least one engagement feature on the upper plate, and the second set of upper teeth of the upper plate at least partially align with a second engagement feature of the at least one engagement feature on the upper plate.

The prosthesis of the sixth aspect may include one or more of the previous embodiments and, optionally, that the upper plate includes one or more alignment teeth located along a midline of the upper plate.

The prosthesis of the sixth aspect may include one or more of the previous embodiments and, optionally, that the one or more alignment teeth are located on a convex region of the superior surface of the upper plate, and the at least one set of upper teeth includes a first set of upper teeth and a second set of upper teeth that flank the convex region of the superior surface of the upper plate.

The prosthesis of the sixth aspect may include one or more of the previous embodiments and, optionally, that the inferior surface of the upper plate includes a concave portion that is contoured to conform to the convex region of the superior surface of the insert.

The prosthesis of the sixth aspect may include one or more of the previous embodiments and, optionally, that the concave portion of the inferior surface of the upper plate is at least partially surrounded by a substantially flat portion of the inferior surface of the upper plate.

The prosthesis of the sixth aspect may include one or more of the previous embodiments and, optionally, that the concave portion of the inferior surface of the upper plate is separated from the substantially flat portion of the inferior surface of the upper plate by a rim that extends from the inferior surface.

The prosthesis of the sixth aspect may include one or more of the previous embodiments and, optionally, that the convex region of the superior surface of the insert is at least partially surrounded by a ring, and the ring is able to engage with the rim extending from the inferior surface of the upper plate.

The prosthesis of the sixth aspect may include one or more of the previous embodiments and, optionally, that the at least one set of lower teeth of the lower plate includes a first set of lower teeth proximate to a first side edge of the lower plate, and a second set of lower teeth proximate to a second side edge of the lower plate.

The prosthesis of the sixth aspect may include one or more of the previous embodiments and, optionally, that the at least one engagement feature is on the lower plate, the first set of lower teeth of the lower plate is located between a midline of the lower plate and a first engagement feature of the at least one engagement feature on the lower plate, and the second set of lower teeth of the lower plate is positioned between the midline of the lower plate and a second engagement feature of the at least one engagement feature on the lower plate.

The prosthesis of the sixth aspect may include one or more of the previous embodiments and, optionally, that the lower plate includes one or more alignment teeth located along a midline of the lower plate.

The prosthesis of the sixth aspect may include one or more of the previous embodiments and, optionally, that the at least one protrusion includes a single protrusion located on a midline of the lower plate, and the at least one recess includes a single recess that is located on a midline of the insert and extends a selected depth into the inferior surface of the insert.

The prosthesis of the sixth aspect may include one or more of the previous embodiments and, optionally, that the at least one protrusion includes a first protrusion located proximate to a first side edge of the lower plate, and a second protrusion located proximate to a second side edge of the lower plate. The at least one recess includes a first recess corresponding to the first protrusion and that intersects a first sidewall of the insert while extending through the insert from the inferior surface of the insert to the superior surface of the insert, and a second recess corresponding to the second protrusion and that intersects a second sidewall of the insert while extending through the insert from the inferior surface of the insert to the superior surface of the insert.

The prosthesis of the sixth aspect may include one or more of the previous embodiments and, optionally, that the at least one engagement feature is on the upper plate, and the at least one engagement feature includes a ledge that extends from the rear edge of the upper plate to a notch in the upper plate.

The prosthesis of the sixth aspect may include one or more of the previous embodiments and, optionally, that the ledge transitions directly into the notch such that a shared surface of the ledge and the notch is at least partially defined by an edge with a straight portion.

The prosthesis of the sixth aspect may include one or more of the previous embodiments and, optionally, that the at least one engagement feature is on the lower plate, and the at least one engagement feature includes a ledge that extends from the rear edge of the lower plate to a notch in the lower plate.

The prosthesis of the sixth aspect may include one or more of the previous embodiments and, optionally, that the ledge transitions directly into the notch such that a shared surface of the ledge and the notch is at least partially defined by an edge with a straight portion.

A seventh aspect of the present disclosure is directed to a prosthesis for an intervertebral disc prosthesis system. The prosthesis comprises an upper plate including a superior surface with at least one set of upper teeth, and an inferior surface. The prosthesis comprises a lower plate including a superior surface with a first protrusion located proximate to a first side edge of the lower plate and a second protrusion located proximate to a second side edge of the lower plate, and an inferior surface with at least one set of lower teeth. The prosthesis comprises an insert located between the upper plate and the lower plate, wherein the insert includes a superior surface with a convex region, wherein the insert includes an inferior surface that is substantially flat, wherein the insert includes a first recess corresponding to the first protrusion and that intersects a first sidewall of the insert while extending through the insert from the inferior surface of the insert to the superior surface of the insert, wherein the insert includes a second recess corresponding to the second protrusion and that intersects a second sidewall of the insert while extending through the insert from the inferior surface of the insert to the superior surface of the insert. At least one of a rear edge of the upper plate and a rear edge of the lower plate includes at least one engagement feature able to engage with a corresponding engagement feature of a cartridge insertable into an instrument of the intervertebral disc prosthesis system.

An eighth aspect of the present disclosure is directed to a prosthesis for an intervertebral disc prosthesis system. The prosthesis comprises an upper plate including a superior surface with at least one set of upper teeth, and an inferior surface, wherein the upper plate includes at least one engagement feature with a ledge that extends from a rear edge of the upper plate to a notch in the upper plate, wherein the ledge of the upper plate transitions directly into the notch of the upper plate such that a shared surface of the ledge and the notch of the upper plate is at least partially defined by an edge with a straight portion of the upper plate. The prosthesis comprises a lower plate including a superior surface with at least one protrusion, and an inferior surface with at least one set of lower teeth, wherein the lower plate includes at least one engagement feature with a ledge that extends from a rear edge of the lower plate to a notch in the lower plate, wherein the ledge of the lower plate transitions directly into the notch of the lower plate such that a shared surface of the ledge and the notch of the lower plate is at least partially defined by an edge with a straight portion of the lower plate. The prosthesis comprises an insert located between the upper plate and the lower plate, wherein the insert includes a superior surface with a convex region, wherein the insert includes an inferior surface that is substantially flat, wherein the insert includes at least one recess able to receive the protrusion, wherein a particular recess of the at least one recess corresponds to a particular protrusion of the at least one protrusion.

A ninth aspect of the present disclosure is directed to a cartridge for an intervertebral disc prosthesis system. The cartridge comprises a first arm having a first hand at a proximal end of the cartridge and a first tail at a distal end of the cartridge. The cartridge comprises a second arm having a second hand at the proximal end of the cartridge and a second tail at the distal end of the cartridge. The first hand and the second hand define a receiver with at least one engagement feature able to engage with a corresponding engagement feature of a prosthesis. The first tail and the second tail are insertable into an opening of an instrument, wherein the first tail and the second tail define a slot and a cavity dimensioned to mate with a coupler of a locking shaft of the instrument. At least one of the first arm and the second arm includes a release tab that is able to engage a locking pin of a release driver of the instrument, and wherein engagement of the release tab with the locking pin causes the receiver to release the prosthesis.

The cartridge of the ninth aspect may include, optionally, that the first arm includes a first portion proximate to the first hand at the proximal end of the cartridge, the second arm includes a second portion proximate to the second hand at the proximal end of the cartridge, the first portion and the second portion define an end cavity dimensioned to receive an expansion bit of the locking pin of the release driver of the instrument, and the expansion bit of the locking pin being positioned within the end cavity promotes the prosthesis remaining secured within the cartridge prior to engagement of the release tab with the locking pin to release the prosthesis.

The cartridge of the ninth aspect may include one or more of the previous embodiments and, optionally, that the first arm includes a first crossmember with a first release tab, the second arm includes a second crossmember with a second release tab, and the first release tab and the second release tab are dimensioned to receive a middle portion of the locking pin of the release driver of the instrument.

The cartridge of the ninth aspect may include one or more of the previous embodiments and, optionally, that a width between the first release tab and the second release tab is less than a width of the end cavity defined within the proximal end of the cartridge, such that the first arm and the second arm are configured to expand outward when the expansion bit of the locking pin makes contact with the first release tab and the second release tab.

The cartridge of the ninth aspect may include one or more of the previous embodiments and, optionally, that the first arm includes a first crossmember at the distal end of the cartridge, the second arm includes a second crossmember at the distal end of the cartridge, and the first crossmember and the second crossmember define an opening dimensioned to receive a distal portion of the locking pin of the release driver of the instrument.

The cartridge of the ninth aspect may include one or more of the previous embodiments and, optionally, that the slot and the cavity are able to receive the coupler of the instrument when the first tail and the second tail are inserted into the opening of the instrument and when the coupler is in a first orientation, and the cavity mates with the coupler of the instrument to retain the cartridge within the opening of the instrument when the coupler is in a second orientation.

The cartridge of the ninth aspect may include one or more of the previous embodiments and, optionally, that the at least one engagement feature of the receiver includes a shelf positionable proximate to a ledge of a plate of the prosthesis and a tab positionable within a notch of the plate of the prosthesis.

The cartridge of the ninth aspect may include one or more of the previous embodiments and, optionally, that the ledge transitions directly into the notch of the prosthesis such that a shared surface of the ledge and the notch of the prosthesis is at least partially defined by an edge with a straight portion, and the receiver includes a straight portion that corresponds to the straight portion of the edge of the shared surface of the prosthesis.

The cartridge of the ninth aspect may include one or more of the previous embodiments and, optionally, that the at least one engagement feature includes an engagement feature on the first arm, which is able to engage a corresponding engagement feature on an upper plate of the prosthesis.

The cartridge of the ninth aspect may include one or more of the previous embodiments and, optionally, that the at least one engagement feature includes an engagement feature on the first arm, which is able to engage a corresponding engagement feature on a lower plate of the prosthesis.

The cartridge of the ninth aspect may include one or more of the previous embodiments and, optionally, that the at least one engagement feature includes an engagement feature on the second arm, which is able to engage a corresponding engagement feature on an upper plate of the prosthesis.

The cartridge of the ninth aspect may include one or more of the previous embodiments and, optionally, that the at least one engagement feature includes an engagement feature on the second arm, which is able to engage a corresponding engagement feature on a lower plate of the prosthesis.

The cartridge of the ninth aspect may include one or more of the previous embodiments and, optionally, that the first arm and the second arm each include a superior surface that is substantially planar with at least a portion of a superior surface of an upper plate of the prosthesis when the cartridge engages with the prosthesis.

The cartridge of the ninth aspect may include one or more of the previous embodiments and, optionally, that the first arm and the second arm each include an inferior surface that is substantially planar with at least a portion of an inferior surface of a lower plate of the prosthesis when the cartridge engages with the prosthesis.

The cartridge of the ninth aspect may include one or more of the previous embodiments and, optionally, that the first arm includes a first finger with a first exterior sidewall that is substantially planar with at least a portion of a first side surface of an upper plate of the prosthesis and at least a portion of a first side surface of a lower plate of the prosthesis when the cartridge engages with the prosthesis. The second arm includes a second finger with a second exterior sidewall that is substantially planar with at least a portion of a second side surface of the upper plate of the prosthesis and at least a portion of a second side surface of the lower plate of the prosthesis when the cartridge engages with the prosthesis.

The cartridge of the ninth aspect may include one or more of the previous embodiments and, optionally, that at least one of the first arm and the second arm includes a locking tab that is able to engage a corresponding locking recess on the locking pin of the release driver.

The cartridge of the ninth aspect may include one or more of the previous embodiments and, optionally, that at least one of the first arm and the second arm includes a wedge that is able to engage with a corresponding tab on the instrument, to assist in aligning the cartridge during insertion of the cartridge into the opening of the instrument.

The cartridge of the ninth aspect may include one or more of the previous embodiments and, optionally, that the cartridge, the prosthesis, and the locking pin are packaged as an assembly. The locking pin couples to a second portion of the release driver of the instrument when the cartridge is inserted into the opening of the instrument.

A tenth aspect of the present disclosure is directed to a cartridge for an intervertebral disc prosthesis system. The cartridge comprises a first arm having a first hand at a proximal end of the cartridge and a first tail at a distal end of the cartridge. The cartridge comprises a second arm having a second hand at the proximal end of the cartridge and a second tail at the distal end of the cartridge. The first hand and the second hand define a receiver that includes a shelf positionable proximate to a ledge of a plate of a prosthesis and a tab positionable within a notch of the plate of the prosthesis. The ledge transitions directly into the notch of the prosthesis such that a shared surface of the ledge and the notch of the prosthesis is at least partially defined by an edge with a straight portion. The receiver includes a straight portion that corresponds to the straight portion of the edge of the shared surface of the prosthesis. The first tail and the second tail are insertable into an opening of an instrument. The first tail and the second tail define a slot and a cavity dimensioned to mate with a coupler of a locking shaft of the instrument. At least one of the first arm and the second arm includes a release tab that is able to engage a locking pin of a release driver of the instrument. Engagement of the release tab with the locking pin causes the receiver to release the prosthesis.

An eleventh aspect of the present disclosure is directed to a cartridge for an intervertebral disc prosthesis system. The cartridge comprises a first arm having a first hand at a proximal end of the cartridge and a first tail at a distal end of the cartridge. The cartridge comprises a second arm having a second hand at the proximal end of the cartridge and a second tail at the distal end of the cartridge. The first hand and the second hand define a receiver with at least one engagement feature able to engage with a corresponding engagement feature of a prosthesis. The first tail and the second tail are insertable into an opening of an instrument. The first tail and the second tail define a slot and a cavity dimensioned to mate with a coupler of a locking shaft of the instrument. At least one of the first arm and the second arm includes a release tab that is able to engage a locking pin of a release driver of the instrument. Engagement of the release tab with the locking pin causes the receiver to release the prosthesis. The first arm and the second arm each include a superior surface that is substantially planar with at least a portion of a superior surface of an upper plate of the prosthesis when the cartridge engages with the prosthesis. The first arm and the second arm each include an inferior surface that is substantially planar with at least a portion of an inferior surface of a lower plate of the prosthesis when the cartridge engages with the prosthesis. The first arm includes a first finger with a first exterior sidewall that is substantially planar with at least a portion of a first side surface of the upper plate of the prosthesis and at least a portion of a first side surface of the lower plate of the prosthesis when the cartridge engages with the prosthesis. The second arm includes a second finger with a second exterior sidewall that is substantially planar with at least a portion of a second side surface of the upper plate of the prosthesis and at least a portion of a second side surface of the lower plate of the prosthesis when the cartridge engages with the prosthesis.

A twelfth aspect of the present disclosure is directed to an instrument for an intervertebral disc prosthesis system. The instrument comprises an outer housing with an opening at a proximal end of the outer housing. The outer housing is actuatable to set a position of depth stops on the outer housing for a predetermined depth of implantation. The opening is able to receive an assembly including a cartridge and a prosthesis. The instrument comprises a locking shaft with a coupler at a proximal end of the locking shaft. The coupler is able to rotate between a first orientation and a second orientation. The coupler is insertable into a cavity defined within the cartridge when the assembly is inserted into the opening of the outer housing and when the coupler is in the first orientation. The coupler is dimensioned to mate with the cavity defined within the cartridge after the assembly is inserted into the opening of the outer housing and when the coupler is in the second orientation. The instrument comprises a release driver including a locking pin with an expansion bit. The release driver is actuatable to cause the expansion bit to engage at least one release tab of the cartridge to release the prosthesis from the cartridge after the assembly is inserted into the opening of the outer housing.

The instrument of the twelfth aspect may include, optionally, the locking pin with the expansion bit is inserted with the assembly into the opening of the outer housing such that the assembly includes the cartridge, the prosthesis, and the locking pin with the expansion bit. The locking pin couples to an actuator portion of the release driver after insertion of the assembly into the opening of the outer housing.

The instrument of the twelfth aspect may include one or more of the previous embodiments and, optionally, that the outer housing includes threading that is complementary to corresponding threading in an actuator, and wherein rotation of the actuator causes the complementary threading to engage and extend or retract the outer housing relative to the actuator when setting the position of the depth stops on the outer housing.

The instrument of the twelfth aspect may include one or more of the previous embodiments and, optionally, that the outer housing includes a plurality of markings separated in predetermined increments, the actuator includes an indicator that aligns with the plurality of markings, and a full rotation of the actuator represents an adjustment of one full shift in the predetermined increments.

The instrument of the twelfth aspect may include one or more of the previous embodiments and, optionally, that the outer housing includes a mechanical detent that engages a notch in the locking shaft when the depth stops are at a predetermined location based on a predetermined increment of a plurality of predetermined increments, and the mechanical detent engages an exterior surface of the locking shaft when the depth stops are outside of the predetermined location.

The instrument of the twelfth aspect may include one or more of the previous embodiments and, optionally, that the locking shaft includes an actuator able to rotate the coupler between the first orientation and the second orientation. When in the first orientation, the coupler is able to pass through a slot defined within in the cartridge and be located within the cavity defined within the cartridge when the assembly is inserted into the opening of the outer housing.

The instrument of the twelfth aspect may include one or more of the previous embodiments and, optionally, that the coupler has a length that is greater than a width. The width of the coupler is dimensioned to pass through the slot of the cartridge when the coupler is in the first orientation. The length of the coupler is dimensioned to mate with the cavity of the cartridge and prevent removal of the assembly from the opening of the outer housing when the coupler is in the second orientation.

The instrument of the twelfth aspect may include one or more of the previous embodiments and, optionally, a window in at least one of an upper tab of the outer housing and a lower tab of the outer housing. The mating of the coupler with the cavity of the cartridge when the coupler is in the second orientation is visible through the window.

The instrument of the twelfth aspect may include one or more of the previous embodiments and, optionally, that the actuator includes a first surface that provides a first indication when the coupler is in the first orientation, and the actuator includes a second surface that provides a second indication when the coupler is in the second orientation.

The instrument of the twelfth aspect may include one or more of the previous embodiments and, optionally, that the first surface has different contouring from the second surface, the first surface transitions into a surface of a second actuator, and the second surface is offset from the surface of the second actuator.

The instrument of the twelfth aspect may include one or more of the previous embodiments and, optionally, that the locking pin includes the expansion bit with a first width and at a proximal end, a middle portion with a second width that is less than the first width, and a distal portion.

The instrument of the twelfth aspect may include one or more of the previous embodiments and, optionally, that the release driver includes an actuator able to translate the expansion bit between a first position and a second position, and the at least one release tab is on one or more of a first arm and a second arm of the cartridge. When in the first position, the expansion bit is located in an end cavity defined within the first arm and the second arm, and the middle portion is located proximate to the at least one release tab such that the first arm and the second arm are in a first retracted position to hold the prosthesis. When in the second position, the expansion bit makes contact with the at least one release tab such that the first arm and the second arm are in a second expanded position to release the prosthesis.

The instrument of the twelfth aspect may include one or more of the previous embodiments and, optionally, that the distal portion of the locking pin and an actuator portion of the release driver are couplable via an interlocking assembly when the assembly is inserted into the opening of the outer housing.

The instrument of the twelfth aspect may include one or more of the previous embodiments and, optionally, that the interlocking assembly includes complementary threading of the distal portion of the locking pin and the actuator portion of the release driver. Actuation of the actuator translates the locking pin by changing an amount of engaged complementary threading of the interlocking assembly.

The instrument of the twelfth aspect may include one or more of the previous embodiments and, optionally, that the locking shaft is lockable by a toggle positioned within a slot in the outer housing via a force applied by at least one biasing element on the toggle.

The instrument of the twelfth aspect may include one or more of the previous embodiments and, optionally, that prior to insertion of the assembly into the opening of the outer housing, the coupler is dimensioned to mate within a cavity defined within a body of a trial implant when the coupler is in the second orientation and after the trial implant is inserted into the opening of the outer housing. The trial implant includes a head dimensioned to correspond to at least one of a predetermined depth, width, or height of the prosthesis of the assembly.

The instrument of the twelfth aspect may include one or more of the previous embodiments and, optionally, a window in at least one of an upper tab of the outer housing and a lower tab of the outer housing. The mating of the coupler with the cavity of the trial implant when the coupler is in the second orientation is visible through the window.

The instrument of the twelfth aspect may include one or more of the previous embodiments and, optionally, that the depth stops define a channel for an osteotome for use during trialing with the trial implant.

A thirteenth aspect of the present disclosure is directed to an instrument for an intervertebral disc prosthesis system. The instrument comprises an outer housing with an opening at a proximal end of the outer housing. The outer housing is actuatable to set a position of depth stops on the outer housing for a predetermined depth of implantation. The opening is able to receive an assembly including a cartridge, a prosthesis, and a locking pin including an expansion bit. The instrument comprises a locking shaft with a coupler at a proximal end of the locking shaft. The coupler is able to rotate between a first orientation and a second orientation. The coupler is insertable into a cavity defined within the cartridge when the assembly is inserted into the opening of the outer housing and when the coupler is in the first orientation. The coupler is dimensioned to mate with the cavity defined within the cartridge after the assembly is inserted into the opening of the outer housing and when the coupler is in the second orientation. The instrument comprises a release driver including an actuator portion that is couplable with a distal portion of the locking pin via an interlocking assembly when the assembly is inserted into the opening of the outer housing. The release driver is actuatable to cause the expansion bit to engage at least one release tab of the cartridge to release the prosthesis from the cartridge after the assembly is inserted into the opening of the outer housing.

A fourteenth aspect of the present disclosure is directed to an instrument for an intervertebral disc prosthesis system. The instrument comprises an outer housing with an opening at a proximal end of the outer housing. The outer housing is actuatable with a first actuator to set a position of depth stops on the outer housing for a predetermined depth of implantation. The opening is able to receive an assembly including a cartridge and a prosthesis. The instrument comprises a locking shaft with a coupler at a proximal end of the locking shaft. The coupler is able to rotate between a first orientation and a second orientation with a second actuator. The coupler is insertable into a cavity defined within the cartridge when the assembly is inserted into the opening of the outer housing and when the coupler is in the first orientation. The coupler is dimensioned to mate with the cavity defined within the cartridge after the assembly is inserted into the opening of the outer housing and when the coupler is in the second orientation. The instrument comprises a release driver including a locking pin with an expansion bit. The release driver is actuatable with a third actuator to cause the expansion bit to engage at least one release tab of the cartridge to release the prosthesis from the cartridge after the assembly is inserted into the opening of the outer housing.

A sixteenth aspect of the present disclosure is directed to an intervertebral disc prosthesis system. The system comprises an instrument including an outer housing with an opening at a proximal end of the outer housing, a locking shaft with a coupler at a proximal end of the locking shaft, and a release driver with an expansion bit at a proximal end of the release driver. The system comprises a cartridge with at least one release tab and a receiver defined at a proximal end of the cartridge. The system comprises a prosthesis having a distal end that is insertable into the receiver to form an assembly with the cartridge. A distal end of the cartridge is insertable into the opening at the proximal end of the instrument. The locking shaft is actuatable between a first orientation and a second orientation, where the coupler of the locking shaft is insertable into the distal end of the cartridge when the locking shaft is in the first orientation, and where the coupler of the locking shaft is retained within the distal end of the cartridge when the locking shaft is in the second orientation. Actuation of the release driver causes the expansion bit to engage the least one release tab to release the prosthesis from the receiver of the cartridge.

The system of the sixteenth aspect may include, optionally, an osteotome insertable into a channel on the outer housing; and a trial implant insertable into the opening at the proximal end of the outer housing when the cartridge is not inserted into the opening.

A seventeenth aspect of the present disclosure is to provide a method for implantation of an intervertebral disc prosthesis. The method may include, but is not limited to, coupling a prosthesis to a cartridge via at least one engagement feature to form an assembly. The method may include, but is not limited to, inserting the assembly into an opening in an outer housing of an instrument and engaging the cartridge with a coupler of a locking shaft of the instrument. The method may include, but is not limited to, releasing the prosthesis from the cartridge following implantation of the prosthesis by engaging at least one release tab of the cartridge with an expansion bit of a release driver of the instrument.

The method of the seventeenth aspect may include, optionally, prior to inserting the assembly into the opening in the outer housing, inserting a trial implant into the opening in the outer housing of the instrument. The method may include, optionally, prior to inserting the assembly into the opening in the outer housing, marking a midline for the implantation of the prosthesis with an osteotome set within a channel defined by at least one depth stop on the outer housing.

The method of the seventeenth aspect may include one or more of the previous embodiments and, optionally, prior to marking the midline, adjusting a depth of the at least one depth stop on the outer housing.

The method of the seventeenth aspect may include one or more of the previous embodiments and, optionally, that the assembly further includes a locking pin of the release driver. The locking pin is able to engage with an actuator portion of the release driver when the assembly is inserted into the opening of the outer housing. The expansion bit is a component of the locking pin.

The phrases “at least one,” “one or more,” and “and/or,” as used herein, are open-ended expressions that are both conjunctive and disjunctive in operation. For example, each of the expressions “at least one of A, B and C,” “at least one of A, B, or C,” “one or more of A, B, and C,” “one or more of A, B, or C” and “A, B, and/or C” means A alone, B alone, C alone, A and B together, A and C together, B and C together, or A, B and C together.

Unless otherwise indicated, all numbers expressing quantities, dimensions, conditions, and so forth used in the specification and claims are to be understood as being modified in all instances by the term “about.”

The use of “substantially” in the present disclosure, when referring to a measurable quantity (e.g., a diameter or other distance) and used for purposes of comparison, is intended to mean within (e.g., plus or minus) 5% of the comparative quantity. The terms “substantially similar to,” “substantially the same as,” and “substantially equal to,” as used herein, should be interpreted as if explicitly reciting and encompassing the special case in which the items of comparison are “similar to,”“the same as”and “equal to,”respectively.

As used herein, unless otherwise specified, the terms “about,” “approximately,” etc., when used in relation to numerical limitations or ranges, mean that the recited limitation or range may vary by up to (e.g., plus or minus) 15%, and in some instance by up to (e.g., plus or minus) 10%. By way of non-limiting example, “about 750” can mean as little as 675 or as much as 825, or any value therebetween. When used in relation to ratios or relationships between two or more numerical limitations or ranges, the terms “about,” “approximately,” etc. mean that each of the limitations or ranges may vary by up to (e.g., plus or minus) 10%. By way of non-limiting example, a statement that two quantities are “approximately equal” can mean that a ratio between the two quantities is as little as 0.9:1.1 or as much as 1.1:0.9 (or any value therebetween), and a statement that a four-way ratio is “about 5:3:1:1” can mean that the first number in the ratio can be any value of at least 4.5 and no more than 5.5, the second number in the ratio can be any value of at least 2.7 and no more than 3.3, and so on.

The term “a” or “an” entity, as used herein, refers to one or more of that entity. As such, the terms “a”(or “an”), “one or more”and “at least one”can be used interchangeably herein.

The use of “including,” “comprising,” or “having” and variations thereof herein is meant to encompass the items listed thereafter and equivalents thereof as well as additional items. Accordingly, the terms “including,” “comprising,” or “having” and variations thereof can be used interchangeably herein. The use of “engaged with” and variations thereof herein is meant to encompass any direct or indirect connections between components.

It shall be understood that the term “means” as used herein shall be given its broadest possible interpretation in accordance with 35 U.S.C. § 112(f). Accordingly, a claim incorporating the term “means” shall cover all structures, materials, or acts set forth herein, and all of the equivalents thereof. Further, the structures, materials, or acts and the equivalents thereof shall include all those described in the summary, brief description of the drawings, detailed description, abstract, and claims themselves.

All external references are hereby incorporated by reference in their entirety whether explicitly stated or not.

These and other advantages will be apparent from the disclosure contained herein. The above-described embodiments, objectives, and configurations are neither complete nor exhaustive. The Summary is neither intended nor should it be construed as being representative of the full extent and scope of the present disclosure. Moreover, references made herein to “the present disclosure,” or aspects thereof should be understood to mean certain embodiments of the present disclosure and should not necessarily be construed as limiting all embodiments to a particular description. The present disclosure is set forth in various levels of detail in the Summary as well as in the attached drawings and the Detailed Description and no limitation as to the scope of the present disclosure is intended by either the inclusion or non-inclusion of elements, components, etc. in this Summary. Additional aspects of the present disclosure will become more readily apparent from the Detailed Description, particularly when taken together with the drawings.

It is to be appreciated that any embodiment, feature, or aspect described herein can be claimed in combination with any other embodiment(s), feature(s), or aspect(s) as described herein, regardless of whether the features or aspects come from the same described embodiment. For example, any one or more aspects described herein can be combined with any other one or more aspects described herein. In addition, any one or more features described herein can be combined with any other one or more features described herein. Further, any one or more embodiments described herein can be combined with any other one or more embodiments described herein.

### BRIEF DESCRIPTION OF THE DRAWINGS

Those of skill in the art will recognize that the following description is merely illustrative of the principles of the disclosure, which may be applied in various ways to provide many different alternative embodiments. This description is made for illustrating the general principles of the teachings of this disclosure and is not meant to limit the inventive concepts disclosed herein.

The accompanying drawings, which are incorporated in and constitute a part of the specification, illustrate embodiments and together with the general description of the disclosure given above and the detailed description of the drawings given below, serve to explain the principles of the disclosure.

FIG. 1A illustrates a perspective view of an intervertebral disc prosthesis system, the system including a prosthesis, a cartridge, and an instrument, in accordance with one or more embodiments of the present disclosure;

FIG. 1B illustrates a left side elevation view of the system of FIG. 1A;

FIG. 1C illustrates a right side elevation view of the system of FIG. 1A;

FIG. 1D illustrates a top plan view of the system of FIG. 1A;

FIG. 1E illustrates a bottom plan view of the system of FIG. 1A;

FIG. 1F illustrates a front elevation view of the system of FIG. 1A;

FIG. 1G illustrates a rear elevation view of the system of FIG. 1A;

FIG. 2A illustrates a first perspective view of the prosthesis of the system of FIG. 1A, in accordance with one or more embodiments of the present disclosure;

FIG. 2B illustrates a second perspective view of the prosthesis of FIG. 2A;

FIG. 2C illustrates a first perspective view of the prosthesis of FIG. 2A in an exploded configuration;

FIG. 2D illustrates a second perspective view of the prosthesis of FIG. 2A in an exploded configuration;

FIG. 2E illustrates a top plan view of the prosthesis of FIG. 2A;

FIG. 2F illustrates a bottom plan view of the prosthesis of FIG. 2A;

FIG. 2G illustrates a front elevation view of the prosthesis of FIG. 2A;

FIG. 2H illustrates a rear elevation view of the prosthesis of FIG. 2A;

FIG. 2I illustrates a left side elevation view of the prosthesis of FIG. 2A;

FIG. 2J illustrates a right side elevation view of the prosthesis of FIG. 2A;

FIG. 2K illustrates a cross-section of a front elevation view of the prosthesis of FIG. 2G;

FIG. 2L illustrates a cross-section of the left side elevation view of the prosthesis of FIG. 2I;

FIG. 3A illustrates a perspective view of a cartridge of the system of FIG. 1A, in accordance with one or more embodiments of the present disclosure;

FIG. 3B illustrates a front elevation view of the cartridge of FIG. 3A;

FIG. 3C illustrates a rear elevation view of the cartridge of FIG. 3A;

FIG. 3D illustrates a top plan view of the cartridge of FIG. 3A;

FIG. 3E illustrates a bottom plan view of the cartridge of FIG. 3A;

FIG. 3F illustrates a left side elevation view of the cartridge of FIG. 3A;

FIG. 3G illustrates a right side elevation view of the cartridge of FIG. 3A;

FIG. 3H illustrates a cross-section of a perspective view of the cartridge of FIG. 3A;

FIG. 4A illustrates a perspective view of an instrument of the system of FIG. 1A, in accordance with one or more embodiments of the present disclosure;

FIG. 4B illustrates a top plan view of the instrument of FIG. 4A;

FIG. 4C illustrates a bottom plan view of the instrument of FIG. 4A;

FIG. 4D illustrates a cross-section of a side elevation view of the instrument of FIG. 4A;

FIG. 4E illustrates a perspective view of the instrument of FIG. 4A with an outer housing removed;

FIG. 4F illustrates a left side elevation view of the instrument of FIG. 4A;

FIG. 4G illustrates a right side elevation view of the instrument of FIG. 4A;

FIG. 4H illustrates a front elevation view of the instrument of FIG. 4A;

FIG. 4I illustrates a rear elevation view of the instrument of FIG. 4A;

FIG. 5A illustrates a perspective view of the cartridge of FIG. 3A proximate to the prosthesis of FIG. 2A, in accordance with one or more embodiments of the present disclosure;

FIG. 5B illustrates a perspective view of the cartridge engaging the prosthesis of FIG. 5A, to form a cartridge and prosthesis assembly;

FIG. 5C illustrates a top plan view of the cartridge and prosthesis assembly of FIG. 5B;

FIG. 5D illustrates a bottom plan view of the cartridge and prosthesis assembly of FIG. 5B;

FIG. 5E illustrates a perspective view of the instrument of FIG. 4A engaging the cartridge and prosthesis assembly of FIG. 5B;

FIG. 5F illustrates a top plan view of the prosthesis, cartridge, and a portion of the instrument of FIG. 5E;

FIG. 5G illustrates a perspective view of the prosthesis, cartridge, and a variation of the portion of the instrument of FIG. 5F;

FIG. 5H illustrates a perspective view of the prosthesis, cartridge, and a variation of the portion of the instrument of FIG. 5F;

FIG. 6A illustrates a perspective view of the instrument of the system of FIG. 1A with a trial implant and an osteotome, in accordance with one or more embodiments of the present disclosure;

FIG. 6B illustrates a left side elevation view of the system of FIG. 6A;

FIG. 6C illustrates a right side elevation view of the system of FIG. 6A;

FIG. 6D illustrates a top plan view of the system of FIG. 6A;

FIG. 6E illustrates a bottom plan view of the system of FIG. 6A;

FIG. 6F illustrates a front elevation view of the system of FIG. 6A;

FIG. 6G illustrates a rear elevation view of the system of FIG. 6A;

FIG. 7A illustrates a perspective view of the trial implant of the system of FIG. 6A, in accordance with one or more embodiments of the present disclosure;

FIG. 7B illustrates a left side elevation view of the trial implant of FIG. 7A;

FIG. 7C illustrates a right side elevation view of the trial implant of FIG. 7A;

FIG. 7D illustrates a top plan view of the trial implant of FIG. 7A;

FIG. 7E illustrates a bottom plan view of the trial implant of FIG. 7A;

FIG. 7F illustrates a front elevation view of the trial implant of FIG. 7A;

FIG. 7G illustrates a rear elevation view of the trial implant of FIG. 7A;

FIG. 8 illustrates a perspective view of the osteotome of the system of FIG. 6A, in accordance with one or more embodiments of the present disclosure;

FIG. 9A illustrates a flow diagram of a method or process for the operation and use of the system of FIGS. 6A and 17A, in accordance with one or more embodiments of the present disclosure;

FIG. 9B illustrates a flow diagram of a method or process for the operation and use of the system of FIGS. 1A and 12A, in accordance with one or more embodiments of the present disclosure;

FIG. 10A illustrates a first perspective view of a variation of a prosthesis for use with the instrument of FIG. 1A, in accordance with one or more embodiments of the present disclosure;

FIG. 10B illustrates a second perspective view of the prosthesis of FIG. 10A;

FIG. 10C illustrates a first perspective view of the prosthesis of FIG. 10A in an exploded configuration;

FIG. 10D illustrates a second perspective view of the prosthesis of FIG. 10A in an exploded configuration;

FIG. 10E illustrates a top plan view of the prosthesis of FIG. 10A;

FIG. 10F illustrates a bottom plan view of the prosthesis of FIG. 10A;

FIG. 10G illustrates a front elevation view of the prosthesis of FIG. 10A;

FIG. 10H illustrates a rear elevation view of the prosthesis of FIG. 10A;

FIG. 10I illustrates a left side elevation view of the prosthesis of FIG. 10A;

FIG. 10J illustrates a right side elevation view of the prosthesis of FIG. 10A;

FIG. 11A illustrates a perspective view of a variation of a cartridge for use with the instrument of FIG. 1A, in accordance with one or more embodiments of the present disclosure;

FIG. 11B illustrates a top plan view of a cartridge and prosthesis assembly including the prosthesis of FIG. 10A and the cartridge of FIG. 11A, in accordance with one or more embodiments of the present disclosure;

FIG. 11C illustrates a bottom plan view of the cartridge and prosthesis assembly of FIG. 11B;

FIG. 12A illustrates a perspective view of an intervertebral disc prosthesis system, the system including a prosthesis, a cartridge, and an instrument, in accordance with one or more embodiments of the present disclosure;

FIG. 12B illustrates a left side elevation view of the system of FIG. 12A;

FIG. 12C illustrates a right side elevation view of the system of FIG. 12A;

FIG. 12D illustrates a top plan view of the system of FIG. 12A;

FIG. 12E illustrates a bottom plan view of the system of FIG. 12A;

FIG. 12F illustrates a front elevation view of the system of FIG. 12A;

FIG. 12G illustrates a rear elevation view of the system of FIG. 12A;

FIG. 13A illustrates a first perspective view of the prosthesis of the system of FIG. 12A, in accordance with one or more embodiments of the present disclosure;

FIG. 13B illustrates a second perspective view of the prosthesis of FIG. 13A;

FIG. 13C illustrates a first perspective view of the prosthesis of FIG. 13A in an exploded configuration;

FIG. 13D illustrates a second perspective view of the prosthesis of FIG. 13A in an exploded configuration;

FIG. 13E illustrates a top plan view of the prosthesis of FIG. 13A;

FIG. 13F illustrates a bottom plan view of the prosthesis of FIG. 13A;

FIG. 13G illustrates a front elevation view of the prosthesis of FIG. 13A;

FIG. 13H illustrates a rear elevation view of the prosthesis of FIG. 13A;

FIG. 13I illustrates a left side elevation view of the prosthesis of FIG. 13A;

FIG. 13J illustrates a right side elevation view of the prosthesis of FIG. 13A;

FIG. 13K illustrates a cross-section of a front elevation view of the prosthesis of FIG. 13G;

FIG. 13L illustrates a cross-section of the left side elevation view of the prosthesis of FIG. 13I;

FIG. 14A illustrates a perspective view of a cartridge of the system of FIG. 12A, in accordance with one or more embodiments of the present disclosure;

FIG. 14B illustrates a front elevation view of the cartridge of FIG. 14A;

FIG. 14C illustrates a rear elevation view of the cartridge of FIG. 14A;

FIG. 14D illustrates a top plan view of the cartridge of FIG. 14A;

FIG. 14E illustrates a bottom plan view of the cartridge of FIG. 14A;

FIG. 14F illustrates a left side elevation view of the cartridge of FIG. 14A;

FIG. 14G illustrates a right side elevation view of the cartridge of FIG. 14A;

FIG. 15A illustrates a perspective view of an instrument of the system of FIG. 12A, in accordance with one or more embodiments of the present disclosure;

FIG. 15B illustrates a top plan view of the instrument of FIG. 15A;

FIG. 15C illustrates a bottom plan view of the instrument of FIG. 15A;

FIG. 15D illustrates a cross-section of a side elevation view of the instrument of FIG. 15A;

FIG. 15E illustrates a perspective view of the instrument of FIG. 15A with an outer housing removed;

FIG. 15F illustrates a left side elevation view of the instrument of FIG. 15A;

FIG. 15G illustrates a right side elevation view of the instrument of FIG. 15A;

FIG. 15H illustrates a front elevation view of the instrument of FIG. 15A;

FIG. 15I illustrates a rear elevation view of the instrument of FIG. 15A;

FIG. 16A illustrates a perspective view of the cartridge of FIG. 14A proximate to the prosthesis of FIG. 13A, in accordance with one or more embodiments of the present disclosure;

FIG. 16B illustrates a perspective view of the cartridge of FIG. 14A engaging the prosthesis of FIG. 13A, to form a cartridge and prosthesis assembly;

FIG. 16C illustrates a top plan view of the cartridge and prosthesis assembly of FIG. 16B;

FIG. 16D illustrates a bottom plan view of the cartridge and prosthesis assembly of FIG. 16B;

FIG. 16E illustrates a perspective view of the instrument of FIG. 15A engaging the cartridge and prosthesis assembly of FIG. 16B;

FIG. 16F illustrates a side elevation view of the cartridge and prosthesis assembly of FIG. 16B;

FIG. 16G illustrates a side elevation view of a variation of the cartridge and prosthesis assembly of FIG. 16B, including the cartridge of FIG. 12A and a variation of the prosthesis of FIG. 12A;

FIG. 17A illustrates a perspective view of the instrument of the system of FIG. 12A with a trial implant, in accordance with one or more embodiments of the present disclosure;

FIG. 17B illustrates a left side elevation view of the system of FIG. 17A;

FIG. 17C illustrates a right side elevation view of the system of FIG. 17A;

FIG. 17D illustrates a top plan view of the system of FIG. 17A;

FIG. 17E illustrates a bottom plan view of the system of FIG. 17A;

FIG. 17F illustrates a front elevation view of the system of FIG. 17A;

FIG. 17G illustrates a rear elevation view of the system of FIG. 17A;

FIG. 18A illustrates a perspective view of the trial implant of the system of FIG. 17A, in accordance with one or more embodiments of the present disclosure;

FIG. 18B illustrates a left side elevation view of the trial implant of FIG. 18A;

FIG. 18C illustrates a right side elevation view of the trial implant of FIG. 18A;

FIG. 18D illustrates a top plan view of the trial implant of FIG. 18A;

FIG. 18E illustrates a bottom plan view of the trial implant of FIG. 18A;

FIG. 18F illustrates a front elevation view of the trial implant of FIG. 18A;

FIG. 18G illustrates a rear elevation view of the trial implant of FIG. 18A;

FIG. 19A illustrates a first perspective view of a variation of a prosthesis for use with the instrument of FIG. 12A, in accordance with one or more embodiments of the present disclosure;

FIG. 19B illustrates a second perspective view of the prosthesis of FIG. 19A;

FIG. 19C illustrates a first perspective view of the prosthesis of FIG. 19A in an exploded configuration;

FIG. 19D illustrates a second perspective view of the prosthesis of FIG. 19A in an exploded configuration;

FIG. 19E illustrates a first left elevation view of the prosthesis of FIG. 19A;

FIG. 19F illustrates a second left elevation view of the prosthesis of FIG. 19A;

FIG. 19G illustrates a rear elevation view of the prosthesis of FIG. 19A;

FIG. 19H illustrates a front elevation view of the prosthesis of FIG. 19A;

FIG. 20A illustrates a first perspective view of a variation of a prosthesis for use with the instrument of FIG. 12A, in accordance with one or more embodiments of the present disclosure;

FIG. 20B illustrates a second perspective view of a variation of the prosthesis of FIG. 20A;

FIG. 20C illustrates a first perspective view of the prosthesis of FIG. 20A in an exploded configuration;

FIG. 20D illustrates a second perspective view of the prosthesis of FIG. 20A in an exploded configuration;

FIG. 20E illustrates a first left elevation view of the prosthesis of FIG. 20A;

FIG. 20F illustrates a second left elevation view of the prosthesis of FIG. 20A;

FIG. 20G illustrates a rear elevation view of the prosthesis of FIG. 20A; and

FIG. 20H illustrates a front elevation view of the prosthesis of FIG. 20A.

It should be understood that the drawings are not necessarily to scale, and various dimensions may be altered. In certain instances, details that are not necessary for an understanding of the disclosure or that render other details difficult to perceive may have been omitted. It should be understood, of course, that the disclosure is not necessarily limited to the particular embodiments illustrated herein.

### REFERENCE NUMBER COMPONENT

100 Intervertebral Disc Prosthesis System
102 Prosthesis
104 Cartridge
106 Instrument
108 Proximal End of Prosthesis
110 Distal End of Prosthesis
112 Proximal End of Cartridge
114 Distal End of Cartridge
116 Proximal End of Instrument
118 Distal End of Instrument
120 Depth Stop
122 Channel
124 Upper Tab of Instrument
126 Upper Wedge of Cartridge
128 Upper Surface of Cartridge
130 Opening of Instrument
132 Lower Tab of Instrument
134 Lower Wedge of Cartridge
136 Lower Surface of Cartridge
200 Upper Plate
202 Lower Plate
204 Insert
206 Superior Surface of Upper Plate
208 Inferior Surface of Upper Plate
210 Superior Surface of Lower Plate
212 Inferior Surface of Lower Plate
214 Superior Surface of Insert
216 Inferior Surface of Insert
217 Dome Region of Upper Plate
218 Convex Portion of Dome Region
220 Flat Portion Surrounding Convex Portion
222 Concave Portion of Dome Region
224 Flat Portion Surrounding Concave Portion
226 Rim of Inferior Surface of Upper Plate
228 Front Edge Chamfer of Upper Plate
230 Front Edge of Upper Plate
232 Side Edge Surface of Upper Plate
234 Side Edge of Upper Plate
236 Cartridge Engagement Feature of Upper Plate
238 Rear Edge of Upper Plate
240 Ledge of Cartridge Engagement Feature
242 Notch of Cartridge Engagement Feature
243 Portion of Superior Surface of Upper Plate
244 Set of Teeth on Upper Plate
246 Set of Alignment Teeth on Upper Plate
248 Protrusion on Lower Plate
250 Front Edge Chamfer of Lower Plate
252 Front Edge of Lower Plate
254 Side Edge of Lower Plate
256 Cartridge Engagement Feature of Lower Plate
258 Rear Edge of Lower Plate
260 Ledge of Cartridge Engagement Feature
262 Notch of Cartridge Engagement Feature
263 Portion of Inferior Surface of Lower Plate
264 Set of Teeth on Lower Plate
266 Set of Alignment Teeth on Lower Plate
268 Convex Region of Insert
270 Insert Recess
272 Insert Groove
274 Insert Endwall
300 First Arm of Cartridge
302 Second Arm of Cartridge
304 Proximal Section of First Arm
306 Middle Section of First Arm
308 Distal Section of First Arm
310 Proximal Section of Second Arm
312 Middle Section of Second Arm
314 Distal Section of Second Arm
316 Receiver
318 Hand of First Arm
320 Hand of Second Arm
322 Finger of First Hand
324 Finger of Second Hand
326 Upper Shelf of First Arm Hand
328 Upper Tab of First Arm Hand
330 Lower Shelf of First Arm Hand
332 Lower Tab of First Arm Hand
334 Upper Shelf of Second Arm Hand
336 Upper Tab of Second Arm Hand
338 Lower Shelf of Second Arm Hand
340 Lower Tab of Second Arm Hand
342 Proximal Crossmember of First Arm
344 Proximal Crossmember of Second Arm
346 Upper Surface of First Arm
348 Upper Surface of Second Arm
350 Lower Surface of First Arm
352 Lower Surface of Second Arm
354 Middle Crossmember of First Arm
356 Middle Crossmember of Second Arm
358 Cavity of Middle Section
360 Release Tab of First Arm
362 Release Tab of Second Arm
364 Distal Crossmember of First Arm
366 Distal Crossmember of Second Arm
368 Tail of First Arm
370 Tail of Second Arm
372 Slot of Distal End of Cartridge
374 Cavity of Distal End of Cartridge
376 Opening in Distal End of Cartridge
378 Support Member of First Arm
380 Support Member of Second Arm
400 Outer Housing
402 Locking Shaft
404 Release Driver
406 Body of Outer Housing
408 Proximal Section of Body
410 Middle Section of Body
412 Distal Section of Body
414 Actuator for Outer Housing
416 Actuator for Locking Shaft
418 Actuator for Release Driver
420 Alignment Feature
422 First Window of Proximal Section of Body
424 Second Window of Proximal Section of Body
426 Coupler of Locking Shaft
428 Middle Section of Coupler
430 Lobe of Coupler
432 Slot of Locking Shaft
434 Upper Surface of Proximal Section of Body
436 Lower Surface of Proximal Section of Body
438 Expansion Bit (or Proximal Portion of Locking Pin)
439 Middle Portion of Locking Pin
440 Interlocking Assembly for Locking Pin
441 Distal Portion of Locking Pin
442 Actuator Portion of Release Driver
444 Locking Pin of Release Driver
446 First Portion of Middle Section
448 Second Portion of Middle Section
450 Lock Assembly
452 Toggle of Lock Assembly
454 Slot in Outer Housing
456 Pin in Outer Housing
458 Aperture in Toggle
460 Protrusion of Toggle
462 Recess on Release Driver
464 Biasing Element
466 Marker on Outer Housing
468 Indicator on Actuator
470 Threading for Actuator
472 Aperture for Release Driver
474 Distal End Surface of Actuator
475 Proximal Member
477 Distal Shaft
500 First Edge of Receiver
501 Portion of First Edge of Receiver
502 Second Edge of Receiver
503 Portion of Second Edge of Receiver
504 Side Surface of First Arm
506 First Side Surface of Upper Plate
508 First Side Surface of Lower Plate
510 Side Surface of Second Arm
512 Second Side Surface of Upper Plate
514 Second Side Surface of Lower Plate
516 End Cavity in Cartridge
518 Proximal End of Release Driver
520 Proximal Portion of First Arm
522 Proximal Portion of Second Arm
524 Engagement Feature of Release Driver
526 Locking Tab of First Arm
528 First Locking Recess of Locking Pin
530 Locking Tab of Second Arm
532 Second Locking Recess of Locking Pin
534 Cartridge and Prosthesis Assembly
600 Trial Implant
602 Osteotome
604 Proximal End of Trial Implant
606 Distal End of Trial Implant
608 Proximal End of Osteotome
610 Distal End of Osteotome
612 Upper Wedge of Trial Implant
614 Upper Surface of Trial Implant
616 Lower Wedge of Trial Implant
618 Lower Surface of Trial Implant
620 Width of Trial Implant
700 Head of Trial Implant
702 Body of Trial Implant
704 Superior Surface of Head
706 Inferior Surface of Head
708 Sidewall of Head
710 Sidewall of Body
712 Transition Region of Trial Implant
714 Convex Portion of Dome Region
716 Flat Portion Surrounding Convex Portion
718 Front Edge Chamfer of Superior Surface of Head
720 Front Edge of Superior Surface of Head
722 Alignment Feature of Head
724 Front Edge Chamfer of Superior Surface of Head
726 Front Edge of Superior Surface of Head
728 Crossmember of Body of Trial Implant
730 First Tail of Body of Trial Implant
732 Second Tail of Body of Trial Implant
734 Slot of Distal End of Trial Implant
736 Cavity of Distal End of Trial Implant
738 Cavity in Body of Trial Implant
740 Trial Implant Markings
800 Body of Osteotome
802 Tip of Osteotome
900 Method or Process
902 Prepare a Patient for Trialing and Implantation
904 Trial the Patient with a Trial Implant and an Instrument to Define a Depth, Width, and/or Height of a Prosthesis
906 Mark a Midline on a Vertebral Body of the Patient Following Trialing with an Osteotome
908 Acquire a Prosthesis and a Cartridge
910 Method or Process
912 Perform Operations of Method or Process
914 Insert a Prosthesis and Cartridge Assembly into the Instrument
916 Implant the Prosthesis into the Patient
918 Engage the Depth Stop within the Patient During Implantation
920 Release the Prosthesis from the Cartridge and the Instrument
1002 Prosthesis
1004 Cartridge
1026 Prosthesis and Cartridge Assembly
1036 Cartridge Engagement Feature of Upper Plate
1038 Rear Edge of Upper Plate
1040 Ledge of Cartridge Engagement Feature
1042 Notch of Cartridge Engagement Feature
1043 Portion of Superior Surface of Upper Plate
1045 Rear Edge of Ledge
1047 Rear Surface of Notch
1056 Cartridge Engagement Feature of Lower Plate
1058 Rear Edge of Lower Plate
1060 Ledge of Cartridge Engagement Feature
1062 Notch of Cartridge Engagement Feature
1063 Portion of Inferior Surface of Lower Plate
1065 Rear Edge of Ledge
1067 Rear Surface of Notch
1100 First Edge of Receiver
1101 Portion of First Edge of Receiver
1102 Second Edge of Receiver
1103 Portion of Second Edge of Receiver
1126 Upper Shelf of First Arm Hand
1128 Upper Tab of First Arm Hand
1130 Lower Shelf of First Arm Hand
1132 Lower Tab of First Arm Hand
1134 Upper Shelf of Second Arm Hand
1136 Upper Tab of Second Arm Hand
1138 Lower Shelf of Second Arm Hand
1140 Lower Tab of Second Arm Hand
1200 Intervertebral Disc Prosthesis System
1202 Prosthesis
1204 Cartridge
1206 Instrument
1208 Proximal End of Prosthesis
1210 Distal End of Prosthesis
1212 Proximal End of Cartridge
1214 Distal End of Cartridge
1216 Proximal End of Instrument
1218 Distal End of Instrument
1220 Depth Stop
1222 Channel
1224 Upper Tab of Instrument
1226 Upper Wedge of Cartridge
1228 Upper Surface of Cartridge
1230 Opening of Instrument
1232 Lower Tab of Instrument
1234 Lower Wedge of Cartridge
1236 Lower Surface of Cartridge
1300 Upper Plate
1302 Lower Plate
1304 Insert
1306 Superior Surface of Upper Plate
1308 Inferior Surface of Upper Plate
1310 Superior Surface of Lower Plate
1312 Inferior Surface of Lower Plate
1314 Superior Surface of Insert
1316 Inferior Surface of Insert
1317 Dome Region of Upper Plate
1318 Convex Portion of Dome Region
1320 Flat Portion Surrounding Convex Portion
1322 Concave Portion of Dome Region
1324 Flat Portion Surrounding Concave Portion
1326 Rim of Inferior Surface of Upper Plate
1328 Front Edge Chamfer of Upper Plate
1330 Front Edge of Upper Plate
1332 Side Edge Surface of Upper Plate
1334 Side Edge of Upper Plate
1336 Cartridge Engagement Feature of Upper Plate
1338 Rear Edge of Upper Plate
1340 Ledge of Cartridge Engagement Feature
1342 Notch of Cartridge Engagement Feature
1343 Portion of Superior Surface of Upper Plate
1344 Set of Teeth on Upper Plate
1345 Rear Edge of Ledge
1347 Rear Surface of Notch
1346 Set of Alignment Teeth on Upper Plate
1348 Protrusion on Lower Plate
1350 Front Edge Chamfer of Lower Plate
1352 Front Edge of Lower Plate
1354 Side Edge of Lower Plate
1356 Cartridge Engagement Feature of Lower Plate
1358 Rear Edge of Lower Plate
1360 Ledge of Cartridge Engagement Feature
1362 Notch of Cartridge Engagement Feature
1363 Portion of Inferior Surface of Lower Plate
1364 Set of Teeth on Lower Plate
1366 Set of Alignment Teeth on Lower Plate
1368 Convex Region of Insert
1370 Insert Recess
1371 Insert Sidewall
1372 Insert Groove
1374 Insert Endwall
1400 First Arm of Cartridge
1402 Second Arm of Cartridge
1404 Proximal Section of First Arm
1406 Middle Section of First Arm
1408 Distal Section of First Arm
1410 Proximal Section of Second Arm
1412 Middle Section of Second Arm
1414 Distal Section of Second Arm
1416 Receiver
1417 First Edge of Receiver
1418 Hand of First Arm
1419 Portion of First Edge of Receiver
1420 Hand of Second Arm
1421 Second Edge of Receiver
1422 Finger of First Hand
1423 Portion of Second Edge of Receiver
1424 Finger of Second Hand
1426 Upper Shelf of First Arm Hand
1428 Upper Tab of First Arm Hand
1430 Lower Shelf of First Arm Hand
1432 Lower Tab of First Arm Hand
1434 Upper Shelf of Second Arm Hand
1436 Upper Tab of Second Arm Hand
1438 Lower Shelf of Second Arm Hand
1440 Lower Tab of Second Arm Hand
1442 Proximal Crossmember of First Arm
1444 Proximal Crossmember of Second Arm
1446 Upper Surface of First Arm
1448 Upper Surface of Second Arm
1450 Lower Surface of First Arm
1452 Lower Surface of Second Arm
1454 Middle Crossmember of First Arm
1456 Middle Crossmember of Second Arm
1458 Cavity of Middle Section
1460 Release Tab of First Arm
1462 Release Tab of Second Arm
1464 Distal Crossmember of First Arm
1466 Distal Crossmember of Second Arm
1468 Tail of First Arm
1470 Tail of Second Arm
1472 Slot of Distal End of Cartridge
1474 Cavity of Distal End of Cartridge
1476 Opening in Distal End of Cartridge
1478 Support Member of First Arm
1480 Support Member of Second Arm
1500 Outer Housing
1502 Locking Shaft
1504 Release Driver
1506 Body of Outer Housing
1508 Proximal Section of Body
1510 Middle Section of Body
1512 Distal Section of Body
1514 Actuator for Outer Housing
1516 Actuator for Locking Shaft
1518 Actuator for Release Driver
1520 Alignment Feature
1522 First Window of Proximal Section of Body
1524 Second Window of Proximal Section of Body
1526 Coupler of Locking Shaft
1528 Middle Section of Coupler
1530 Lobe of Coupler
1532 Slot of Locking Shaft
1534 Upper Surface of Proximal Section of Body
1536 Lower Surface of Proximal Section of Body
1538 Expansion Bit (or Proximal Portion of Locking Pin)
1539 Middle Portion of Locking Pin
1540 Interlocking Assembly for Locking Pin
1541 Distal Portion of Locking Pin
1542 Actuator Portion of Release Driver
1544 Locking Pin of Release Driver
1546 First Portion of Middle Section
1548 Second Portion of Middle Section
1550 Lock Assembly
1552 Toggle of Lock Assembly
1554 Slot in Outer Housing
1556 Pin in Outer Housing
1558 Aperture in Toggle
1560 Protrusion of Toggle
1562 Recess on Release Driver
1564 Biasing Element
1566 Marker on Outer Housing
1567 Tactile Indicator
1568 Indicator on Actuator
1569 Notch for Tactile Indicator
1570 Threading for Actuator
1571 Recess for Tactile Indicator
1572 Aperture for Release Driver
1574 Distal End Surface of Actuator
1575 Proximal Member
1576 First Surface of Actuator
1577 Distal Shaft
1578 Second Surface of Actuator
1579 Surface of Actuator
1580 Edge of Actuator
1582 Edge of Actuator
1584 Cavity
1586 Exterior Surface
1588 Engagement Feature on Exterior Surface
1600 First Edge of Receiver
1601 Portion of First Edge of Receiver
1602 Second Edge of Receiver
1603 Portion of Second Edge of Receiver
1604 Side Surface of First Arm
1606 First Side Surface of Upper Plate
1608 First Side Surface of Lower Plate
1610 Side Surface of Second Arm
1612 Second Side Surface of Upper Plate
1614 Second Side Surface of Lower Plate
1616 End Cavity in Cartridge
1618 Proximal End of Release Driver
1620 Proximal Portion of First Arm
1622 Proximal Portion of Second Arm
1624 Engagement Feature of Release Driver
1626 Locking Tab of First Arm
1628 First Locking Recess of Locking Pin
1630 Locking Tab of Second Arm
1632 Second Locking Recess of Locking Pin
1634 Cartridge and Prosthesis Assembly
1636 Prosthesis
1638 Cartridge and Prosthesis Assembly
1700 Trial Implant
1704 Proximal End of Trial Implant
1706 Distal End of Trial Implant
1712 Upper Wedge of Trial Implant
1714 Upper Surface of Trial Implant
1716 Lower Wedge of Trial Implant
1718 Lower Surface of Trial Implant
1720 Width of Trial Implant
1800 Head of Trial Implant
1802 Body of Trial Implant
1804 Superior Surface of Head
1806 Inferior Surface of Head
1808 Sidewall of Head
1810 Sidewall of Body
1812 Transition Region of Trial Implant
1814 Convex Portion of Dome Region
1816 Flat Portion Surrounding Convex Portion
1818 Front Edge Chamfer of Superior Surface of Head
1820 Front Edge of Superior Surface of Head
1822 Alignment Feature of Head
1824 Front Edge Chamfer of Superior Surface of Head
1826 Front Edge of Superior Surface of Head
1828 Crossmember of Body of Trial Implant
1830 First Tail of Body of Trial Implant
1832 Second Tail of Body of Trial Implant
1834 Slot of Distal End of Trial Implant
1836 Cavity of Distal End of Trial Implant
1838 Cavity in Body of Trial Implant
1902 Prosthesis
1908 Inferior Surface of Upper Plate
1917 Dome Region of Upper Plate
1922 Concave Portion of Dome Region
1924 Flat Portion Surrounding Concave Portion
1926 Rim of Inferior Surface of Upper Plate
1968 Convex Region of Insert
1969 Ring or Flange Portion of Insert
1975 Ring Angle
2002 Prosthesis
2008 Inferior Surface of Upper Plate
2017 Dome Region of Upper Plate
2022 Concave Portion of Dome Region
2024 Flat Portion Surrounding Concave Portion
2026 Rim of Inferior Surface of Upper Plate
2048 Protrusion
2068 Convex Region of Insert
2069 Ring or Flange Portion of Insert
2070 Insert Recess
2075 Ring Angle

### DETAILED DESCRIPTION

Although the following text sets forth a detailed description of numerous different embodiments, it should be understood that the legal scope of the description is defined by the words of the claims set forth at the end of this disclosure. The Detailed Description is to be construed as exemplary only and does not describe every possible embodiment since describing every possible embodiment of the intervertebral disc prosthesis systems and methods, would be impractical, if not impossible. Numerous alternative embodiments could be implemented, using either current technology or technology developed after the filing date of this patent, which would still fall within the scope of the claims. Additionally, any combination of features shown in the various figures can be used to create additional embodiments of the present disclosure. Thus, dimensions, aspects, and features of one embodiment of the intervertebral disc prosthesis systems and methods, can be combined with dimensions, aspects, and features of another embodiment of the intervertebral disc prosthesis systems and methods, to create the claimed embodiment.

In general, embodiments of the present disclosure are directed to an intervertebral disc prosthesis, and systems and methods for implantation of the same. The prosthesis is insertable between adjacent vertebral bodies, and includes an upper plate, a lower plate, and an insert enclosed therebetween. The insert is able to move within a cavity or space defined between the upper and lower plates to allow for repositioning during movement of the patient in which the prosthesis is implanted, but is also secured by features of the upper and/or lower plates to prevent slippage or loss of the insert from between the upper and lower plates when implanted.

Embodiments of the present disclosure are also directed to an instrument able to interface with a cartridge, where the cartridge is able to engage the prosthesis. Following engagement of the prosthesis by the cartridge to form a cartridge and prosthesis assembly, the instrument is able to interface with the cartridge via insertion of the cartridge into an outer housing of the instrument and actuation of a locking shaft of the instrument. The instrument can then be used to position the prosthesis within the implantation site. Once the prosthesis is positioned with a cavity defined between adjacent vertebral bodies, actuation of a release driver within the instrument causes the cartridge to disengage from the prosthesis, allowing for the removal of the cartridge and the instrument from the implantation site.

Embodiments of the present disclosure are directed to a portion of the instrument (e.g., a coupler of a locking shaft) to engage with the cartridge when the cartridge and prosthesis assembly is inserted into the instrument. With the insertion of the cartridge of the cartridge and prosthesis assembly within the instrument, the portion of the instrument engages the cartridge and prevents removal of the cartridge from the instrument.

Embodiments of the present disclosure are directed to providing at least a portion (e.g., a locking pin with an expansion bit of a release driver) of the instrument with the cartridge and prosthesis assembly. With the insertion of the cartridge of the cartridge and prosthesis assembly within the instrument, the portion of the instrument interfaces with a corresponding remaining portion of the instrument.

Embodiments of the present disclosure are also directed to trial implants and an osteotome for use during trialing and prior to implantation of the prosthesis. The trial implant includes a body that is dimensioned similar to the cartridge to interface with the instrument. The trial implant includes a head that is dimensioned similar to a particular prosthesis. Insertion of the trial implant with the instrument into the cavity defined between adjacent vertebral bodies allows a user to determine which particular prosthesis should be implanted within the cavity.

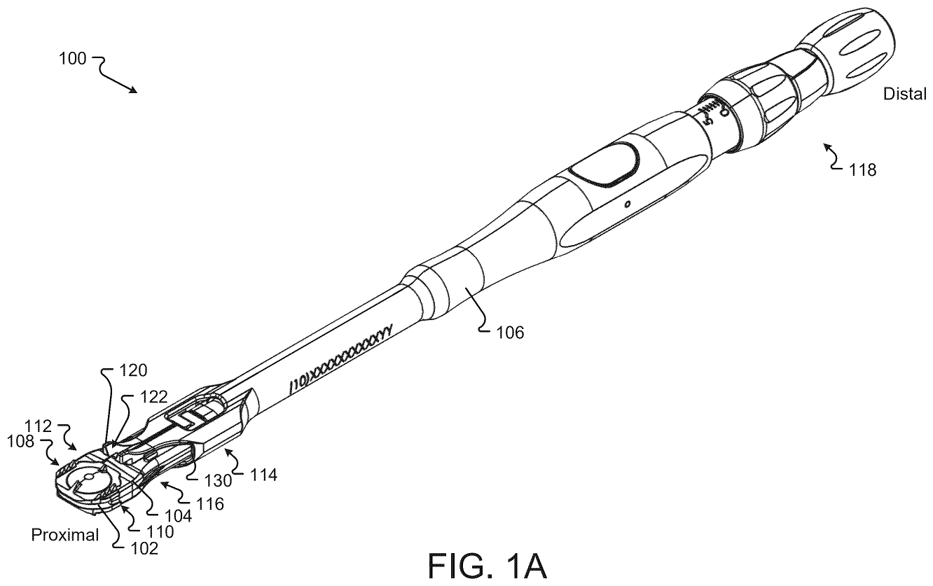

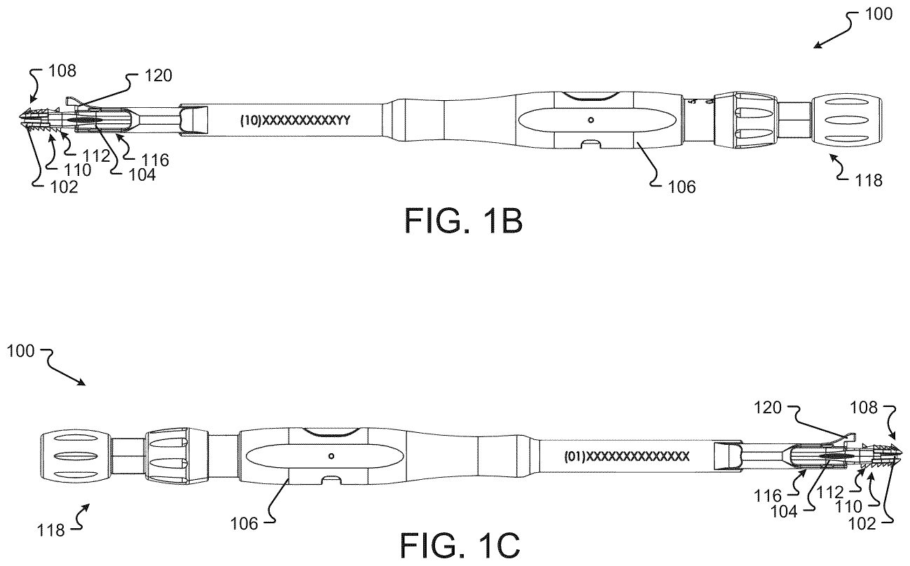

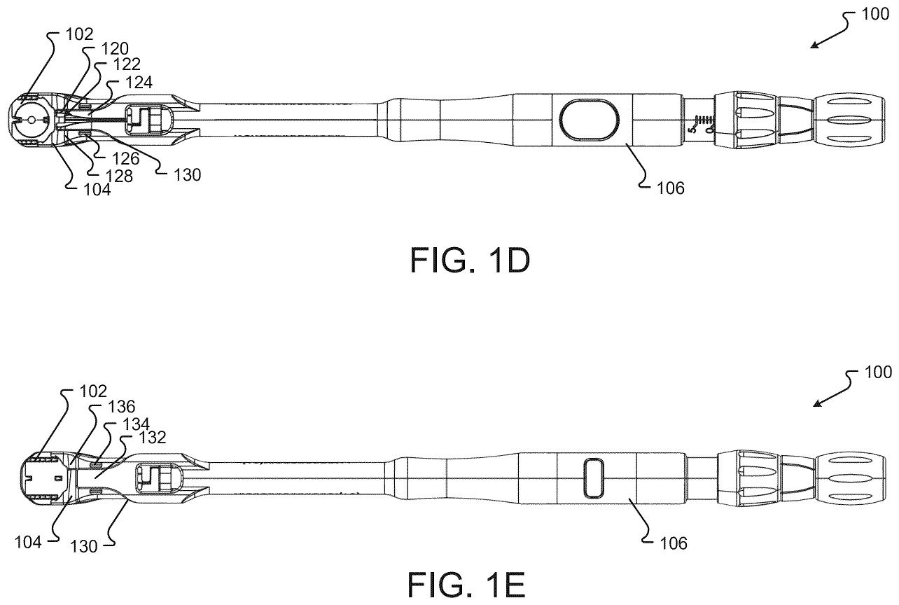

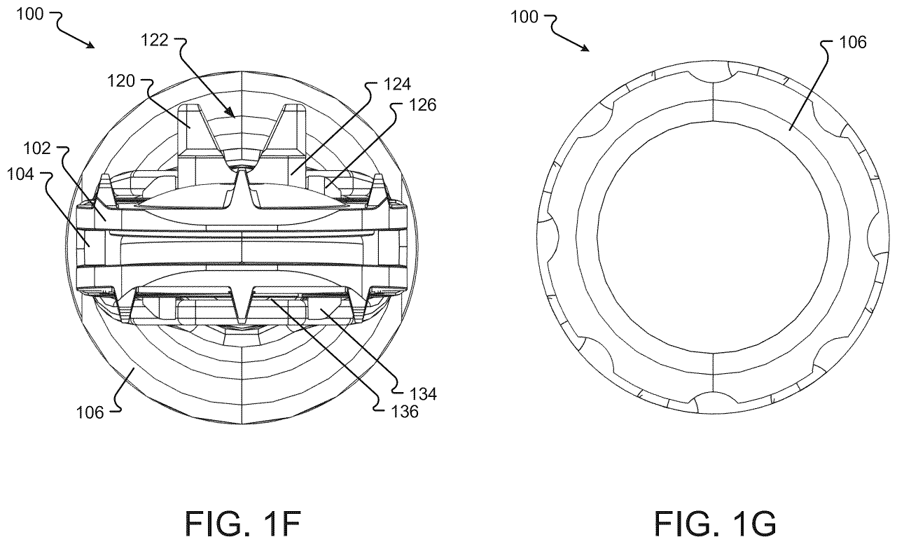

FIGS. 1A-1G in general illustrate an intervertebral disc prosthesis system 100 (or the system 100), in accordance with one or more embodiments of the present disclosure. The system 100 includes one or more of a prosthesis 102, a cartridge 104, and an instrument 106. Reference will be made to each of the subassemblies 102, 104, 106 in detail further herein. In addition, any and/or all of the subassemblies 102, 104, 106 may include further subassemblies as described in detail further herein, which are each considered subassemblies of the system 100.

As illustrated in FIGS. 1A-1G, the prosthesis 102 has a proximal end 108 and a distal end 110. The cartridge 104 has a proximal end 112 and a distal end 114. The instrument 106 has a proximal end 116 and a distal end 118.

The proximal end 112 of the cartridge 104 receives the distal end 110 of the prosthesis 102. The distal end 114 of the cartridge 104 is insertable into the proximal end 116 of the instrument 106. In some embodiments, the proximal end 112 of the cartridge 104 receives the distal end 110 of the prosthesis 102 prior to insertion of the distal end 114 of the cartridge 104 into the proximal end 116 of the instrument 106 (e.g., such that the cartridge 104 and the prosthesis 102 are inserted as an assembly into the instrument 106). In other embodiments, the proximal end 112 of the cartridge 104 receives the distal end 110 of the prosthesis 102 after insertion of the distal end 114 of the cartridge 104 into the proximal end 116 of the instrument 106.

In some embodiments, the proximal end 116 of the instrument 106 includes at least one depth stop 120 that defines a channel 122 on an upper tab 124. In some non-limiting examples, the channel 122 is defined by two depth stops 120.

The upper tab 124 of the instrument 106 is configured to engage at least one upper wedge 126 on an upper surface 128 of the cartridge 104, to assist in aligning the cartridge 104 within an opening 130 in the proximal end 116 of the instrument 106. For example, the upper surface 128 may include two upper wedges 126 that flank the upper tab 124 of the instrument 106 during insertion of the cartridge 104 into the opening 130 of the instrument 106.

In other embodiments, a lower tab 132 of the instrument 106 (e.g., at the proximal end 116 of the instrument 106) is configured to engage at least one lower wedge 134 on a lower surface 136 of the cartridge 104, to assist in aligning the cartridge 104 within the opening 130 in the proximal end 116 of the instrument 106. For example, the lower surface 136 may include two lower wedges 134 that flank the lower tab 132 of the instrument 106 during insertion of the cartridge 104 into the opening 130 of the instrument 106.

It is noted that the cartridge 104 may include the upper wedges 126 and/or the lower wedges 134 either individually or in combination, without departing from the scope of the present disclosure.

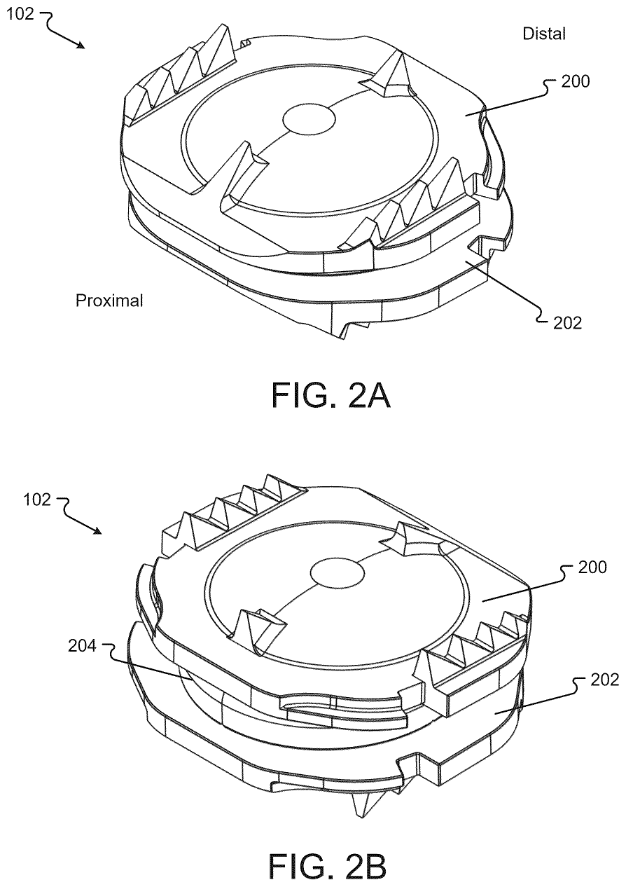

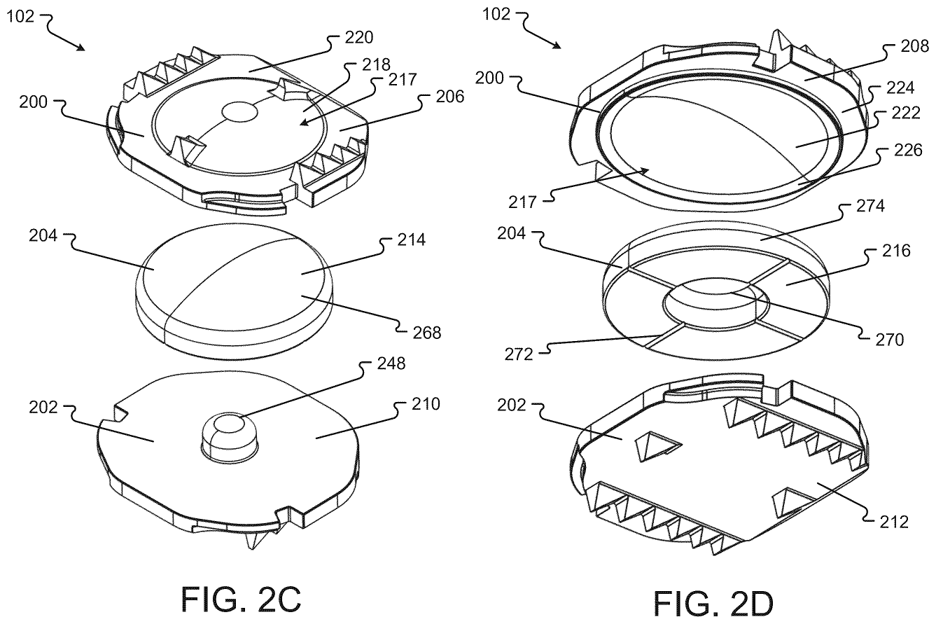

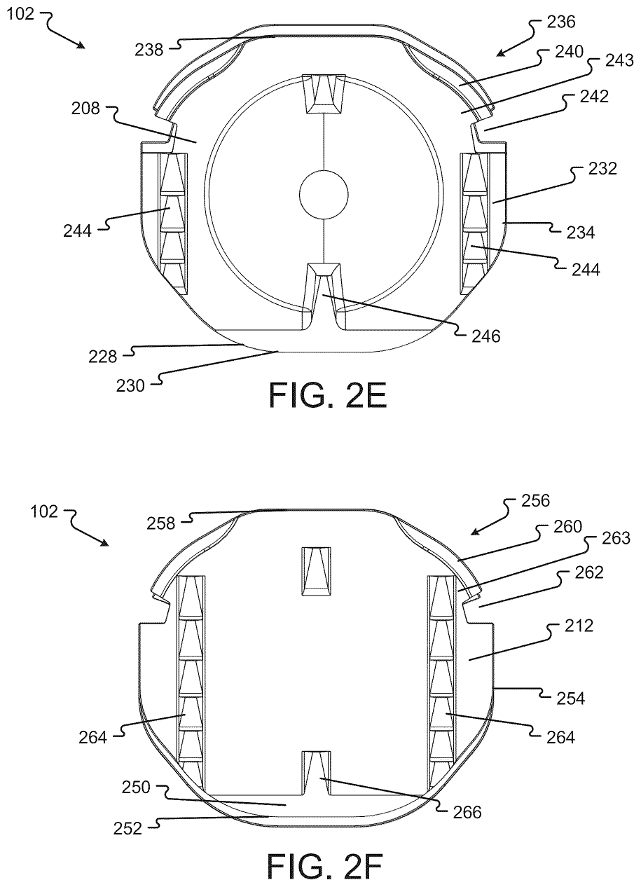

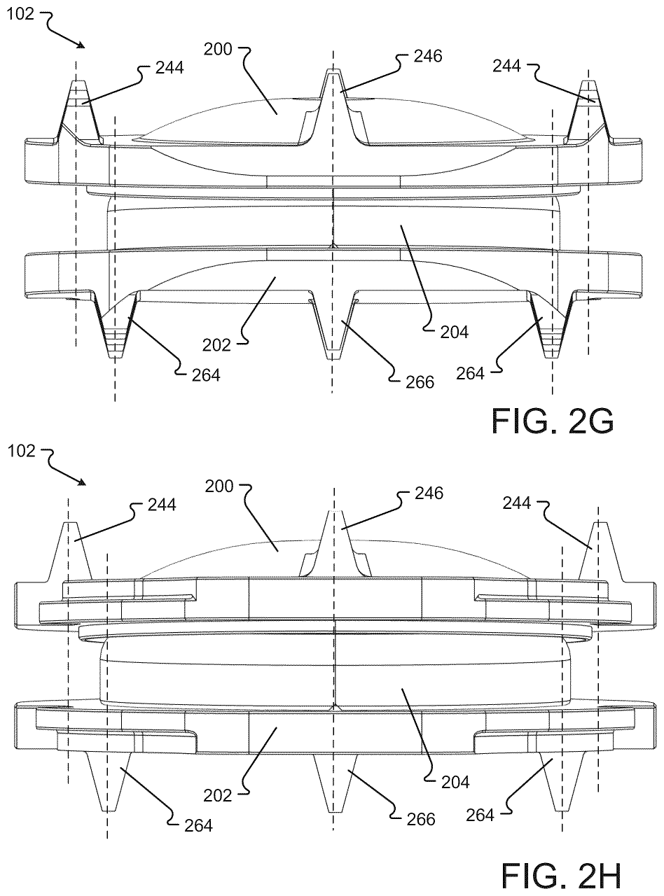

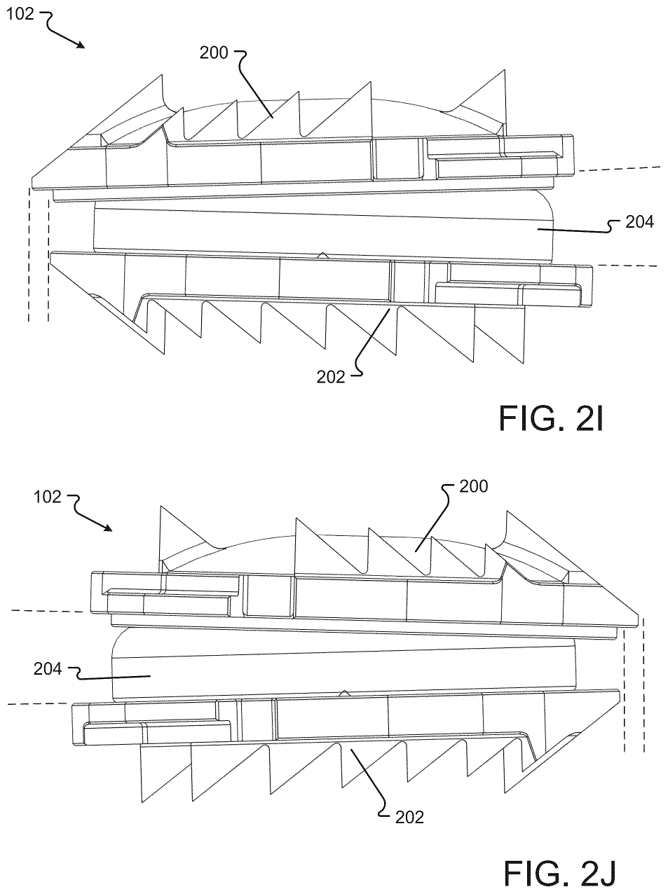

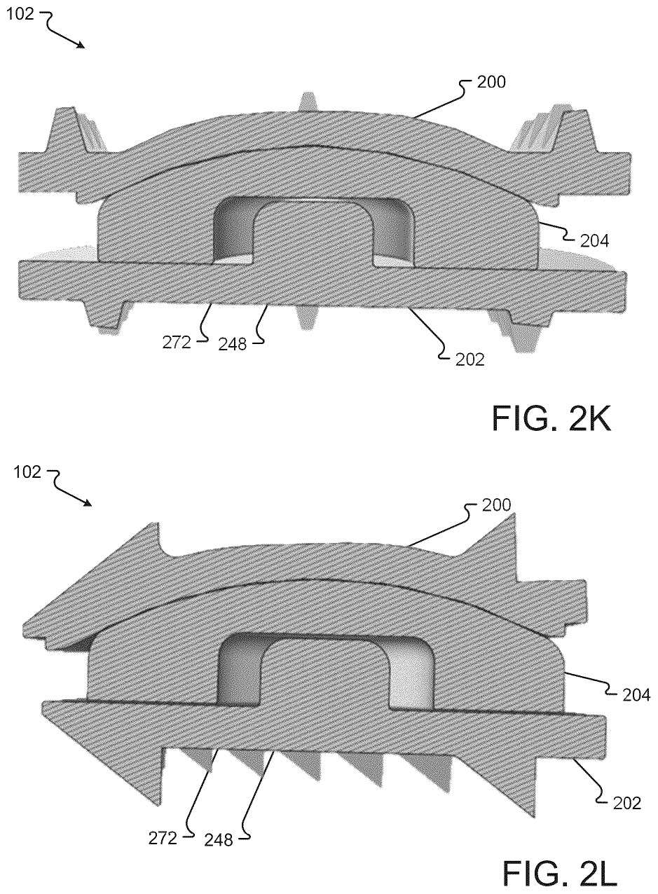

FIGS. 2A-2L in general illustrate the prosthesis 102 of the system 100, in accordance with one or more embodiments of the present disclosure. It should be understood that any embodiment of the prosthesis 102 illustrated in FIGS. 1A-1G may be directed to the prosthesis 102 illustrated in FIGS. 2A-2L, and vice versa, without departing from the scope of the present disclosure.

In embodiments, the prosthesis 102 includes an upper plate (or superior plate) 200, a lower plate (or inferior plate) 202, and an insert (or central core) 204. The upper plate 200 has a superior surface 206 and an inferior surface 208. The lower plate 202 has a superior surface 210 and an inferior surface 212. The insert 204 has a superior surface 214 and an inferior surface 216.

In some embodiments, at least one of the upper plate 200 and/or the lower plate 202 is fabricated from a metal or metal alloy. For example, the metal or metal alloy may include, but is not limited to, a Titanium (Ti) alloy. For instance, the metal or metal alloy may include an alloy comprising Titanium (Ti), Aluminum (Al), and Vanadium (V) (e.g., Ti₆Al₄V).

In some embodiments, at least one of the superior surface 206 of the upper plate 200 and/or the inferior surface 212 of the lower plate 202 is coated. For example, the coating may include, but is not limited to, a bioceramic. For instance, the bioceramic may include hydroxyapatite (Ca₁₀(PO₄)₆(OH)₂) (HAP), to create a Ti/HAP coating on the superior surface 206 of the upper plate 200 and/or the inferior surface 212 of the lower plate 202. In some instances, a bioceramic coating such as Ti/HAP may improve osseointegration between the prosthesis 102 and respective contact surfaces of the adjacent vertebral bodies between which the prosthesis 102 is implanted.

In some embodiments, the insert 204 is fabricated from a polymer. For example, the polymer may include, but is not limited to, a polyethylene (PE)-based polymer. For instance, the PE-based polymer may be infused with Vitamin E, to create a PE Vit E polymer.

In some embodiments, at least one of the inferior surface 208 of the upper plate 200 and/or the superior surface 210 of the lower plate 202 is coated. For example, the coating may include, but is not limited to, a friction enhancement coating. For instance, the friction enhancement coating reduces the friction coefficient and beneficially limits wear debris from forming within the implantation site. In addition, the friction enhancement coating reduces the possibility that the insert 204 exits the cavity defined between the upper plate 200 and the lower plate 202.

In embodiments, the upper plate 200 includes a dome region 217. In general, the dome region is contoured to conform to the superior surface 214 of the insert 204, which is optionally convex and dome-shaped. For instance, the superior surface 214 of the insert 204 may be contoured to conform to a substantially spherical portion for the convex dome shape.

For example, the dome region 217 may include a convex portion 218 at least partially surrounded by a substantially flat or planar portion 220 in the superior surface 206. The transition between the convex portion 218 and the substantially flat or planar portion 220 may be sloped and/or without a step-like change in height. Alternatively, the transition between the convex portion 218 and the substantially flat or planar portion 220 may have a step-like change in height.

By way of another example, the dome region 217 may include a concave portion 222 at least partially surrounded by a substantially flat or planar portion 224 in the inferior surface 208. The transition between the concave portion 222 and the substantially flat or planar portion 224 may have a step-like change in height. For example, the step-like change in height may be formed by a substantially flat or rounded rim 226 that extends away from (e.g., downward) from the inferior surface 208. Alternatively, the transition between the convex portion 218 and the substantially flat or planar portion 220 may be sloped and/or without a step-like change in height.

It should be understood that the upper plate 200 may include both portions 218, 222, or only one of the portions 218, 222, without departing from the scope of the present disclosure. For example, the upper plate 200 may include the concave portion 222 in the inferior surface 208, but a substantially flat or planar superior surface 206 without the convex portion 218.

In embodiments, the upper plate 200 includes a chamfer 228 on a front edge or leading edge 230. For example, the chamfer 228 may facilitate insertion of the prosthesis 102 into the cavity defined between the adjacent vertebral bodies during implantation. Optionally, the upper plate 200 includes a substantially flat or planar surface 232 on one or more side edges 234. For example, both side edges 234 may include the substantially flat or planar surface 232. It is noted that the substantially flat or planar surface 232 may facilitate insertion of the prosthesis 102 into the cavity defined between the adjacent vertebral bodies during implantation.

In embodiments, the upper plate 200 includes at least one cartridge engagement feature 236 on a rear edge or trailing edge 238. For example, the upper plate 200 includes two opposing cartridge engagement features 236 on the rear edge 238. The at least one cartridge engagement feature 236 includes a ledge 240 within the superior surface 206, and a notch 242 through the thickness of the upper plate 200 and between the superior surface 206 and the inferior surface 208. In this regard, the at least one cartridge engagement feature 236 may be considered to have a tiered structure or tiered configuration between the inferior surface 208, a surface of the ledge 240, and the superior surface 206 of the upper plate 200, for purposes of the present disclosure.

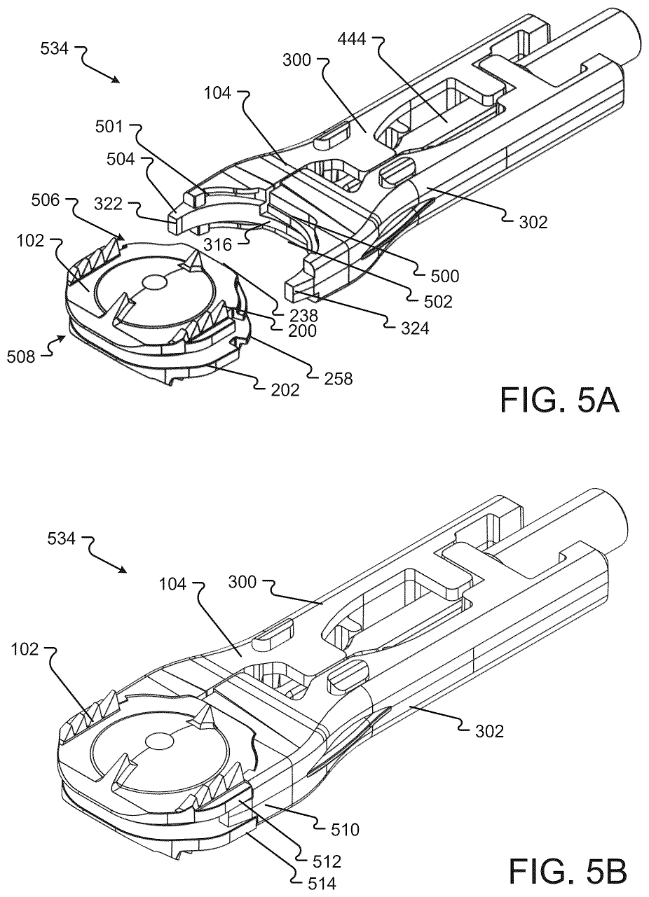

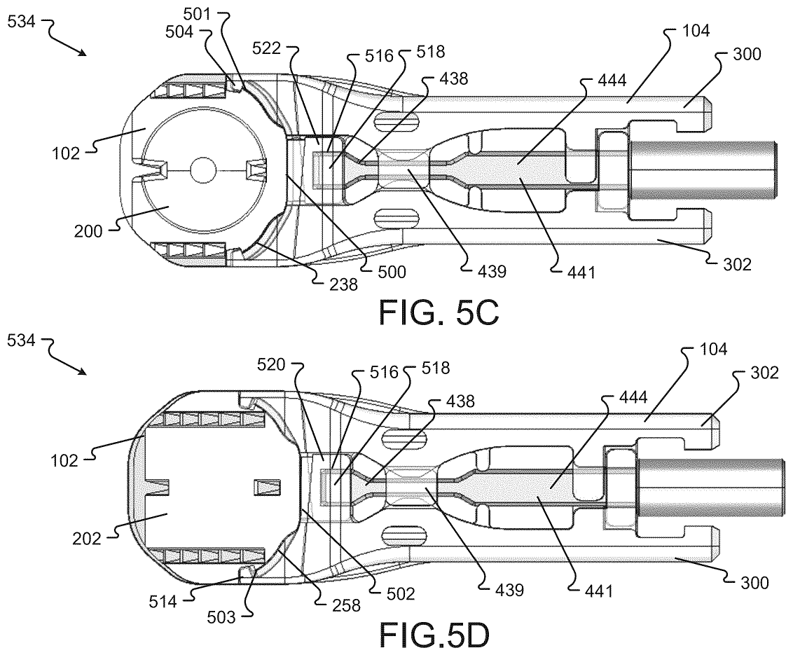

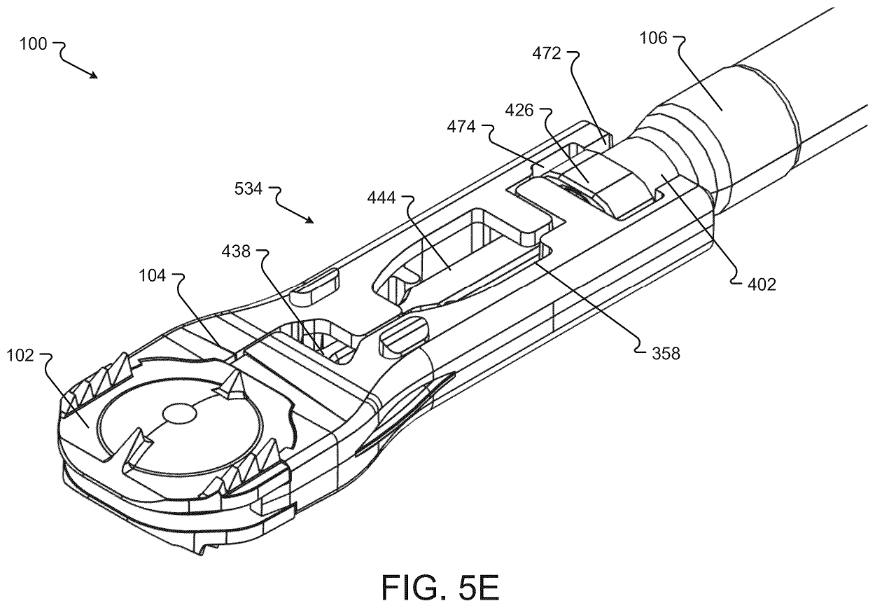

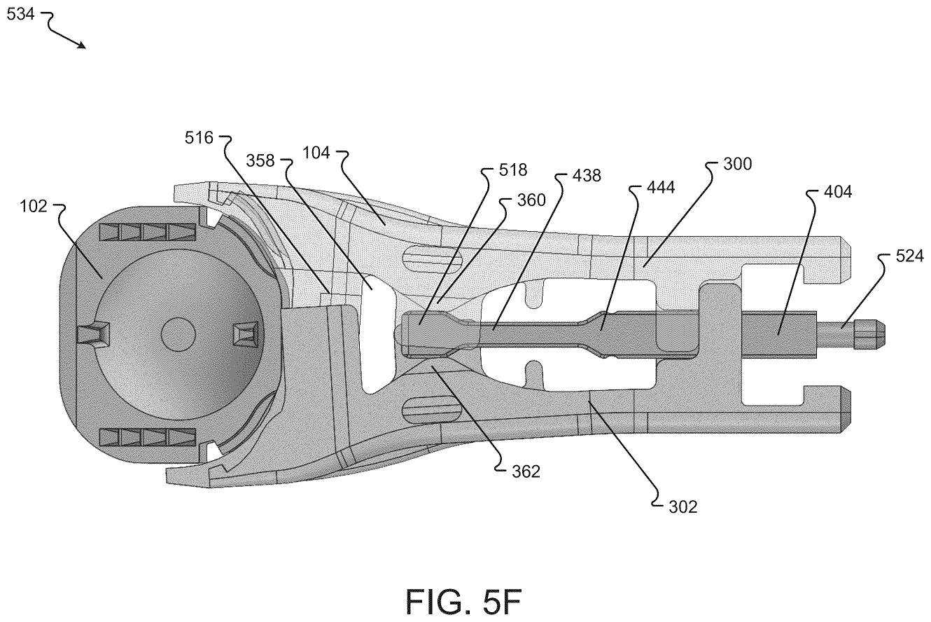

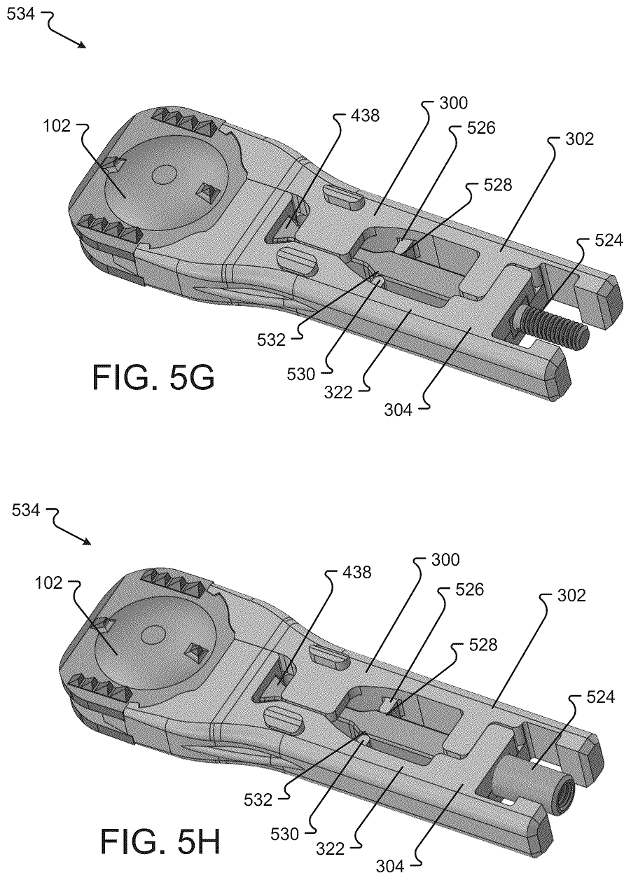

In some embodiments, as illustrated in FIG. 2E, the ledge 240 terminates at the notch 242. At least a portion of the notch 242 extends a greater amount from an exterior edge and toward a central point of the superior surface 206 than the ledge 240 into the superior surface 206 of the upper plate 200, such that one or more portions 243 of the superior surface 206 are defined by the ledge 240 on a first edge and by the notch 242 on a second edge. As illustrated in FIGS. 5A-5H, engagement of these portions 243 by the cartridge 104 may promote an increased level of securing of the prosthesis 102 within the cartridge 104 (e.g., prior to insertion into a patient at an implantation site).

In embodiments, the upper plate 200 includes at least one set of teeth. The at least one set of teeth are configured to engage a contact surface of a respective (e.g., upper) vertebral body of the adjacent vertebral bodies between which the prosthesis 102 is inserted during implantation, to prevent movement (e.g., sidewards and/or backwards movement) of the upper plate 200 following implantation. In this regard, the at least one set of teeth may be considered anchoring teeth for the upper plate 200.

For example, the at least one set of teeth includes first and second sets of teeth 244 on the substantially flat or planar portion 220 of the superior surface 206. For instance, the first and second sets of teeth 244 are aligned along the side edges 234 and flank the convex portion 218 of the superior surface 206, being proximate to respective side edges 234. It is noted, however, that the first and second sets of teeth 244 may at least partially overlap with the convex portion 218, without departing from the scope of the present disclosure. In addition, the first and second sets of teeth 244 may be positioned between the front edge 230 and the notch 242 on the superior surface 206.

It is noted that at least one tooth of the set of teeth 244 may optionally form a portion of the front edge 230, the side edge 234, and/or the rear edge 238 of the upper plate 200. For example, the side edge 234 of the upper plate 200 may extend to an upper surface of the tooth of the set of teeth 244. In addition, it is noted that a particular set of teeth 244 may at least partially align with a particular cartridge engagement feature 236. For example, the particular set of teeth 244 may at least partially align with the notch 242.

By way of another example, the at least one set of teeth includes a set of alignment teeth 246. The set of alignment teeth 246 may include at least two teeth that are aligned and positioned on the convex portion 222 of the superior surface 206. In some instances, the set of alignment teeth 246 are positioned on a midline through the front edge 230 and rear edge 238 that is spaced substantially equally between the side edges 234 of the upper plate 200. This positioning allows the set of alignment teeth 246 to be sighted with a midline of/on the upper vertebral body and/or a midline on the cartridge 104 and/or the instrument 106 during implantation.

It is noted that at least one tooth of the set of alignment teeth 246 may optionally form a portion of the front edge 230 or the rear edge 238 of the upper plate 200. For example, the chamfer 228 of the upper plate 200 may extend from the front edge 230 to an upper surface of the tooth of the set of alignment teeth 246.

In some non-limiting examples, the first and second sets of teeth 244 and/or the set of alignment teeth 246 may have teeth that are offset triangular prisms, with four sides that come to an offset point and a bottom on (or part of) the superior surface 206. The teeth of the first and second sets of teeth 244 and/or the set of alignment teeth 246 may be substantially the same size or different sizes. For example, the teeth of the first and second sets of teeth 244 and/or the set of alignment teeth 246 may change in length, width, and/or height relative to the superior surface 206 from the front edge 230 to the rear edge 238. For instance, the length, width, and/or height of the teeth of the first and second sets of teeth 244 and/or the set of alignment teeth 246 may increase from the front edge 230 to the rear edge 238. It is noted, however, that the teeth 244 and/or teeth 246 may not come to a point, but instead have sides that converge to a line or to a surface, without departing from the scope of the present disclosure.

In embodiments, the lower plate 202 includes a substantially flat or planar superior surface 210 and a substantially flat or planar inferior surface 212. A protrusion 248 may extend away from (e.g., upward) from the superior surface 210. For example, the protrusion 248 may be positioned in a substantially centered location on the lower plate 202. The protrusion 248 may range from 2-6 millimeters (mm) in diameter and may optionally be 3 mm or 5 mm in diameter.

In embodiments, the lower plate 202 includes a chamfer 250 on a front edge or leading edge 252. For example, the chamfer 250 may facilitate insertion of the prosthesis 102 into a cavity defined between the adjacent vertebral bodies during implantation. Optionally, the lower plate 202 includes a chamfer on one or more side edges 254. For example, both side edges 254 may include the chamfer. It is noted that the chamfer may facilitate insertion of the prosthesis 102 into the cavity defined between the adjacent vertebral bodies during implantation.

In embodiments, the lower plate 202 includes at least one cartridge engagement feature 256 on a rear edge or trailing edge 258. For example, the lower plate 202 includes two opposing cartridge engagement features 256 on the rear edge 258. The at least one cartridge engagement feature 256 includes a ledge 260 within the inferior surface 212 and a notch 262 through the thickness of the lower plate 202 and between the superior surface 210 and the inferior surface 212. In this regard, the at least one cartridge engagement feature 256 may be considered to have a tiered structure or tiered configuration between the superior surface 210, the ledge 260, and the inferior surface 212 of the lower plate 202, for purposes of the present disclosure.

In some embodiments, as illustrated in FIG. 2F, the ledge 260 terminates at the notch 262. At least a portion of the notch 262 extends a greater depth than the ledge 260 into the inferior surface 212 of the lower plate 202, such that one or more portions 263 of the inferior surface 212 are defined by the ledge 260 on a first edge and by the notch 262 on a second edge. As illustrated in FIGS. 5A-5H, engagement of these portions 263 by the cartridge 104 may promote an increased level of securing of the prosthesis 102 within the cartridge 104 (e.g., prior to insertion into a patient at an implantation site).

It is noted that the ledges 240, 260 and the notches 242, 262 may be symmetrical, in some instances, between the upper plate 200 and the lower plate 202 about a plane through the insert 204. In addition, it is noted that the ledges 240, 260 and the notches 242, 262 may be symmetrical, in some instances, on the upper plate 200 and the lower plate 202, respectively, about a plane through the upper plate 200 and the lower plate 202. As described in further detail with respect to the cartridge 104, this symmetrical configuration may optionally allow for the cartridge 104 to be used in multiple orientations, reducing the time necessary to prepare the system 100 for use prior to or during an operation.

In embodiments, the lower plate 202 includes at least one set of teeth. The at least one set of teeth are configured to engage a contact surface of a respective (e.g., lower) vertebral body of the adjacent vertebral bodies between which the prosthesis 102 is inserted during implantation, to prevent movement (e.g., sidewards and/or backwards movement) of the lower plate 202 following implantation. In this regard, the at least one set of teeth may be considered anchoring teeth for the lower plate 202.

For example, the at least one set of teeth includes first and second sets of teeth 264 on the inferior surface 212. For instance, the first and second sets of teeth 264 are aligned along the side edges 254. In addition, the first and second sets of teeth 264 may be positioned between the front edge 252 and the rear edge 258, such that the first and second sets of teeth 264 extend on the inferior surface 212 beyond the notches 262 from the front edge 252.

It is noted that at least one tooth of the set of teeth 264 may optionally form a portion of the front edge 252, the side edge 254, and/or the rear edge 258 of the lower plate 202. For example, the side edge 254 of the lower plate 202 may extend from the front edge 252 to a lower surface of the tooth of the set of teeth 264.

By way of another example, the at least one set of teeth includes a set of alignment teeth 266. The set of alignment teeth 266 may include at least two teeth that are aligned and positioned on the inferior surface 212. In some instances, the set of alignment teeth 266 are positioned on a midline through the front edge 252 and rear edge 258 that is spaced substantially equally between the side edges 254 of the lower plate 202. This positioning allows the set of alignment teeth 266 to be sighted with a midline of/on the lower vertebral body and/or a midline on the cartridge 104 and/or the instrument 106 during implantation.

It is noted that at least one tooth of the set of alignment teeth 266 may optionally form a portion of the front edge 252 or the rear edge 258 of the lower plate 202. For example, the chamfer 250 of the lower plate 202 may extend to a lower surface of the tooth of the set of alignment teeth 266.

The first and second sets of teeth 264 and/or the set of alignment teeth 266 may have teeth that are offset triangular prisms, with four sides that come to an offset point and a bottom on the inferior surface 212. The teeth of the first and second sets of teeth 264 and/or the set of alignment teeth 266 may be substantially the same size or different sizes. For example, the teeth of the first and second sets of teeth 264 and/or the set of alignment teeth 266 may increase in length, width, and/or height relative to the inferior surface 212 from the front edge 252 to the rear edge 258. For instance, the length and height of the teeth of the first and second sets of teeth 264 and/or the set of alignment teeth 266 may increase from the front edge 252 to the rear edge 258. It is noted, however, that the teeth 264 and/or teeth 266 may not come to a point, but instead have sides that converge to a line or to a surface, without departing from the scope of the present disclosure.

As illustrated in FIGS. 2G and 2H, the sets of teeth 244 and the sets of teeth 264 may be in corresponding sets. For example, the corresponding sets may be offset (e.g., a different distance from respective side edges 234, 254), due to the positioning of the teeth 244, 264 relative to the respective notches 242, 262. However, it should be understood that the corresponding sets may be substantially aligned (e.g., a substantially same distance from respective side edges 234, 254) and not be offset, without departing from the scope of the present disclosure.

In some embodiments, the sets of teeth 244 on the upper plate 200 may have fewer teeth than the corresponding sets of teeth 264 on the lower plate 202. For example, the sets of teeth 244 may each have four teeth, while the corresponding sets of teeth 264 may each have five or six teeth. The additional number of teeth in the sets of teeth 264 may cause the sets of teeth 264 to substantially extend between the front edge 252 and the rear edge 258 of the lower plate 202, such that at least a portion of teeth in the sets of teeth 264 form a portion of the front edge 252 and/or the rear edge 258 of the lower plate 202.

In some instances, at least one tooth of the sets of teeth 244, 246 may be shorter in height from the superior surface 206 than an apex of the convex portion 218 of the dome region of the superior surface 206 of the upper plate 200. In other instances, at least one tooth of the sets of teeth 264, 266 may be taller from the inferior surface 212 of the lower plate 202 than the teeth of the sets of teeth 244, 246 from the superior surface 206 of the upper plate 200.

In embodiments, as illustrated in FIGS. 2C-2D and 2K-2L, at least a portion of the superior surface 214 of the insert 204 includes a convex and dome-shaped region 268. For example, the convex region 268 may be the entire superior surface 214. Alternatively, the convex region 268 may be a portion of the superior surface 214 that is at least partially surrounded by a substantially flat or planar ring or flange portion having a decreased height relative to an apex of the convex region 268. For example, the ring or flange portion may limit rotation in flexion/extension and lateral/bending directions. Where the superior surface 214 includes the ring or flange portion, a transition between the convex region 268 and the substantially flat or planar ring may be sloped and/or without a step-like change in height. Alternatively, the transition between the convex region 268 and the substantially flat or planar ring may have a step-like change in height.

In embodiments, at least a portion of the inferior surface 216 of the insert 204 is substantially flat or planar. A recess 270 is positioned at a substantially centered location in the inferior surface 216. The recess 270 is configured to receive the protrusion 248 on the lower plate 202. For example, the recess 270 may range from 2-6 millimeters (mm) in diameter and may optionally be 3 mm or 5 mm in diameter. It is noted that the recess 270 may be substantially similar in diameter to the protrusion 248, or may be larger than the protrusion 248 to allow for increased adjustment of the insert 204 relative to the lower plate 202, without departing from the scope of the present disclosure.

Optionally, one or more grooves 272 may extend from an endwall 274 of the insert 204 in the inferior surface 216. For example, the one or more grooves 272 may extend from the endwall 274, across the inferior surface 216, and to the groove 272. The one or more grooves 272 may assist in the flow of fluids (e.g., that form within the recess 270) out of the recess 270 and into the cavity defined between the upper plate 200/lower plate 202 (and thus the cavity defined between the adjacent vertebral bodies in which the prosthesis 102 is implanted). The one or more grooves 272 may additionally or alternatively assist in the movement of the insert 204 relative to the lower plate 202, by adding fluid flow between the insert 204 and the lower plate 202 and reducing the possibility the inferior surface 216 of the insert 204 sticks to the superior surface 210 of the lower plate 202.

In some instances, the protrusion 248 and/or the recess 270 has a depth that is at least a height of the endwall 274. In other instances, the protrusion 248 and/or the recess 270 has a depth greater than the height of the endwall 274.

Although the insert 204 is illustrated as having a cylinder with a substantially circular cross-section and that is capped with a dome-shaped portion of a sphere, it is contemplated that any cross-section is feasible for use in the insert 204, where a sufficient amount of motion is provided by the insert 204 (and thus the prosthesis 102) to the patient in which the prosthesis 102 is implanted.

As illustrated in FIGS. 2I-2J, one contemplated natural state for the prosthesis 102 includes having the upper plate 200 offset slightly forward of, and tilted slightly toward, the lower plate 202. For example, the upper plate 200 may be offset from the lower plate 202 by approximately 0.5 mm. In addition, the upper plate 200 may be tilted toward the lower plate 202 by approximately 3°. It is noted, however, that this is only an exemplary state, and that other natural states are contemplated, without departing from the scope of the present disclosure.

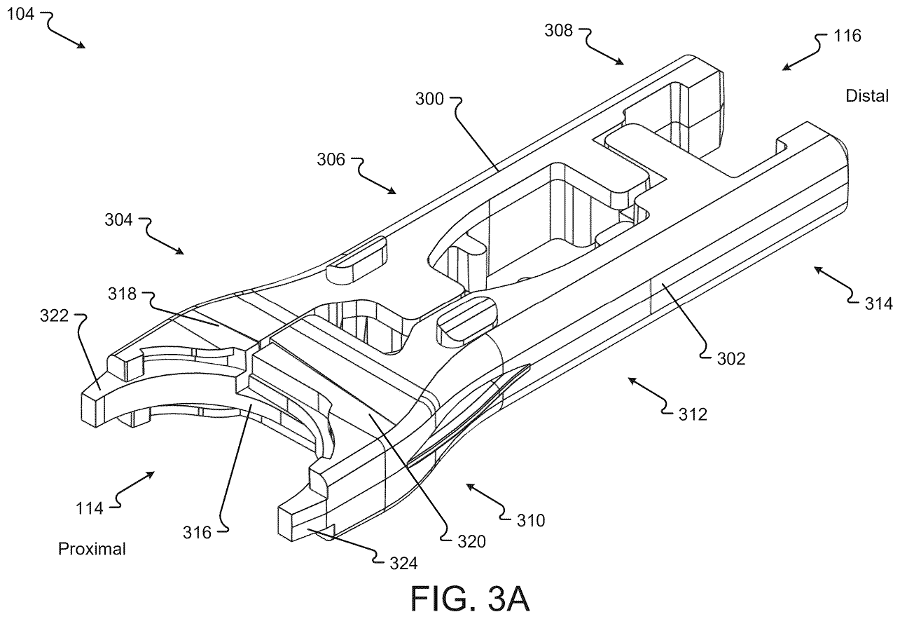

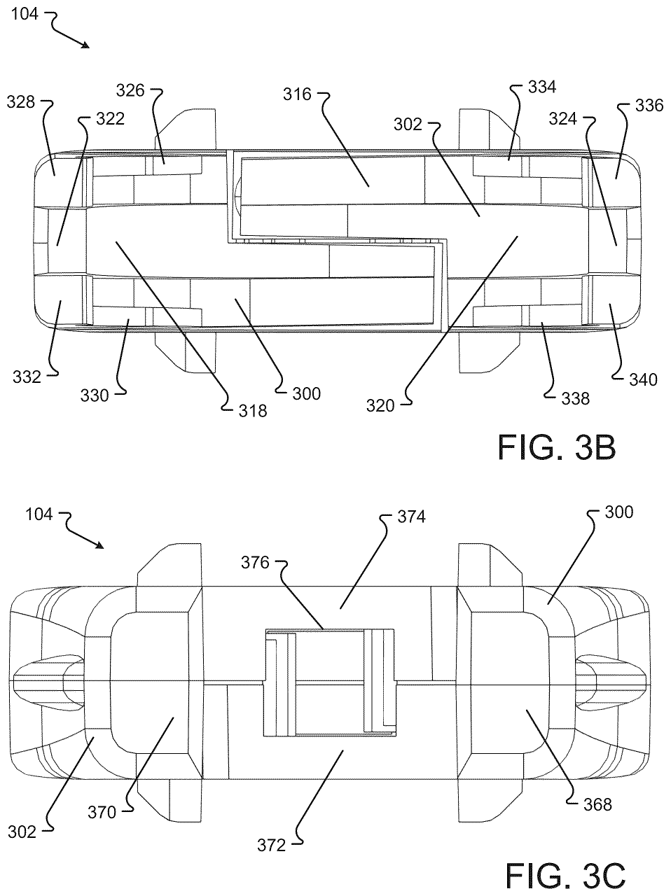

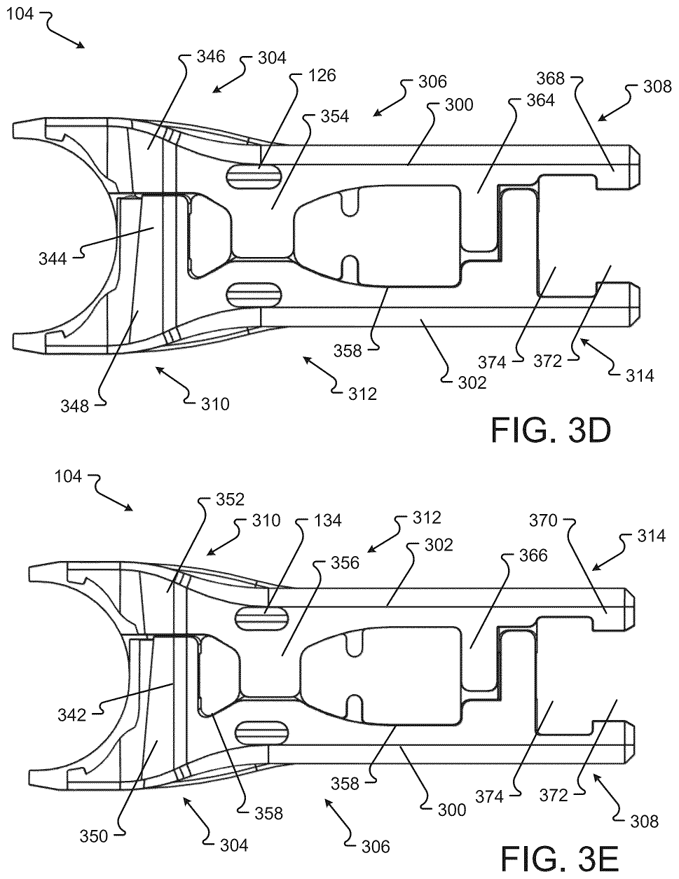

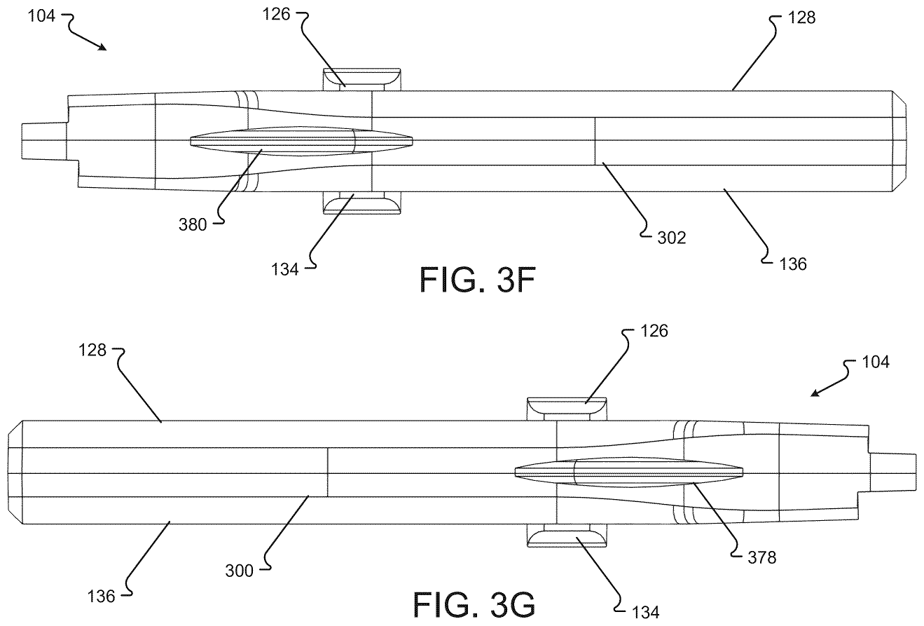

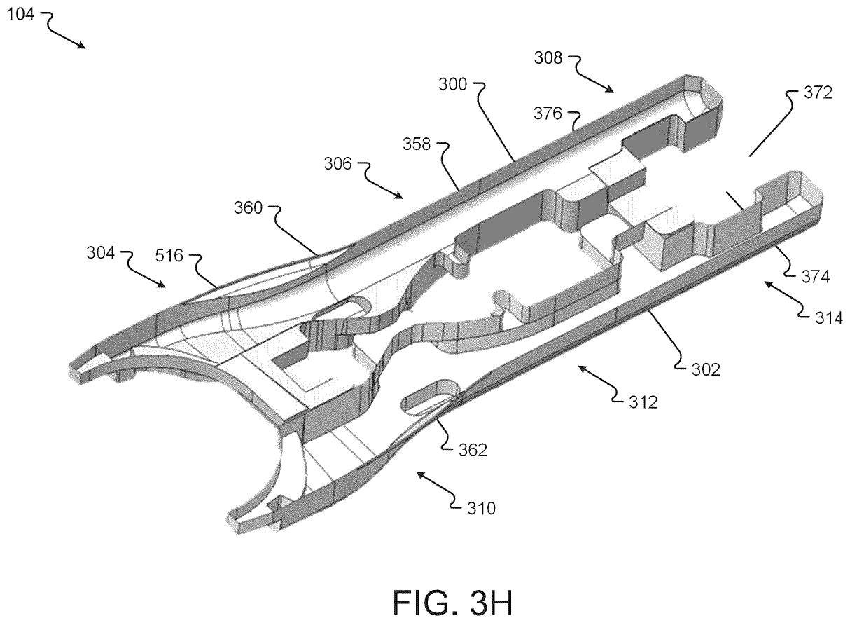

FIGS. 3A-3G in general illustrate the cartridge 104 of the system 100, in accordance with one or more embodiments of the present disclosure. It should be understood that any embodiment of the cartridge 104 illustrated in FIGS. 1A-1G may be directed to the cartridge 104 illustrated in FIGS. 3A-3G, and vice versa, without departing from the scope of the present disclosure.

In embodiments, the cartridge 104 includes a first arm 300 and a second arm 302. The first arm 300 has a proximal section 304, a middle section 306, and a distal section 308. The second arm 302 has a proximal section 310, a middle section 312, and a distal section 314. The proximal section 304 of the first arm 300 and the proximal section 310 of the second arm 302 form the proximal end 112 of the cartridge 104. The distal section 308 of the first arm 300 and the distal section 314 of the second arm 302 form the distal end 114 of the cartridge 104.

In embodiments, the proximal end 112 of the cartridge 104 includes a receiver 316, formed from a first hand 318 on the first arm 300 and a second hand 320 on the second arm 302. The first hand 318 includes a first finger 322. The second hand 320 includes a second finger 324.

In embodiments, the receiver 316 includes one or more engagement features configured to engage the prosthesis 102. For example, the receiver 316 engages the prosthesis 102 on at least one of the rear edge 238 and/or the side edges 234 of the upper plate 200. In addition, the receiver 316 engages the prosthesis 102 on at least one of the rear edge 258 and/or the side edges 254 of the lower plate 202.

In embodiments, the engagement features of the cartridge 104 include shelves that correspond to ledges on the prosthesis 102. For example, the first hand 318 includes an upper shelf 326 that is positionable over a first ledge 240 of the upper plate 200 and an upper tab 328 that is positionable within the first notch 242 of the upper plate 200. By way of another example, the first hand 318 includes a lower shelf 330 that is positionable under a first ledge 260 of the lower plate 202 and a lower tab 332 that is positionable within a first notch 262 of the lower plate 202.

By way of another example, the second hand 320 includes an upper shelf 334 that is positionable over a second ledge 240 of the upper plate 200 and an upper tab 336 that is positionable within a second notch 242 of the upper plate 200. By way of another example, the second hand 320 includes a lower shelf 338 that is positionable under a second ledge 260 of the lower plate 202 and a lower tab 340 that is positionable within a second notch 262 of the lower plate 202.

It is noted that, in some configurations, a symmetry between the fingers 322, 324, the shelves 326, 330, 334, 338 and the ledges 240, 260, and the tabs 328, 332, 336, 340 and the notches 242, 262, allow for the cartridge 104 engage the prosthesis 102 in either a first orientation or a second orientation, reducing the time needed for assembly of the prosthesis 102 and the cartridge 104 prior to or during an operation. However, this is possible only where the natural state is not the exemplary offset and tilted configuration of the upper plate 200 and the lower plate 202, as described previously herein. Where the natural state is the exemplary offset and tilted configuration, the shelves 326, 330, 334, 338 and the tabs 328, 332, 336, 340 may be positioned on the first hand 318 and the second hand 320 accordingly.

As illustrated in FIGS. 3D and 3E, the proximal section 304 of the first arm 300 includes a proximal crossmember 342, and the proximal section 310 of the second arm 302 includes a proximal crossmember 344. In some embodiments, the proximal crossmember 342 and the proximal crossmember 344 are each of a thickness that is less than a total thickness of the respective first arm 300 and second arm 302. For example, the thicknesses of the crossmembers 342, 344 may be such that, when the cartridge 104 is assembled and the crossmembers 342, 344 are overlapping, an upper surface 346 of the first arm 300 and an upper surface 348 of the second arm 302 (e.g., that together form the upper surface 128 of the cartridge 104) are substantially flush or planar. In addition, the thicknesses of the crossmembers 342, 344 may be such that, when the cartridge 104 is assembled and the crossmembers 342, 344 are overlapping, a lower surface 350 of the first arm 300 and a lower surface 352 of the second arm 302 (e.g., that together form the lower surface 136 of the cartridge 104) are substantially flush or planar.

It is noted that the upper wedges 126 are on the upper surface 346 of the first arm 300 and the upper surface 348 of the second arm 302. In addition, the lower wedges 134 are on the lower surface 350 of the first arm 300 and the lower surface 352 of the second arm 302.

In embodiments, the middle section 306 of the first arm 300 includes a middle crossmember 354, and the middle section 312 of the second arm 302 includes a middle crossmember 356. In some embodiments, the middle crossmember 354 and the middle crossmember 356 are each of a thickness that is less than a total thickness of the respective first arm 300 and second arm 302. For example, the thicknesses of the crossmembers 354, 356 may be such that, when the cartridge 104 is assembled and the crossmembers 354, 356 are overlapping, the upper surface 346 of the first arm 300 and the upper surface 348 of the second arm 302 (e.g., that together form the upper surface 128 of the cartridge 104) are substantially flush or planar. In addition, the thicknesses of the crossmembers 354, 356 may be such that, when the cartridge 104 is assembled and the crossmembers 354, 356 are overlapping, the lower surface 350 of the first arm 300 and the lower surface 352 of the second arm 302 (e.g., that together form the lower surface 136 of the cartridge 104) are substantially flush or planar.

In some embodiments, a cavity 358 may be at least partially defined within the proximal sections 304, 310 and/or at least partially defined within the middle sections 306, 312 of the first arm 300 and the second arm 302, respectively. A portion of the instrument 106 (e.g., a locking pin 444 with an expansion bit 438) may be inserted to engage with the cartridge 104 and cause the cartridge 104 to release the prosthesis 102. For example, the crossmember 354 may include a release tab 360, and the crossmember 356 may include a release tab 362. As described in detail further herein, the portion of the instrument 106 (e.g., the locking pin 444 with the expansion bit 438) engages the release tabs 360, 362 to cause the cartridge 104 to release the prosthesis 102 after the prosthesis 102 is implanted at the implantation site.

It is noted that the cartridge 104 may only require one of the release tabs 360, 362 to disengage the cartridge 104 from the prosthesis 102 via interaction with the portion of the instrument 106, without departing from the scope of the present disclosure.

In some configurations, the cartridge 104 and the locking pin 444 with the expansion bit 438 may be pre-packaged together as a kit or assembly. In additional configurations, the prosthesis 102, the cartridge 104, and the locking pin 444 with the expansion bit 438 may be pre-packaged together as a kit or assembly. In further configurations, the prosthesis 102 and the cartridge 104 may be pre-packaged together as a kit or assembly. This pre-packaging may reduce the possibility of interference with (e.g., damaging, misaligning, and the like) the assembly of the prosthesis prior to insertion into the patient, while promoting sterility and the interchangeability of the assembly and a trial implant 600. For example, different trial implants 600 may be tried with the same instrument 106, and then a kit or assembly including the prosthesis 102 that matches the trial implant 600, the cartridge 104, and/or the locking pin 444 with the expansion bit 438 may be installed within the instrument 106 for implantation.

In embodiments, the distal section 308 of the first arm 300 includes a distal crossmember 364, and the distal section 314 of the second arm 302 includes a distal crossmember 366. In some embodiments, the distal crossmember 364 and the distal crossmember 366 are each of a thickness that is less than a total thickness of the respective first arm 300 and second arm 302. For example, the thicknesses of the crossmembers 364, 366 may be such that, when the cartridge 104 is assembled and the crossmembers 364, 366 are at least partially overlapping, the upper surface 346 of the first arm 300 and the upper surface 348 of the second arm 302 (e.g., that together form the upper surface 128 of the cartridge 104) are substantially flush or planar. In addition, the thicknesses of the crossmembers 364, 366 may be such that, when the cartridge 104 is assembled and the crossmembers 364, 366 are overlapping, the lower surface 350 of the first arm 300 and the lower surface 352 of the second arm 302 (e.g., that together form the lower surface 136 of the cartridge 104) are substantially flush or planar.

As illustrated in FIGS. 3B-3C and 3F-3G, in some embodiments the upper surface 128 and the lower surface 136 of the cartridge 104 are respectively substantially flat or planar along the entire length of the cartridge 104, aside from the wedges 126, 134 that extend from the respective surfaces 128, 136. In some instances, a total height of the upper wedges 126 from the upper surfaces 346, 348 is equal to or less than a height of the tallest tooth of the sets of teeth 244, 246 on the upper plate 200 (e.g., as illustrated in FIGS. 5A-5H). In other instances, a total height of the lower wedges 134 from the upper surfaces 346, 348 is equal to or less than a height of the tallest tooth of the sets of teeth 264, 266 on the lower plate 202 (e.g., as illustrated in FIGS. 5A-5H). This allows for a reduced size of implantation site in the patient, as the surfaces 346, 348, 350, 352 with respective wedges 126, 134 do not extend outside of a height envelope defined by the respective sets of teeth 244, 246, 264, 266.

The distal section 308 additionally includes a first tail 368, and the distal section 314 additionally includes a second tail 370. The space between the first tail 368 and the second tail 370 defines a slot 372 of the distal end 114 of the cartridge 104. In addition, the crossmembers 364, 366 and the tails 368, 370 define a cavity 374 of the distal end 114 of the cartridge 104.

In some embodiments, the cavity 374 has a greater width than the slot 372. As described in detail further herein, a portion of the instrument 106 (e.g., a coupler 426) may be inserted within the slot 372 and re-oriented once located within the cavity 374 to mate with the cavity 374. When in the first orientation, the portion of the instrument 106 (e.g., the coupler 426) may be removed from the cavity 374 and the slot 372. When in the second orientation, the portion of the instrument 106 (e.g., the coupler 426) is retained within the cavity 374 by the slot 372, due to the mating between the coupler 426 and the cavity 374.

The crossmembers 364, 366 may each be configured to collectively define an opening 376 in the distal end of the cartridge 104. In some embodiments, the opening 376 provides access for a portion of the instrument 106 (e.g., the locking pin 444 with the expansion bit 438) into the cavity 358 for engagement with the release tabs 360, 362. In embodiments where the locking pin 444 with the expansion bit 438 is pre-packaged with the cartridge 104 (and the prosthesis 102), the expansion bit 438 may be at least partially positioned within the cavity 374, such that the coupler 426 may receive the expansion bit 438 when inserted through the slot 372.

In some embodiments, the first arm 300 may include a support member 378 between the proximal section 304 and the middle section 306. In addition, the second arm 302 may include a support member 380 between the proximal section 310 and the middle section 312. In some instances, the support members 378, 380 are dimensioned to be no wider than the width of the proximal end 112 of the cartridge 104. This reduces the total footprint width of the combined prosthesis 102 and cartridge 104, reducing the width of the implantation site needed during the operation.

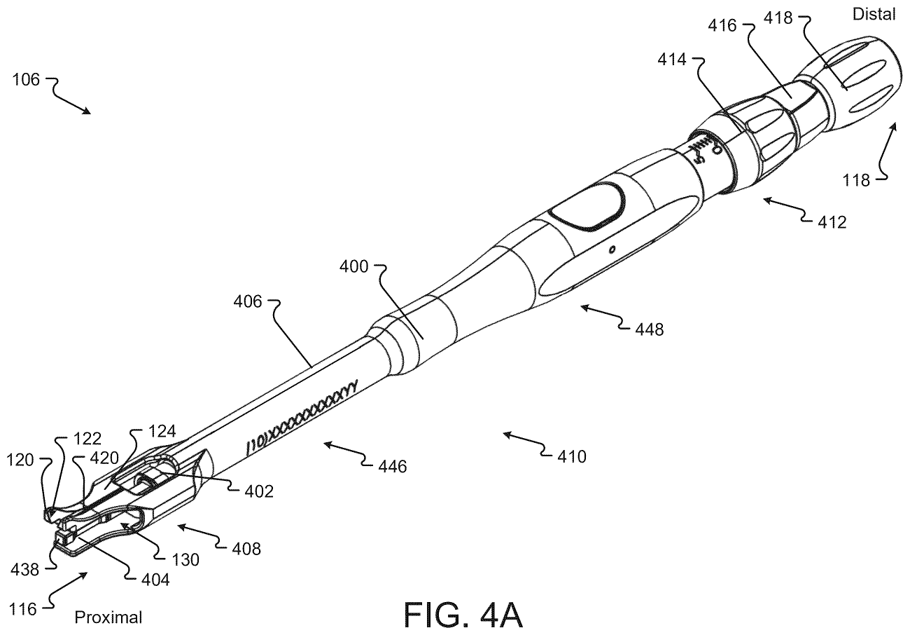

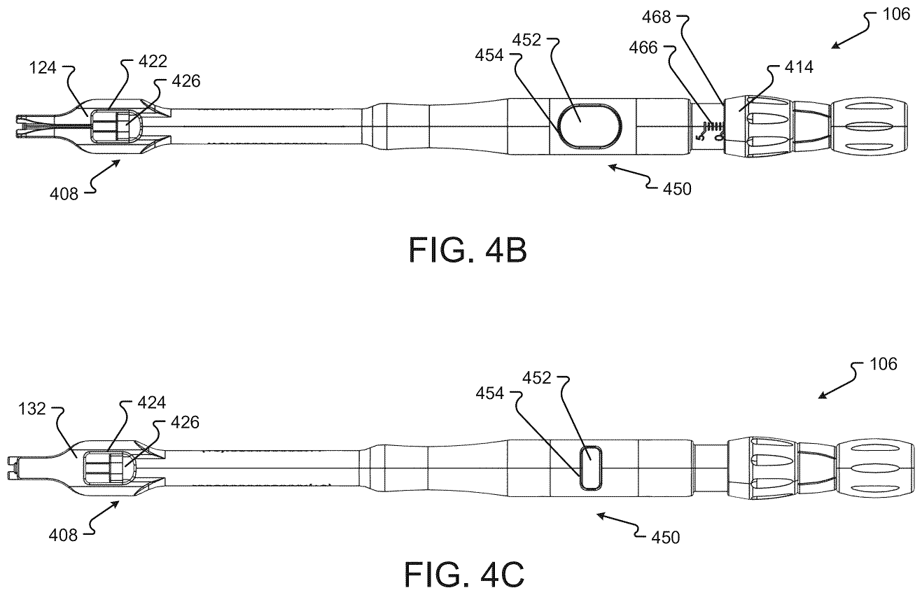

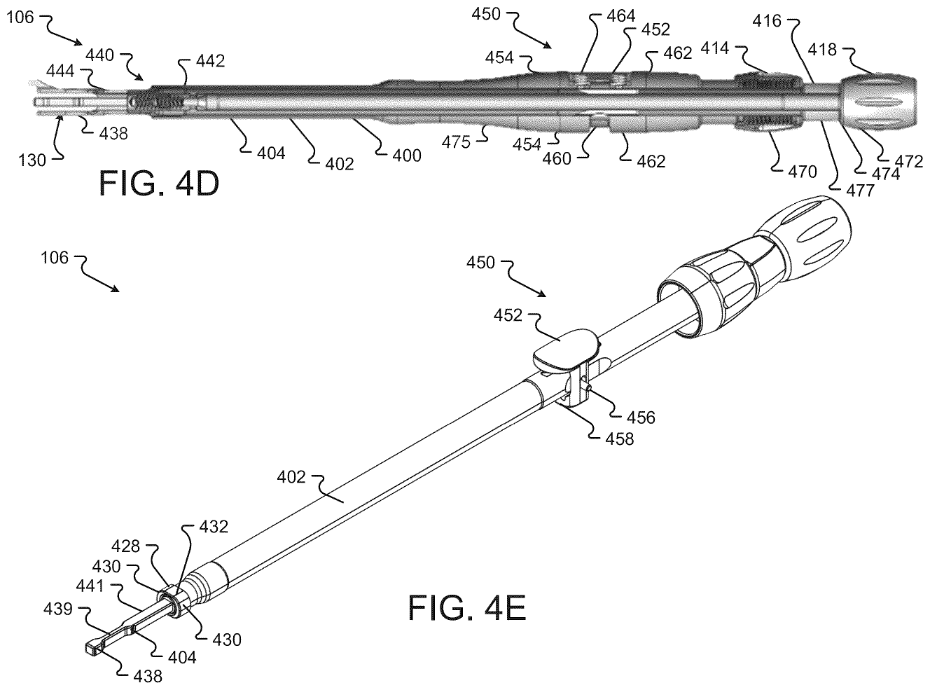

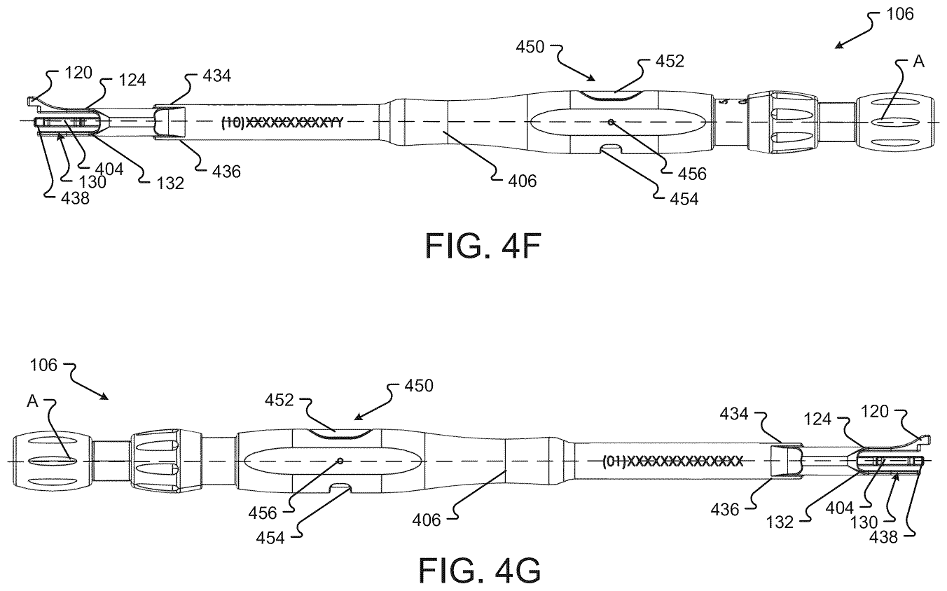

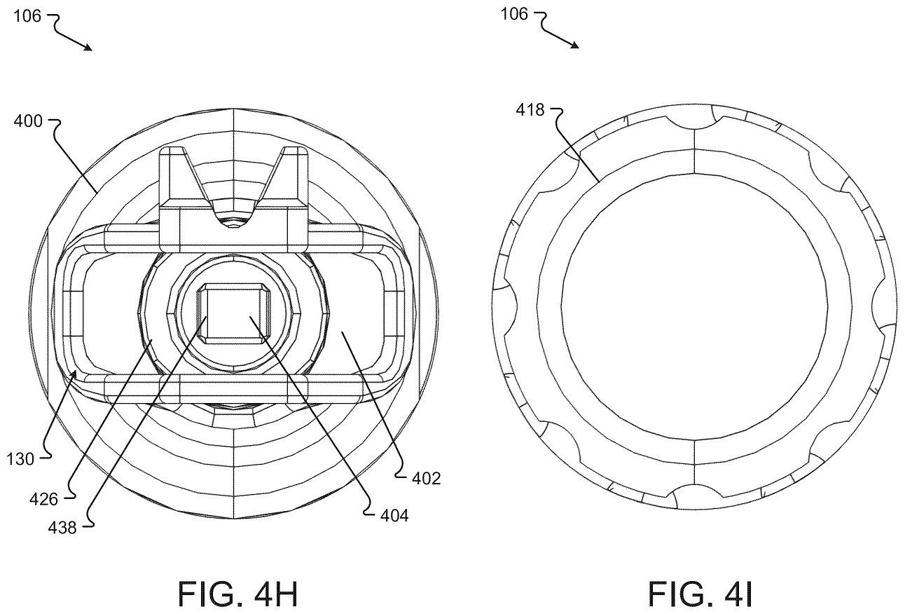

FIGS. 4A-4I in general illustrate the instrument 106 of the system 100, in accordance with one or more embodiments of the present disclosure. It should be understood that any embodiment of the instrument 106 illustrated in FIGS. 1A-1G may be directed to the instrument 106 illustrated in FIGS. 4A-4I, and vice versa, without departing from the scope of the present disclosure.

In embodiments, the instrument 106 includes an outer housing 400, a locking shaft 402 (or middle shaft), and a release driver 404 (or inner shaft). The outer housing 400 includes a body 406 that extends from the proximal end 116 to the distal end 118 of the instrument 106. The body 406 includes a proximal section 408, a middle section 410, and a distal section 412.

The outer housing 400 is connected to an actuator 414. The actuator 414 (e.g., proximate to the distal end 118) of the instrument 106 causes a displacement of depth stops 120 on the upper tab 124 of the body 406 of the instrument 106 relative to the cartridge 104 when the cartridge 104 is inserted within the opening 130 of the instrument 106. For example, the depth stops 120 may be displaced along a longitudinal axis A through the instrument 106 via actuation of the actuator 414, to assist in the positioning of the prosthesis 102 with the cartridge 104 between adjacent vertebral bodies, as described in detail further herein.

In addition, the locking shaft 402 passes through the outer housing 400 and is connected to an actuator 416. For instance, the actuator 416 (e.g., proximate to the distal end 118) of the instrument 106 causes the locking shaft 402 to receive and engage the cartridge 104 when the cartridge 104 is inserted into the opening 130 of the instrument 106, as described in detail further herein.

Further, the release driver 404 passes through the locking shaft 402 and is connected to an actuator 418. The actuator 418 (e.g., proximate to the distal end 118) of the instrument 106 actuates the release driver 404 to cause the cartridge 104 to disengage from the prosthesis 102, as described in detail further herein.

Although separate exemplary actuators 414, 416, 418 are illustrated, it should be understood that the instrument 106 may be configured such that the same actuator 414, 416 and/or 418 performs multiple of the described operations, without departing from the scope of the present disclosure.

In the proximal section 408, the instrument 106 includes the upper tab 124 with the channel 122 defined by at least one depth stop 120, and the lower tab 132. The upper tab 124 includes an alignment feature 420 that aligns with complementary features on the prosthesis 102 and/or the cartridge 104. For example, the alignment feature 420 may be a groove, a recess, a protrusion, a mark (e.g., laser-etched, drawn, an applied sticker, or the like), colors, and/or patterns or another feature that aligns with complementary protrusions (e.g., teeth) or indentations on the prosthesis 102 and/or the cartridge 104, and/or protrusions or indentations on a proximate vertebral body. For instance, the shape of the channel 122 and/or the location of the alignment feature 420 may correspond to a midline of the adjacent vertebral bodies between which the prosthesis 102 is being implanted. In this regard, a user (e.g., a surgeon, or other medical professional) may utilize the combination of the alignment feature 420 and the complementary features on the cartridge 104 and/or the prosthesis 102, along with any optional marks on the adjacent vertebral bodies, to sight the prosthesis 102 during insertion between the adjacent vertebral bodies at the implantation site and/or after during post-operative monitoring.

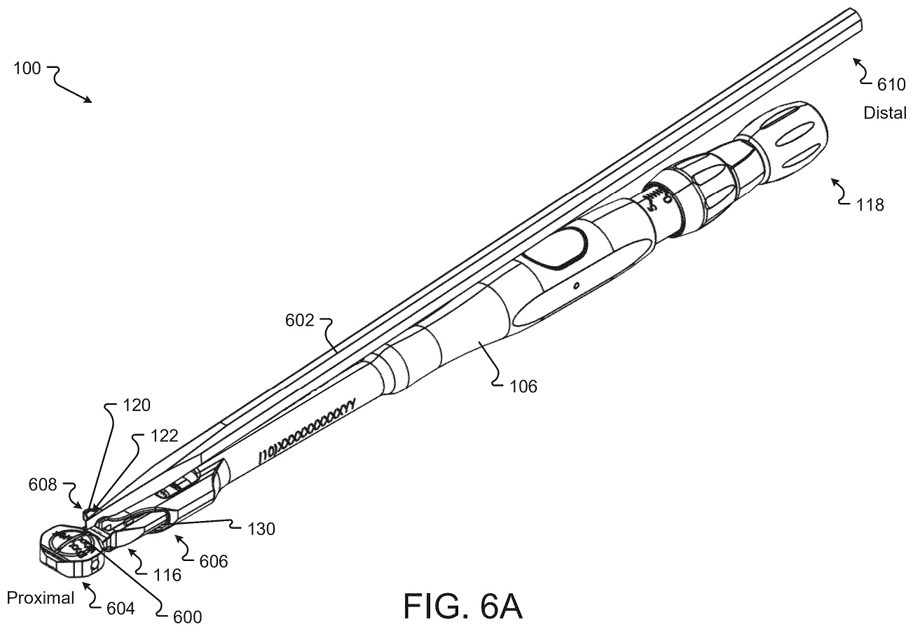

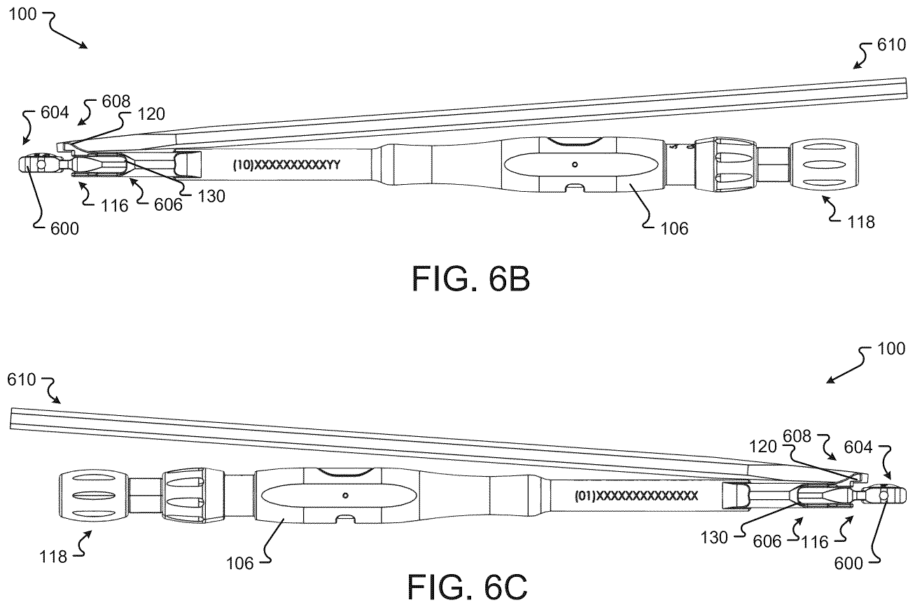

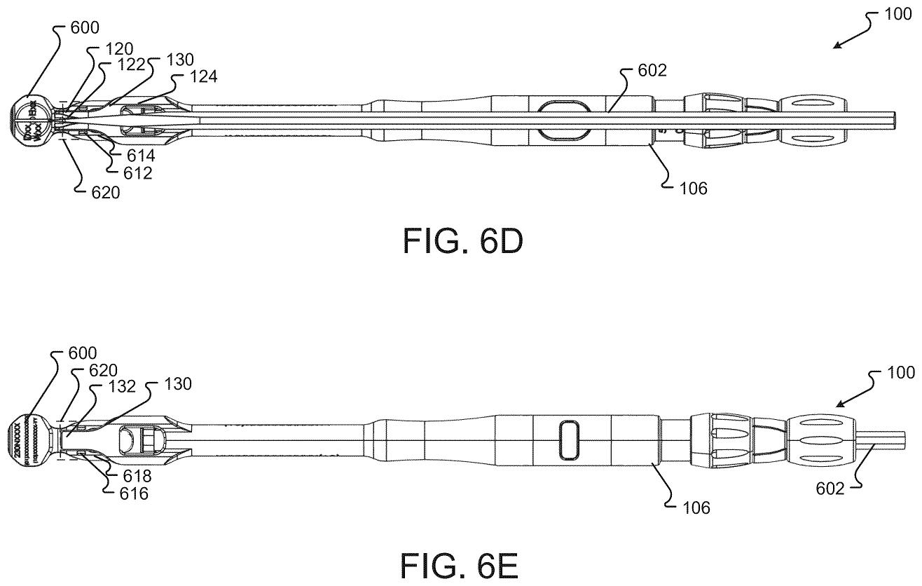

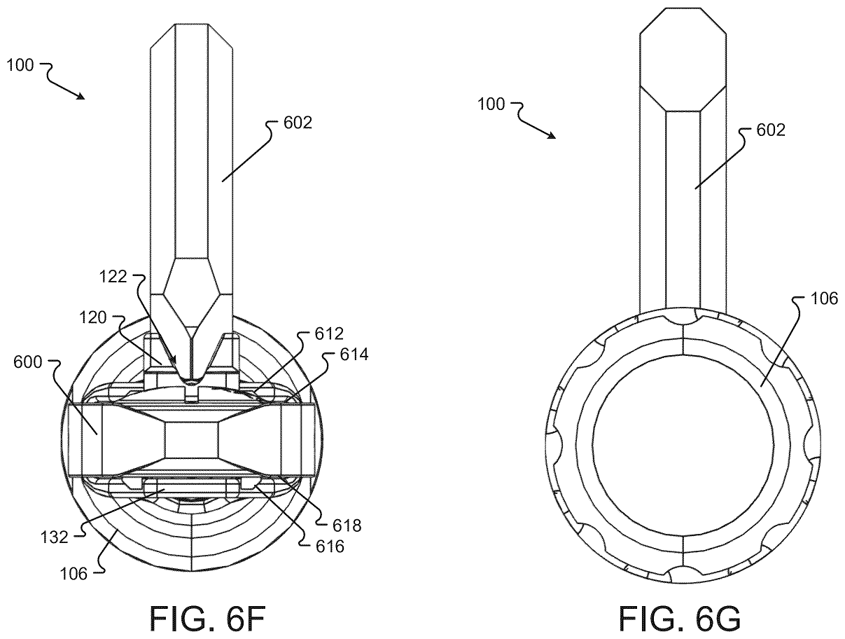

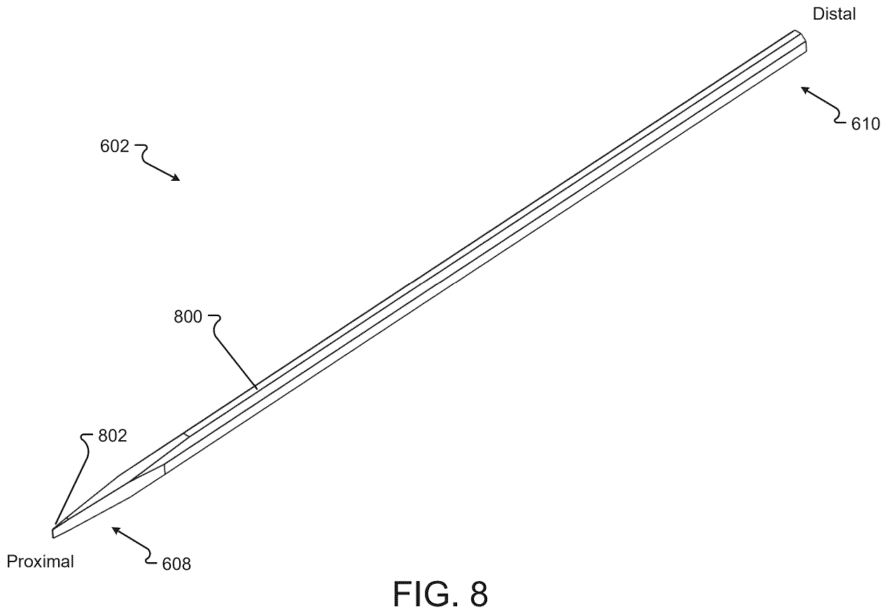

In some embodiments, such as where the alignment feature 420 is a groove or recess, the alignment feature 420 can optionally assist in positioning an osteotome 602 (e.g., as illustrated in FIGS. 6A-6G and 8) within the channel 122 between the depth stops 120 on the upper tab 124 of the instrument 106, as described in detail further herein. For example, this may be beneficial where the proximal end 116 of the instrument 106 is not visible as it is inserted within the implantation site, such that visual confirmation of the appropriate position of the osteotome 602 within the channel 122 is not available.

As illustrated in FIGS. 4B and 4C, the proximal section 408 of the body 406 includes at least one of a first window 422 in the upper tab 124 of the instrument and a second window 424 in the lower tab 132 of the instrument 106. A coupler 426 of the locking shaft 402 is visible within the windows 422, 424 (e.g., being positioned at a proximal end of the locking shaft 402), and the mating of the coupler 426 to the cartridge 104 and/or a trial implant 600 is visible through the windows 422, 424.

In some non-limiting examples, as illustrated in FIG. 4E, the coupler 426 and the locking shaft 402 form a T-shape, with locking shaft 402 for the long leg of the T-shape and the coupler 426 including a middle section 428 and a pair of lobes 430 for the short leg of the T-shape. A width of the coupler 426 is such that the coupler 426 may fit through a width of the slot 372 at the distal end 114 of the cartridge 104 when the coupler 426 is in a first orientation. In addition, a length of the coupler 426 is such that the coupler 426 may be retained within the cavity 374 when the coupler 426 is in a second orientation, as the length of the coupler 426 is greater than the width of the slot 372.

In additional non-limiting examples, the locking shaft 402 may include a slot 432 that is decreased in diameter as compared to the length of the coupler 426. For instance, the slot 432 may correspond to the tails 368, 370 of the cartridge 104, to allow for rotation of the locking shaft 402 within the slot 372 at the distal end 114 of the cartridge 104 when the coupler 426 is positioned within the cavity 374 at the distal end 114 of the cartridge 104.

In further non-limiting examples, a thickness of the coupler 426 is approximately a thickness of the distal end 114 of the cartridge 104, such that the coupler 426 and the distal end 114 of the cartridge 104 are approximately equal in height when the coupler 426 is in the second orientation. It is noted that the window 422 may allow for the rotation between the first orientation and the second orientation, such as where the length of the coupler 426 may be greater than the thickness of the cartridge 104 and/or a thickness between an upper surface 434 and a lower surface 436 of the proximal section 408 of the body 406.

In general, the coupler 426 may have any cross-section that allows for an insertion of the coupler 426 into the slot 372 and the cavity 374 at the distal end 114 of the cartridge 104 when the coupler 426 is in the first orientation, and for a retaining of the coupler 426 within the slot 372 and the cavity 374 at the distal end 114 of the cartridge 104 when the coupler 426 is in the second orientation. As such, the above examples should not be interpreted as limiting on the present disclosure.

In embodiments, the release driver 404 extends through the locking shaft 402 and exits proximal to the opening 130 of the instrument 106. The release driver 404 has an expansion bit 438. The expansion bit 438 is configured to engage with the cartridge 104 to cause the cartridge 104 to release the prosthesis 102 when the release driver 404 is actuated, as described in detail further herein.

In some embodiments, at least a portion of the release driver 404 may be removable from and couplable to the instrument 106. For example, as illustrated in FIG. 4D, the release driver 404 includes an interlocking assembly 440 to couple an actuator portion 442 (e.g., that includes a shaft with the actuator 418) with a locking pin 444 (e.g., that includes the expansion bit 438 (as a proximal portion), a middle portion 439, and a distal portion 441, where the actuator portion 442 is coupled to the distal portion 441). In some instances, the interlocking assembly 440 includes complementary threading. However, any interlocking assembly 440 is contemplated as being appropriate to disengage the actuator portion 442 from the locking pin 444.

In other embodiments, the release driver 404 may be a single-piece configuration with the actuator 418 and the expansion bit 438. The release driver 404 may be installed within or insertable into the locking shaft 402. The release driver 404 may engage the cartridge 104 once inserted into the opening 130 of the instrument 106, including to cause the cartridge 104 to release the prosthesis 102 after implantation of the prosthesis 102.

Where the release driver 404 is separated into the actuator portion 442 and the locking pin 444, it is noted that the locking pin 444 (with expansion bit 438) may be provided with an assembly 534 (e.g., as illustrated in FIGS. 5A-5H) including the cartridge 104 (and optionally the prosthesis 102), when the assembly 534 is inserted into the opening 130 of the instrument 106. Alternatively, the actuator portion 442 and the locking pin 444 may be provided to the user in a kit as separate components, but assembled within the instrument 106 prior to the assembly 534 being inserted into the opening 130 of the instrument 106.

In embodiments, the middle section 410 of the body 406 includes a first portion 446 proximate to the proximal section 408 of the body 406 and at least a second portion 448 proximate to the distal section 412 of the body 406. The first portion 446 may have a first width or diameter, and the at least a second portion 448 may have at least a second diameter. For example, the first width or diameter of the first portion 446 may be less than or equal to a total width between opposite side surfaces of the proximal section 408, to reduce a need for an increased footprint to accommodate the first portion 446 at the implantation size where the entire proximal section 408 is inserted into the patient during implantation. By way of another example, the at least a second width or diameter of the at least a second portion 448 may be greater than the first width or diameter of the first portion 446. For instance, the at least a second portion 448 may be contoured to conform to a user's hand and may include at least a second width or diameter that transitions from the lesser first diameter or diameter of the first portion 446 to an increased diameter for the user's hand.

It is noted, however, that the at least a second portion 448 may be able to receive a separate and attachable grip portion that is contoured to conform to a user's hand. In addition, it should be understood that the middle section 410 may be substantially similar in width or diameter along the entire length of the middle section 410, without departing from the scope of the present disclosure.

In some embodiments, as illustrated in FIGS. 4B-4E, the middle section 410 (e.g., in the second portion 448) includes a lock assembly 450. The lock assembly 450 includes a toggle 452 positioned within at least one slot 454 in the outer housing 400. At least one pin 456 secures the toggle 452 within the at least one slot 454 in the outer housing 400. In one non-limiting example, the toggle 452 includes an aperture 458 through which the locking shaft 402 is inserted, and the toggle 452 extends through multiple slots 454 in the outer housing 400.

The toggle 452 includes a protrusion 460 that engages a recess 462 on the locking shaft 402 when a biasing element 464 provides a force on the toggle 452, locking the locking shaft 402 and preventing rotation of the locking shaft 402. For example, rotation of the locking shaft 402 may be locked after insertion of the cartridge and prosthesis assembly 534 into the opening 130 of the instrument 106. When a user applies a force to the toggle 452, the at least one biasing element 464 may be overcome and the toggle 452 to may disengage from the recess 462 to allow for rotation of the locking shaft 402.

In embodiments, the distal section 412 of the body 406 includes the actuators 414, 416, 418. For example, the actuator 414 for the depth stops 120 is proximal of the actuator 416 for the locking shaft 402, and the actuator 416 for the locking shaft 402 is proximal of the actuator 418 for the release driver 404.

The body 406 includes a plurality of markers 466 that indicate the positioning of the depth stops 120 (e.g., relative to the cartridge 104 when inserted into the opening 130 of the instrument 106) based on a rotation of the actuator 414. For example, rotation of the actuator 414 causes an indicator 468 on the actuator 414 to align with a particular marker of the plurality of markers 466. For example, the plurality of markers 466 may represent a range from 0 to 15 millimeters (mm), and optionally from 0 to 5 mm. By way of another example, the plurality of markers 466 may be in increments between 0 and 2 mm for a full rotation of the actuator 1514, including optionally 1 mm or 0.5 mm increments for a full rotation of the actuator 1514. It is noted that the markers 466 and the indicator 468 may be visual indicators, with an edge of the actuator 414 and/or a mark (e.g., laser-etched, drawn, an applied sticker, or the like), colors, and/or patterns on the actuator 414 being the indicator 468 that aligns with a particular marker 466. In addition, it is noted that the indicator 468 may be a window in the actuator 414 through which the markers 466 are visible.

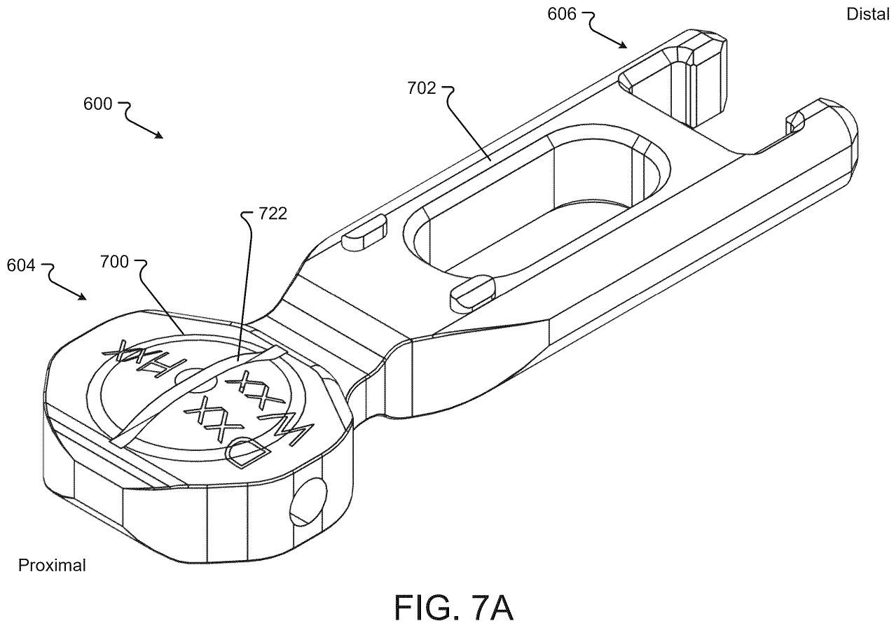

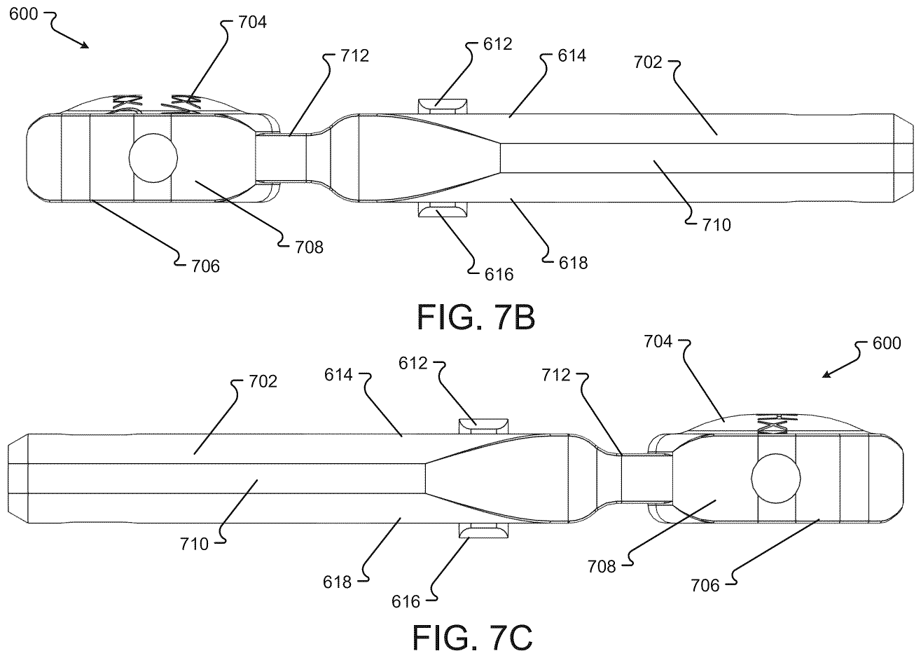

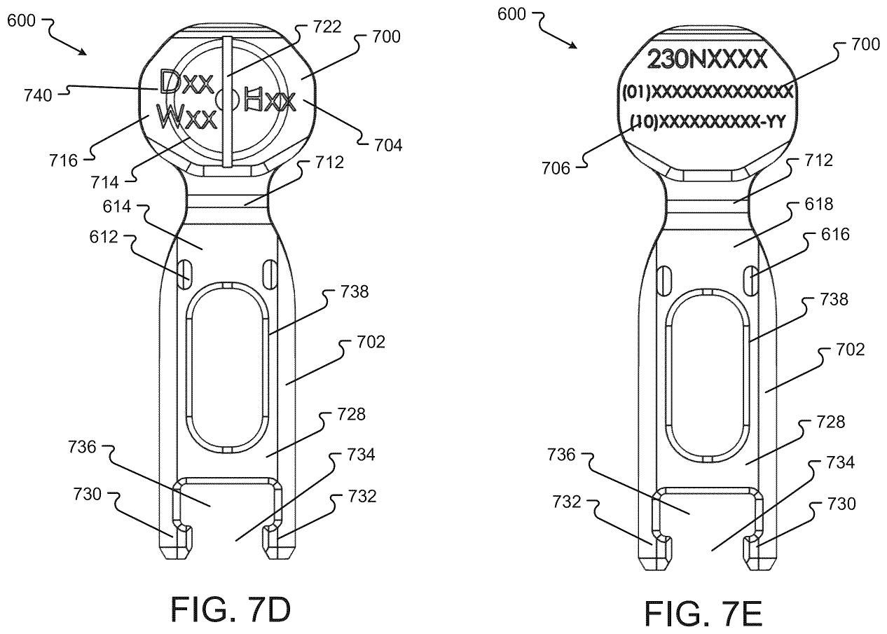

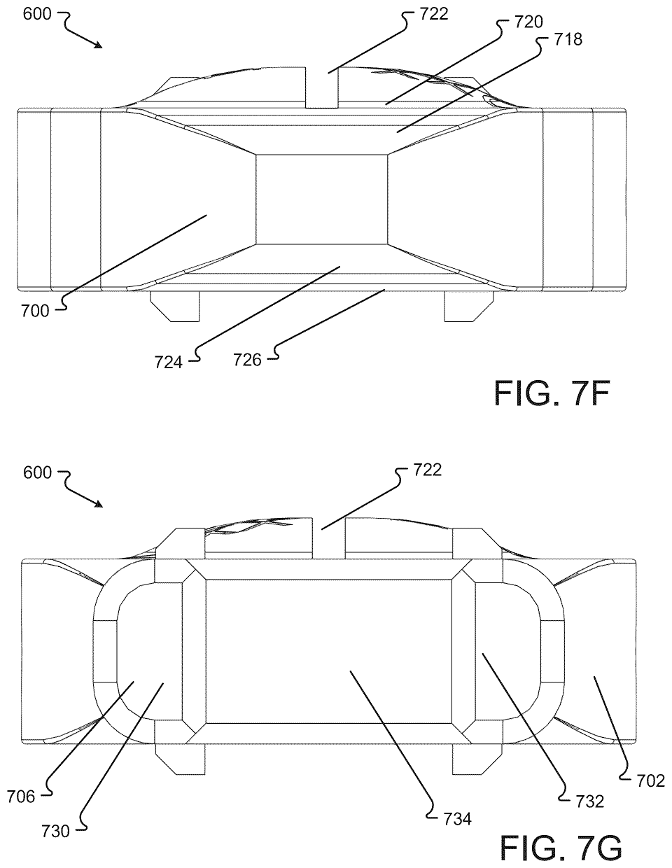

Rotation of the actuator 414 causes the depth stops 120 to move by engaging complementary threading 470 in the actuator 414 and on the body 406 of the outer housing 400. For example, increased engagement between the threading 470 in the actuator 414 and on the body 406 may shorten the length of the outer housing 400 by pulling the body 406 into the actuator 414, while the locking shaft 402 holds the received cartridge 104 (or a trial implant 600, as illustrated in FIGS. 6A-7G and described in detail further herein) in place within the instrument 106. Doing so sets the adjustment for the depth stop 120, which is determined by a user based on the depth needed for insertion of the prosthesis 102 between the adjacent vertebral bodies.

In some embodiments, such as where the release driver 404 is separated into the actuator portion 442 and the locking pin 444, the actuator portion 442 with the actuator 418 is removable from, and insertable into, an aperture 472 in a distal end surface 474 of the actuator 416. For example, the user may use the instrument 106 with only the actuators 414 and 416 when inserting the prosthesis 102 with the cartridge 104 (e.g., that has the locking pin 444 inserted within the cartridge 104). The user may then insert the actuator portion 442 of the release driver 404 into the aperture 472 to engage the locking pin 444, to cause the cartridge 104 to release the placed prosthesis 102. Doing so is beneficial, as it may reduce the possibility the prosthesis is advertently released with the release driver 404 prior to correct placement at the implantation site.

In embodiments, the actuator portion 442 includes a proximal member 475 and a distal shaft 477. For example, the proximal member 475 may be coupled to the locking pin 444, and the distal shaft 477 may be in communication with the actuator 418. The proximal member 475, the distal shaft 477, and the actuator 418 may be formed as a single integrated unit. Alternatively, one or more of the proximal member 475, the distal shaft 477, and/or the actuator 418 may be separately formed and coupled to the remainder of the components.

In some configurations, the proximal member 475 is a smaller diameter than the distal shaft 477. The graduated diameters may be beneficial to prevent shearing of the actuator portion 442 when a torque is applied to the actuator 418, while still allowing for a smaller diameter along the length of the instrument 106. However, it is contemplated that a diameter of the proximal member 475 and the distal shaft 477 may be substantially the same, such that two sections of the actuator portion 442 may be considered one component, without departing from the scope of the present disclosure.

FIGS. 5A-5H in general illustrate the engaging of the prosthesis 102 by the cartridge 104, and the engaging of the combined prosthesis 102/cartridge 104 by the instrument 106, of the system 100, in accordance with one or more embodiments of the present disclosure. It should be understood that any embodiment of the prosthesis 102 illustrated in FIGS. 1A-1G and 2A-2L, the cartridge 104 illustrated in FIGS. 1A-1G and 3A-3G, and/or the instrument 106 illustrated in FIGS. 1A-1G and 4A-4I may be directed to the prosthesis 102, the cartridge 104 and/or the instrument 106 illustrated in FIGS. 5A-5H, and vice versa, without departing from the scope of the present disclosure.

In FIG. 5A, the cartridge 104 (e.g., including, optionally, an installed locking pin 444 with the expansion bit 438 of the release driver 404) is prepared to receive the prosthesis 102. In FIGS. 5B-5D, the prosthesis 102 is positioned within the receiver 316 of the cartridge 104. In embodiments, the prosthesis 102, the cartridge 104, and optionally the locking pin 444 including the expansion bit 438 of the release driver 404 is considered an assembly 534, for purposes of the present disclosure.

In general, as illustrated in FIGS. 5B-5D, the receiver 316 includes a first edge 500 with one or more portions 501 that conforms to the rear edge 238 of the upper plate 200, and a second edge 502 with one or more portions 503 that conforms to the rear edge 258 of the lower plate 202. The edges 500, 502 with respective portions 501, 503 of the cartridge 104 and the corresponding edges 238, 258 of the prosthesis 102, in combination of the engagement of the shelves 326, 330, 334, 338 and the ledges 240, 260, and the tabs 328, 332, 336, 340 and the notches 242, 262 of the cartridge 104 and the prosthesis 102, creates a more secure connection between the cartridge 104 and the prosthesis 102 during implantation.

The portions 501 of the first edge 500 of the receiver 316 that are contoured to conform to the rear edge 238 of the upper plate 200, and specifically that are contoured to conform to the engagement portions 243 along the rear edge 238, may promote an increased level of securing of the prosthesis 102 within the cartridge 104 (e.g., prior to insertion into a patient at an implantation site). Similarly, the portions 503 of the second edge 502 of the receiver 316 that are contoured to conform to the rear edge 258 of the lower plate 202, and specifically that are contoured to conform to the engagement portions 263 along the rear edge 258, may promote an increased level of securing of the prosthesis 102 within the cartridge 104 (e.g., prior to insertion into a patient at an implantation site).

Due to the contouring of the first finger 322 and the second finger 324, a side surface 504 of the first finger 322 of the first arm 300 is substantially flush or planar with the side surfaces 506 and 508 of the upper plate 200 and the lower plate 202, respectively. In addition, a side surface 510 of the second finger 324 of the second arm 302 is substantially flush or planar with opposite side surfaces 512 and 514 of the upper plate 200 and the lower plate 202, respectively. This reduces the total footprint width of the combined prosthesis 102 and cartridge 104 assembly, reducing the width of the implantation site needed during the operation.

Due to the contouring of the shelves 326, 330 and the ledges 240, 260, and the tabs 328, 332 and the notches 242, 262, the upper surface 128 of the cartridge 104 and the superior surface 206 of the upper plate 200 are substantially flush or planar. In addition, due to the contouring of the shelves 334, 338 and the ledges 240, 260, and the tabs 336, 340 and the notches 242, 262, the lower surface 136 of the cartridge 104 and the inferior surface 212 of the lower plate 202 are substantially flush or planar. This reduces the total footprint height of the combined prosthesis 102 and cartridge 104 assembly, reducing the height of the implantation site needed during the operation.

FIGS. 5C and 5D illustrate the engagement of the locking pin 444 with the cartridge 104. It is noted that the arms 300, 302 of the cartridge 104 are shown as transparent structures in FIGS. 5C and 5D to better illustrate the interface between the cartridge engagement features 236, 256 of the prosthesis 102 and the receiver 316 of the cartridge 104. In embodiments, the cartridge 104 includes an end cavity 516 (e.g., also shown in FIG. 3H) that is dimensioned to receive and engage with a proximal end 518 of the release driver 404 (e.g., a proximal end 518 of the locking pin 444, or the expansion bit 438 of the locking pin 444). The end cavity 516 may be positioned within the first arm 300 and/or within the second arm 302. In some non-limiting examples, the end cavity 516 is defined by surfaces in both the first arm 300 and the second arm 302. For instance, the first arm 300 may include a proximal portion 520 that is proximate to (or is an integrated portion of) the hand 318 of the first arm 300, and the second arm 302 may include a proximal portion 522 that is proximate to (or is an integrated portion of) the hand 320 of the second arm 302. In this instance, interior surfaces defined within the proximal portion 520 of the first arm 300 (e.g., as shown in FIGS. 3H and 5F) and interior surfaces within the proximal portion 522 of the second arm 302 together define the end cavity 516 within the cartridge 104.

The end cavity 516 is dimensioned to receive the proximal end 518 of the release driver 404 (e.g., the expansion bit 438 of the locking pin 444). In some configurations, the positioning of the proximal end 518 within the end cavity 516 promotes the cartridge 104 remaining closed, such that the prosthesis 102 remains secured within the cartridge 104 prior to actuation of the release driver 404.

As illustrated in FIG. 5E, the coupler 426 of the instrument 106 is retained within the cavity 374 and the slot 372 when the coupler 426 is in the second orientation. The coupler 426 is shaped to pass through the slot 372 into the cavity 374 in the distal end 114 of the cartridge 104 when in a first orientation, and is shaped to be retained within the cavity 374 when in a second orientation. For example, the orientation of the coupler 426 may be adjusted through actuation of the actuator 416. For instance, rotation of the actuator 416 causes the coupler 426 to rotate from the first orientation to the second orientation. In this regard, the locking shaft 402 can receive the slot 372 and the cavity 374 at the distal end 114 of the cartridge 104 when the cartridge 104 is inserted into the opening 130 of the instrument 106 and then can be rotated to the second orientation to engage the cavity 374, causing the cartridge 104 to be retained within the opening 130 of the instrument 106. It is noted that the connection of the coupler 426 of the locking shaft 402 to the cavity 374 and the slot 372 at the distal end 114 of the cartridge 104 may be visible through the windows 422, 424 of the instrument 106, but that the visibility is not required for the system 100 to operate (e.g., as the visibility may not be possible, depending on a depth the proximal end 116 of the instrument 106 with the prosthesis 102 and the cartridge 104 is inserted into a patient at the implantation site).

In addition, the locking pin 444 is within the cavity 358 in the cartridge 104. In some instances, the expansion bit 438 of the locking pin 444 is inserted into the cavity 358 during insertion of the cartridge 104 into the opening 130 of the outer housing 400. In other instances, such as where the locking pin 444 with the expansion bit 438 of the release driver 404 is provided within the cavity 358 as part of the prosthesis and cartridge assembly 534, the actuator portion 442 of the release driver 404 passes through the locking shaft 402 and engages the provided locking pin 444 within the cartridge 104, after insertion of the prosthesis and cartridge assembly 534 into the opening 130 of the outer housing 400. It is noted that the outer housing 400 with opening 130 is not illustrated in FIG. 5C for purposes of clarity only.

As illustrated in FIG. 5F, the cartridge 104 has released the prosthesis 102. It is noted that the first arm 300 of the cartridge 104 is shown as a transparent structure in FIG. 5F to better illustrate the disengagement of the cartridge 104 and the prosthesis 102. Actuation of the release driver 404 causes the first arm 300 and the second arm 302 to expand outward via engagement of the release tabs 360, 362 by the expansion bit 438, as a width between the release tabs 360, 362 is less than a width of the expansion bit 438 (and a width of the end cavity 516 which initially housed the expansion bit 438), releasing the prosthesis 102 once the prosthesis 102 is inserted into the cavity between the adjacent vertebral bodies. For example, where the actuator portion 442 connects to the locking pin 444 via the interlocking assembly 440, engagement of the components 442, 444 via the interlocking assembly 440 causes the proximal end 518 including the locking pin 444 (e.g., with the expansion bit 438) to be pulled in a distal direction, which moves the expansion bit 438 within the cavity 358 such that the proximal end 518 actuates in the cartridge 104 from a retracted first position within the end cavity 516 to an expanded second position in which the proximal end 518 engages the release tabs 360, 362 and expands the cartridge 104. As illustrated in at least FIG. 5F, the release tabs 360, 362 and the sidewalls of the proximal end 518 may have complementary contouring to reduce the possibility of binding (and non-actuation of the arms 300, 302 in general) when the locking pin 444 is pulled in the distal direction.

It is noted that FIG. 5F illustrates an optional engagement feature 524 of the interlocking assembly 440 to couple the locking pin 444 to the actuator portion 442. FIGS. 5G and 5H illustrate variations of the engagement feature 524 of the interlocking assembly 440 that is on the locking pin 444. In particular, FIG. 5G illustrates a male threaded stud for the engagement feature 524, and FIG. 5H illustrates a female threaded bushing for the engagement feature 524. It is noted that the remainder of the instrument 106, including the locking shaft 402 with the coupler 426 through which the locking pin 444 is inserted, has been removed from FIGS. 5F-5H for purposes of clarity only.

In some embodiments, the cartridge 104 includes interlocking components that engage with the release driver 404 (e.g., on the locking pin 444 with the expansion bit 438), such that the release driver 404 (e.g., the expansion bit 438) is held within the cartridge 104 (e.g., during insertion into the opening 130 of the instrument 106). For example, the first arm 300 of the cartridge 104 includes a locking tab 526 that engages a first locking recess 528 on the release driver 404 (e.g., on the locking pin 444 with the expansion bit 438). By way of another example, the second arm 302 of the cartridge 104 includes a locking tab 530 that engages a second locking recess 532 on the release driver 404 (e.g., on the locking pin 444 with the expansion bit 438).

When the release driver 404 is actuated via the actuator 418, the locking recesses 528, 532 will disengage from the respective locking tabs 526, 530. In some instances, this occurs prior to the proximal end 518 of the expansion bit 438 engaging the release tabs 360, 362, as illustrated in FIG. 5F.

It is noted that the cartridge 104 may only require one of the locking tabs 526, 530 and the locking pin 444 may only require one of the locking recesses 528, 532 to hold the locking pin 444 within the cavity 358 of the cartridge 104, without departing from the scope of the present disclosure.

It is noted that portions of the instrument 106 have been removed from FIGS. 5A-5H for purposes of clarity only.

FIGS. 6A-6G in general illustrate the intervertebral disc prosthesis system 100 with a trial implant 600, in accordance with one or more embodiments of the present disclosure. It should be understood that any embodiment of the system 100 illustrated in FIGS. 1A-1G may be directed to the system 100 illustrated in FIGS. 6A-6G, and vice versa, without departing from the scope of the present disclosure.

The system 100 in FIGS. 6A-6G includes a trial implant 600 in place of the prosthesis 102 and the cartridge 104, and optionally an osteotome 602. In particular, the instrument 106 and the trial implant 600 may first be used to select a corresponding cartridge and prosthesis assembly 534, and then the trial implant 600 may be replaced by the cartridge and prosthesis assembly 534 to implant the prosthesis 102 with the cartridge 104. Reference will be made to each of the components 600, 602 in detail further herein. In addition, any and/or all of the components 600, 602 may include additional components as described in detail further herein, which are each considered components of the system 100.

As illustrated in FIGS. 6A-6G, the trial implant 600 includes a proximal end 604 and a distal end 606. The osteotome 602 includes a proximal end 608 and a distal end 610. The proximal end 604 of the trial implant 600 is insertable into a cavity defined between adjacent vertebral bodies. The distal end 606 of the trial implant 600 is additionally insertable into the opening 130 at the proximal end 116 of the instrument 106.

The proximal end 608 of the osteotome 602 is insertable into the channel 122 defined by the at least one depth stop 120 on the upper tab 124 of the instrument 106 (e.g., at the proximal end 116 of the instrument 106). For example, the channel 122 may guide the proximal end 608 of the osteotome 602 to mark a midline on a vertebral body (e.g., an upper vertebral body of the adjacent set between which the prosthesis 102 is to be implanted). For instance, the distal end 610 of the osteotome 602 may be engaged with by a user (e.g., such as a surgeon or other medical professional) to mark the midline on the vertebral body.

The upper tab 124 of the instrument 106 is configured to engage at least one upper wedge 612 on an upper surface 614 of the trial implant 600, to assist in aligning the trial implant 600 within the opening 130 in the proximal end 116 of the instrument 106. For example, the upper surface 614 may include two upper wedges 612 that flank the upper tab 124 of the instrument 106 during insertion of the trial implant 600 into the opening 130 of the instrument 106.

In other embodiments, the lower tab 132 of the instrument 106 (e.g., at the proximal end 116 of the instrument 106) is configured to engage at least one lower wedge 616 on a lower surface 618 of the trial implant 600, to assist in aligning the trial implant 600 within the opening 130 in the proximal end 116 of the instrument 106. For example, the lower surface 618 may include two lower wedges 616 that flank the lower tab 132 of the instrument 106 during insertion of the trial implant 600 into the opening 130 of the instrument 106.

It is noted that the trial implant 600 may include the upper wedges 612 and/or the lower wedges 616, either individually or in combination, without departing from the scope of the present disclosure.

FIGS. 7A-7G in general illustrate the trial implant 600, in accordance with one or more embodiments of the present disclosure. It should be understood that any embodiment of the trial implant 600 illustrated in FIGS. 6A-6G may be directed to the trial implant 600 illustrated in FIGS. 7A-7G, and vice versa, without departing from the scope of the present disclosure.

In embodiments, the trial implant 600 has a head 700 at the proximal end 604. The trial implant 600 includes a body 702 at the distal end 606.

In embodiments, the head 700 is a static size with a pre-defined depth (Dxx), width (Wxx), and/or height (Hxx). In other embodiments, the head 700 is adjustable in one or more of depth, width, and/or height (e.g., via actuation of the release driver 404 following the securing of the trial implant 600 within the opening 130 of the instrument 106 via the locking shaft 402). In general, the trial implant 600 is configured to approximate the dimensions of the cartridge 104 and a particular prosthesis 102 having a pre-defined depth, width, and/or height, so as to correctly interface with the instrument 106 and so as to correctly size a cavity between adjacent vertebral bodies for the particular prosthesis 102.

In embodiments, the head 700 includes a superior surface 704, an inferior surface 706, and one or more sidewalls 708. The body 702 includes the upper surface 614 with the upper wedges 612, the lower surface 618 with the lower wedges 616, and one or more sidewalls 710.

In embodiments, the head 700 and the body 702 are connected via a transition region 712. For example, at least a portion of the transition region 712 may be substantially equal to a width of the head 700 and/or the body 702. By way of another example, at least a portion of the transition region 712 may be narrower in width than the head 700 and/or the body 702. It is noted that the head 700 and the body 702 may be a single integrated component. Alternatively, the head 700 and the body 702 may be coupled together (e.g., at the transition region 712), such that the head 700 is interchangeable with another head 700 on the body 702, without departing from the scope of the present disclosure.

In embodiments, the superior surface 704 of the head 700 includes a dome region. In general, the dome region is shaped to contour to conform to a dome region of a particular prosthesis 102, which is optionally convex and dome-shaped on the superior surface 206 of the upper plate 200 of a particular prosthesis 102. For instance, the superior surface 704 of the trial implant 600 may be contoured to conform to a substantially spherical portion for the convex dome shape.

For example, the dome region may include a convex portion 714 at least partially surrounded by a substantially flat or planar portion 716 in the superior surface 704. The transition between the convex portion 714 and the substantially flat or planar portion 716 may be sloped and/or without a step-like change in height. Alternatively, the transition between the convex portion 714 and the substantially flat or planar portion 716 may have a step-like change in height.

In embodiments, the head 700 includes a chamfer 718 (or rounded portion) on a front edge or leading edge 720 of the superior surface 704. For example, the chamfer 718 may facilitate insertion of the trial implant 600 into the cavity defined between the adjacent vertebral bodies during trialing.

In embodiments, the superior surface 704 of the head 700 includes an alignment feature 722 (e.g., within the convex portion 714) that optionally aligns with complementary features on the instrument 106. For example, the alignment feature 722 may be a groove, a recess, a protrusion, a mark (e.g., laser-etched, drawn, an applied sticker, or the like), colors, and/or patterns or another feature that aligns with complementary alignment features on the instrument 106 (e.g., such as the channel 122). For instance, the alignment feature 420 and/or the alignment feature 722 may correspond to a midline of the adjacent vertebral bodies (e.g., as created by the osteotome 602) between which a prosthesis 102 is implanted. In this regard, a user (e.g., a surgeon, or other medical professional) may utilize the combination of the alignment feature 722 and the complementary features on the instrument 106 to sight the trial implant 600 during insertion into the cavity defined between the adjacent vertebral bodies at the implantation site during trialing.

In embodiments, the inferior surface 706 is substantially flat or planar. For example, the inferior surface 706 of the trial implant 600 may be contoured to conform to a substantially flat or planar portion of the inferior surface 212 of the lower plate 202 of a particular prosthesis 102.

In embodiments, the head 700 includes a chamfer 724 (or rounded portion) on a front edge or leading edge 726 of the inferior surface 706. For example, the chamfer 724 may facilitate insertion of the trial implant 600 into the cavity defined between the adjacent vertebral bodies during trialing.

It is contemplated that the superior surface 704 and the inferior surface 706 may be configured to replicate an exemplary natural state for the prosthesis 102, where the upper plate 200 is offset slightly forward of, and tilted slightly toward, the lower plate 202. For example, the superior surface 704 may be offset from the inferior surface 706 by approximately 0.5 mm. In addition, the superior surface 704 may be angled toward the inferior surface 706 by approximately 3°. It is noted, however, that this is only an exemplary state, and that other natural states are contemplated, without departing from the scope of the present disclosure.

In embodiments, the body 702 of the trial implant 600 includes a crossmember 728. In some embodiments, the crossmember 728 is a thickness that is less than a total thickness of the trial implant 600. This allows the body 702 of the trial implant 600 to be inserted into the opening 130 at the proximal end 116 of the instrument 106 when the locking shaft 402 is in a first orientation and may be secured within the opening 130 when the locking shaft 402 is in a second orientation. Thus, as illustrated in FIGS. 7A-7G, in some embodiments the upper surface 614 and the lower surface 618 of the trial implant 600 are substantially flat or planar along the entire length of the body 702, aside from the wedges 612, 616 that extend from the respective surfaces 614, 618.

The body 702 additionally includes a first tail 730 and a second tail 732 at a distal end 606 of the trial implant 600. The space between the first tail 730 and the second tail 732 defines a slot 734 of the distal end 606 of the trial implant 600. In addition, the crossmember 728 and the tails 730, 732 define a cavity 736 of the distal end 606 of the trial implant 600.

In some embodiments, the cavity 736 has a greater width than the slot 734. As with the cartridge 104, the coupler 426 of the locking shaft 402 of the instrument 106 may be inserted within the slot 734 when in a first orientation and re-oriented within the cavity 736 to a second orientation, causing the coupler 426 of the instrument 106 to be retained within the cavity 736 by the slot 734 when the coupler 426 is in the second orientation.

In embodiments, the body 702 optionally includes a cavity 738 between the head 700 and the crossmember 728. For example, the cavity 738 may be beneficial for weight reduction of the trial implant 600, so as to more closely emulate an assembly 534 of a cartridge 104 and a particular prosthesis 102.

In embodiments, a surface of the trial implant 600 includes a plurality of indicators 740. The plurality of indicators 740 may be marks (e.g., laser-etched, drawn, an applied sticker, or the like), colors, and/or patterns that are on the surface. For example, the surface may be the superior surface 704 of the head 700, so that the plurality of indicators 740 are visible to the user when the trial implant 600 is inserted in the instrument 106. In general, the plurality of indicators 740 may include information about the trial implant 600, to assist in the selection of the corresponding prosthesis 102 (and/or corresponding cartridge and prosthesis assembly 534). In some instances, the plurality of indicators 740 may include, but are not limited to, information about the depth (Dxx), width (Wxx), and/or height (Hxx) of the head 700.

It is contemplated that the body 702 may include an opening between the cavities 736 and 738. For example, the opening may allow for passage of the locking pin 444, including where the locking pin 444 is installed in the instrument 106 and/or is integrated with the release driver 404, as opposed to being provided with the cartridge and prosthesis assembly 534.

FIG. 8 in general illustrates the osteotome 602 of the system 100, in accordance with one or more embodiments of the present disclosure. It should be understood that any embodiment of the osteotome 602 illustrated in FIGS. 6A-6G may be directed to the osteotome 602 illustrated in FIG. 8, and vice versa, without departing from the scope of the present disclosure.

In embodiments, the osteotome 602 includes a body 800 with a tip 802 at the proximal end 608. The tip 802 is dimensioned to fit within the channel 122 defined by the at least one depth stop 120 on the instrument 106. Optionally, the tip 802 may be dimensioned to fit within the alignment feature 420 of the instrument 106 (e.g., so that the alignment feature 420 assists in positioning of the tip 802, such as within the patient at the implantation site).

In some embodiments, the tip 802 is used to mark a midline on the vertebral body using a tip 802 at the proximal end 608 of the osteotome 602. A user may apply a force (e.g., either a constant applied force or a striking force) on the body 800 at the distal end 610 of the osteotome 602 to cause the tip 802 to make the midline mark on the vertebral body.

Although embodiments of the present disclosure are directed to the use of the osteotome 602 to mark the midline on a vertebral body, it should be understood that alternate tools may be used to instead mark the midline. For example, a bovie or electric scalpel may be used to mark the midline, without departing from the scope of the present disclosure. It is noted that the alternate tools may be used with the instrument 106 (e.g., including by being insertable with the channel 122 between the depth stops 120), or may be used separately from the instrument 106.

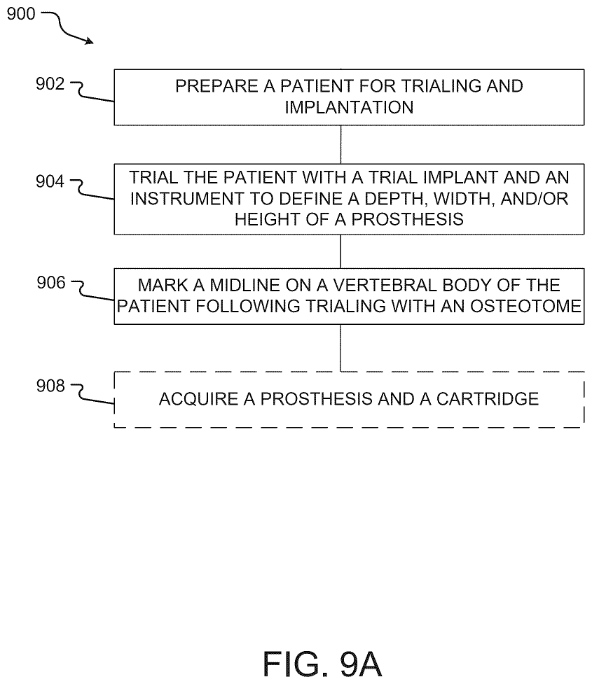

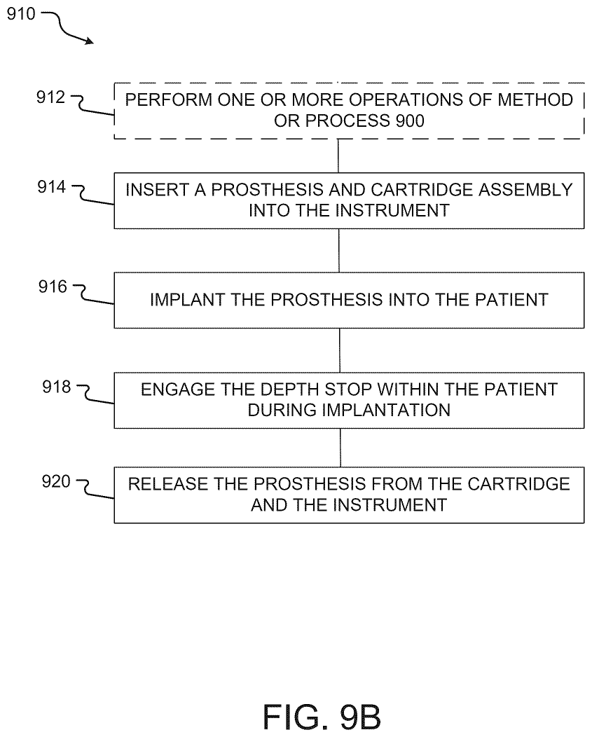

FIGS. 9A and 9B are method or process 900, 910 flow diagrams, respectively illustrating the operation of the intervertebral disc prosthesis system 100, in accordance with one or more embodiments or the present disclosure. While a general order for the steps of the method or process is shown in FIGS. 9A and 9B, the method or process can include more or fewer steps or can arrange the order of the steps differently (including simultaneously, substantially simultaneously, or sequentially) than those shown in FIGS. 9A and 9B. It is noted that the method or process shall be explained with reference to the components, devices, subassemblies, environments, etc. described in conjunction with FIGS. 1A-8. For example, it is noted that the embodiments as illustrated in FIGS. 1A-8 should be understood as being directed to the embodiments described with respect to FIGS. 9A and 9B, and vice versa, without departing from the scope of the present disclosure.

Referring now to FIG. 9A, in embodiments, a patient is prepared 902 for trialing and implantation. For example, a discectomy may be performed on the patient to create a cavity between adjacent vertebral bodies, to be trialed with a trial implant 600 and subsequently receipt of a particular prosthesis 102.

In embodiments, the patient is trialed 904 with a trial implant and an instrument to define a depth, width, and/or height of a prosthesis. A trial implant 600 is inserted into an opening 130 of an instrument 106, and a coupler 426 of the instrument 106 engages the trial implant 600. The trial implant 600 is inserted into the dissected cavity between the adjacent vertebral bodies. Trialing the patient may include one or more of centering an alignment feature 722 of the head 700 of the trial implant 600 within the cavity, and/or using an actuator 414 of the instrument 106 to adjust a position of at least one depth stop 120 on the outer housing 400 of the instrument 106 relative to the trial implant 600, thereby adjusting a depth that a prosthesis 102 will be inserted into the defined cavity between adjacent vertebral bodies.

Optionally, the trial implant 600 may be removed from the defined cavity, interchanged with a different trial implant 600 on the instrument 106, and re-inserted into the defined cavity. This can be repeated until the appropriate depth, width, and/or height for a particular prosthesis 102 is determined, and may require multiple trialing attempts.

In embodiments, a midline is marked 906 on a vertebral body following the trialing with an osteotome (or other tool such as a bovie or electric scalpel, or the like). The midline is marked with an osteotome 602 inserted into a channel 122 defined by the at least one depth stop 120. For example, the midline may be marked on an anterior wall of an upper vertebral body of the adjacent vertebral body, at a location proximate to the channel 122 and the at least one depth stop 120 on the instrument 106.

In optional embodiments, a prosthesis and a cartridge are acquired 908. Based on the determination of the appropriate prosthesis 102 with the trial implant 600, a cartridge 104 and the prosthesis 102 may be acquired. The prosthesis 102 is determined based on the defined depth, width, and/or height from trialing. The cartridge 104 and the prosthesis 102 may be components of a cartridge and prosthesis assembly 534. Optionally, the cartridge and prosthesis assembly 534 may include the locking pin 444 with the expansion bit 438 as part of the assembly.

It is noted that the cartridge 104, the prosthesis 102, and optionally the locking pin 444 with the expansion bit 438 may be acquired in a pre-assembled state or may be acquired as a kit that is assembled by the user prior to implantation. For example, where the prosthesis 102 and the cartridge 104 are assembled, the prosthesis 102 may be coupled to the cartridge 104 via the shelves 326, 330, 334, 338 and the ledges 240, 260, and the tabs 328, 332, 336, 340 and the notches 242, 262 to form an assembly 534. Optionally, the locking pin 444 including the expansion bit 438 of a release driver 404 of the instrument 106 is assembled with the cartridge 104 and the prosthesis 102 in the assembly 534.

In general, it should be understood that the prosthesis 102, the cartridge 104, and/or the locking pin 444 may each be acquired as separate fully assembled components or may be assembled by a user during one or more steps of the process 900, without departing from the scope of the present disclosure.

Referring now to FIG. 9B, in optional embodiments one or more operations of the method or process 900 are performed 912. It is contemplated that the same individual may perform one or more operations of the methods or processes 900, 910. Alternatively, it is contemplated that a first subset of operations of the methods or processes 900, 910 may be performed by a first individual, and at least a second subset of operations of the methods or processes 900, 910 may be performed by at least a second individual, without departing from the scope of the present disclosure.

In embodiments, an assembly including the prosthesis and the cartridge is inserted 914 into the instrument. Where the trial implant 600 has not already been removed, the trial implant 600 is first removed from the opening 130 of the instrument 106. The assembly 534 including the prosthesis 102 and the cartridge 104 (and, optionally, the locking pin 444 including the expansion bit 438 of the release driver 404) is then inserted into the opening 130 of the instrument 106.

It is noted that insertion of the assembly 534 into the opening 130 of the instrument 106 should be accomplished without moving the depth stop adjustment, as the particular prosthesis 102 may be selected based on the setting of the at least one depth stop 120.

In embodiments, the prosthesis is implanted 916 into the patient. Using the instrument 106, the prosthesis 102 is inserted into the cavity between the adjacent vertebral bodies that was previously trialed. During insertion, alignment indicators (e.g., the set of alignment teeth 246 and/or the set of alignment teeth 266 on the prosthesis 102, and/or the alignment feature 420 on the instrument 106) are aligned with the mark made with the osteotome 602, to conform to the midline defined during trialing.

In embodiments, the depth stop is engaged 918 within the patient during implantation. The instrument 106 is inserted into the patient, and thus the prosthesis 102 is implanted within the cavity defined between the adjacent vertebral bodies, until the at least one depth stop 120 makes contact with the upper vertebral body of the adjacent vertebral bodies.

In embodiments, the prosthesis is released 920. Following contact of the at least one depth stop 120 with the upper vertebral body, the cartridge 104 is disengaged from the prosthesis 102 to allow for removal of the instrument 106 with the cartridge 104 from the patient, while the prosthesis 102 remains implanted. For example, the release driver 404 is actuated via an actuator 418, such that the expansion bit 438 engages release tabs 360, 362 of the cartridge 104, to cause the arms 300, 302 of the cartridge 104 to expand and release the prosthesis 102. It is noted that, where the release driver 404 includes the actuator portion 442 and the locking pin 444, the actuator portion 442 including the actuator 418 may be inserted into the instrument 106 and engage the locking pin 444 (e.g., via the interlocking assembly 440) prior to the actuation of the release driver 404.

In embodiments, one or more components of the system 100 may be agnostic to the patient into which the prosthesis 102 is to be installed. For example, the prosthesis 102, the cartridge 104, the instrument 106, the trial implant 600, and/or the osteotome 602 may be agnostic to the patient, and able to interface with one another regardless of the size of the prosthesis 102 and the cartridge 104/the cartridge 104 or the trial implant 600 and the instrument 106.

In embodiments, the prosthesis 102 and/or the trial implant 600 may be configured to be usable for a percentile range of patients (e.g., a 5% to 95% percentile range).

In embodiments, one or more components of the system 100 may be patient-specific. For example, the prosthesis 102 may have a customized configuration designed using pre-operative planning procedures (e.g., including, but not limited to, acquiring scan data, modelling the prosthesis, fabricating the prosthesis based on the modelling, modifying a pre-fabricated prosthesis based on the acquired scan data) based on a particular patient, using modelling technologies on a computer with processors and memory able to store and execute instructions for the processor. As such, it should be understood that at least a portion of the present disclosure may be directed to a computer-implemented method and/or a computer-implemented invention. In addition, it should be understood that the method or process 900 may include one or more operations, either in the same flow or in an accompanying flow, for the pre-operative planning procedures to produce the patient-specific prosthesis 102.

It should be understood that embodiments directed to the system 100 and the methods or processes 900, 910 are similarly applicable to a system 1200 (including for use with or in the methods or processes 900, 910), the system 1200 being described in detail further herein.

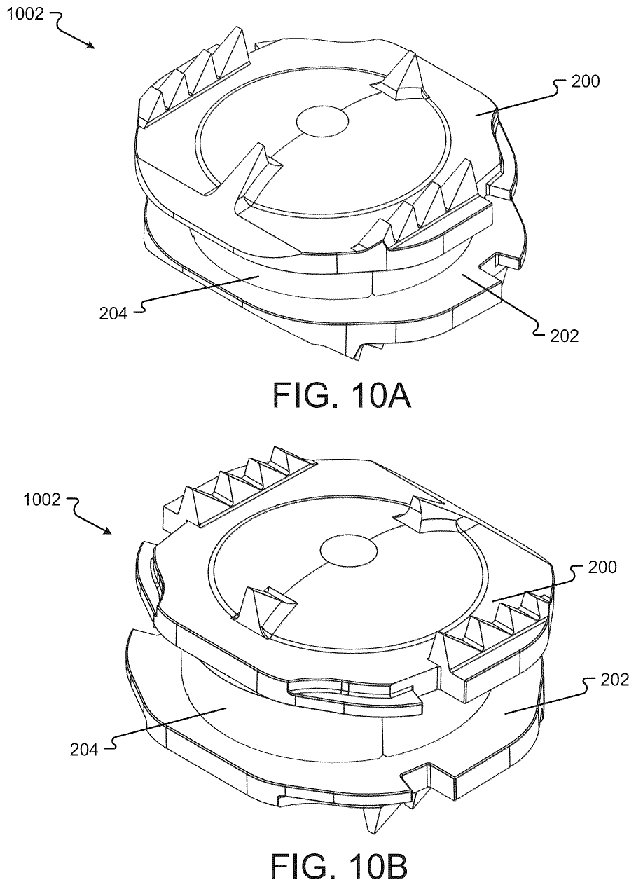

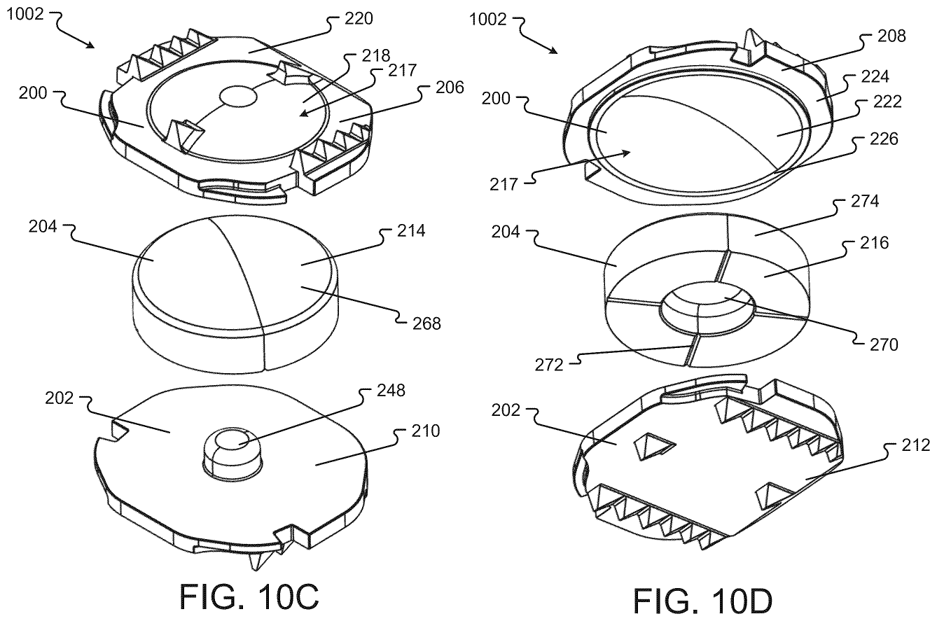

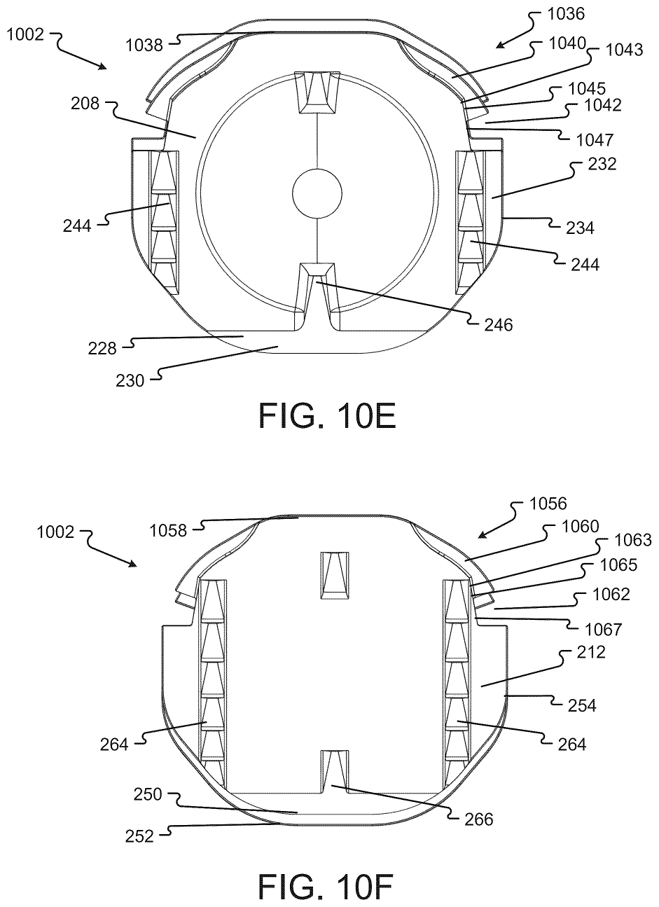

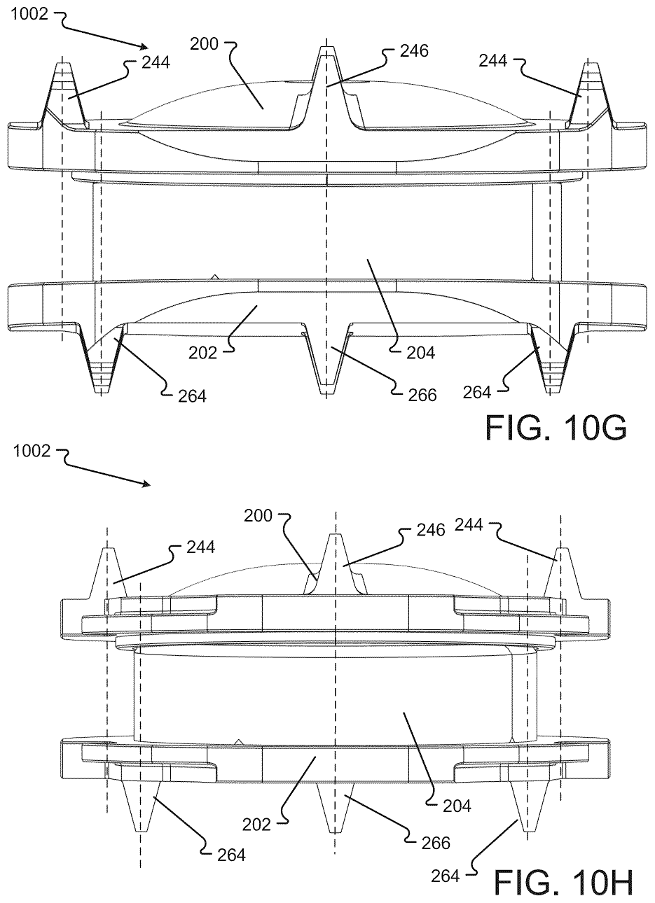

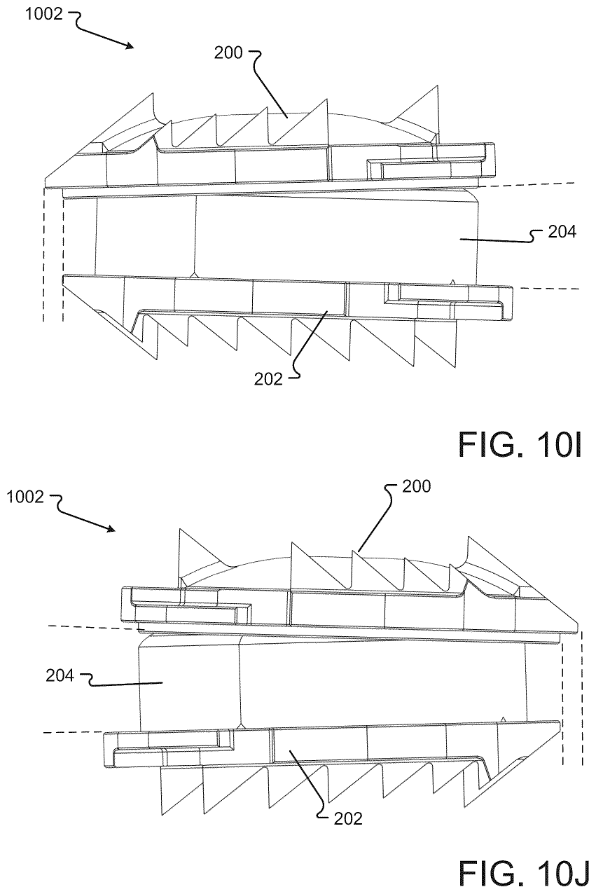

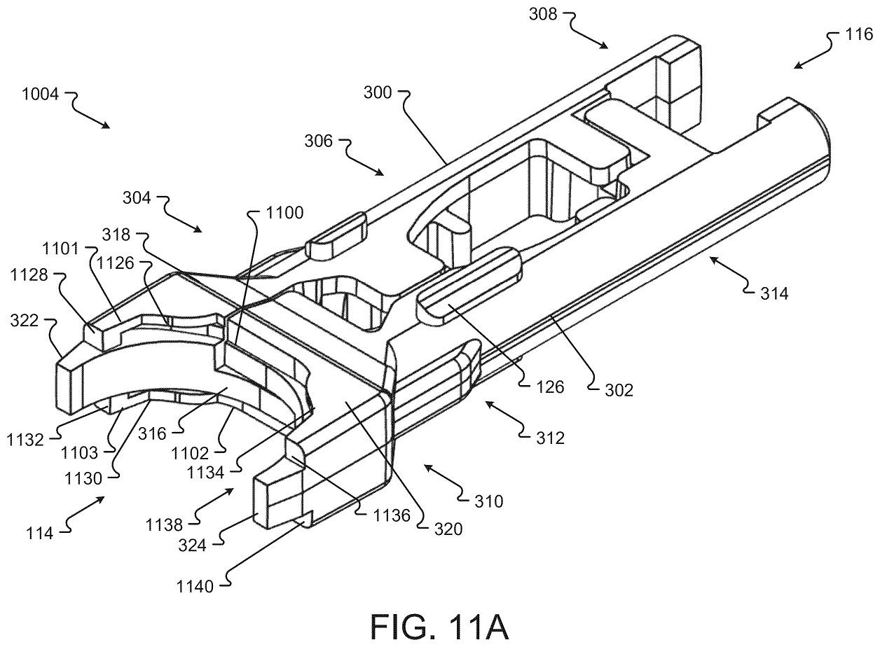

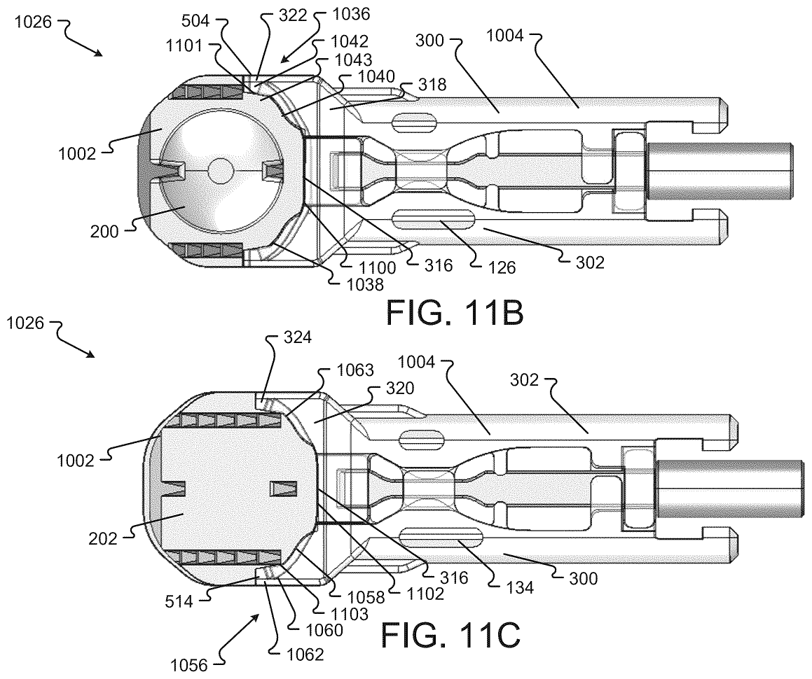

FIGS. 10A-10J and 11A-11C in general illustrate a variation of the prosthesis 102 of the system 100, labeled a prosthesis 1002, in accordance with one or more embodiments of the present disclosure. It should be understood that any embodiment of the prosthesis 1002 illustrated in FIGS. 10A-10J and 11A-11C may be directed to the prosthesis 102 illustrated in FIGS. 1A-1G, FIGS. 2A-2L, and/or FIGS. 5A-5H, and vice versa, without departing from the scope of the present disclosure unless otherwise noted.

In addition, FIGS. 11A-11C in general illustrate a variation of the cartridge 104 of the system 100, labeled a cartridge 1004, in accordance with one or more embodiments of the present disclosure. Although not shown in FIGS. 11A-11C, it should be understood that the cartridge 1004 is usable with the instrument 106 of the system 100 as illustrated throughout the present disclosure. In addition, it should be understood that any embodiment of the cartridge 1004 illustrated in FIGS. 11A-11C may be directed to the cartridge 104 illustrated in FIGS. 1A-1G, FIGS. 3A-3H, and/or FIGS. 5A-5H, and vice versa, without departing from the scope of the present disclosure.

It should be understood that the prosthesis 1002 and the cartridge 1004 are usable with the process 900 in place of the prosthesis 102 and the cartridge 104, without departing from the scope of the present disclosure. The prosthesis 1002 and the cartridge 1004 together form a prosthesis and cartridge assembly 1026 able to engage with the instrument 106 as described throughout the present disclosure. In embodiments, the prosthesis 1002 and the cartridge 1004 may be understood as differing from the prosthesis 102 and the cartridge 104, respectively, with respect to the engagement between the prosthesis 1002 and the cartridge 1004.

In embodiments, as illustrated in FIG. 10E, the cartridge 1002 has cartridge engagement features 1036 on a rear edge 1038 of the upper plate 200 with a ledge 1040 and a notch 1042. Unlike the cartridge 104 as illustrated in at least FIG. 2E, in which a portion 243 of the superior surface 206 is defined by the ledge 240 and the notch 242, a portion 1043 of the superior surface 206 is only defined by the ledge 1040. Notably, the notch 1042 extends a depth into the superior surface 206 no further than the ledge 1040, such that a rear edge or surface 1045 of the ledge 1040 also forms a rear edge or surface 1047 of the notch 1042.

As illustrated in FIGS. 11A and 11B, the first arm 300 and the second arm 302 of the cartridge 1004 define a first edge 1100 of the receiver 316 of the cartridge 1004, where the first edge 1100 has one or more substantially straight portions 1101 at the proximal end of a respective hand 318 or hand 320 of the cartridge 1004, as compared to the one or more portions 501 of the first edge 500 of the cartridge 104 (e.g., as illustrated in at least FIGS. 5B-5D). The portions 1101 of the first edge 1100 of the receiver 316 that are contoured to conform to the rear edge 1038 of the upper plate 200, and specifically that are contoured to conform to the engagement portions 1043 along the rear edge 1038, may reduce manufacturing costs, reduce the possibility of failure to disengage, and/or reduce the possibility of unseating the prosthesis 1002 by the cartridge 1004 after insertion into a patient at an implantation site.

It is noted that the first hand 318 optionally includes shelves 1126 and 1130 that are positioned relative to the ledges 1040 and 1060 on the upper plate 200 and lower plate 202 of the prosthesis 1002, respectively, to assist in securing the prosthesis 1002 within the cartridge 1004. In addition, it is noted that the first hand 318 optionally includes tabs 1128 and 1132 that engage notches 1042 and 1062 on the upper plate 200 and lower plate 202 of the prosthesis 1002 to assist in securing the prosthesis 1002 within the cartridge 1004, respectively. A difference between the assembly 1026 versus the assembly 534, however, is the removal of the portion 243 of the prosthesis 102 and portion 501 of the cartridge 104, and the inclusion of the respective portions 1101, 1103 of the cartridge 1004 that engages with the rear edges 1038, 1058 of the plates 200, 202 of the prosthesis 1002.

In embodiments, as illustrated in FIG. 10F, the cartridge 1002 has cartridge engagement features 1056 on a rear edge 1058 of the lower plate 202 with a ledge 1060 and a notch 1062. Unlike the cartridge 104 as illustrated in FIG. 2F, in which a portion 263 of the inferior surface 212 is defined by the ledge 260 and the notch 262, a portion 1063 of the inferior surface 212 is only defined by the ledge 1060. Notably, the notch 1062 extends a depth into the inferior surface 212 no further than the ledge 1060, such that a rear edge or surface 1065 of the ledge 1060 also forms a rear edge or surface 1067 of the notch 1062.

As illustrated in FIGS. 11A and 11C, the first arm 300 and the second arm 302 of the cartridge 1004 define a second edge 1102 of the receiver 316 of the cartridge 1004, where the second edge 1102 has one or more substantially straight portions 1103 at the proximal end of a respective hand 318 or hand 320 of the cartridge 1004, as compared to the one or more portions 503 of the second edge 502 of the cartridge 104 (e.g., as illustrated in at least FIGS. 5B-5D). The portions 1103 of the second edge 1102 of the receiver 316 that are contoured to conform to the rear edge 1058 of the lower plate 202, and specifically that are contoured to conform to the engagement portions 1063 along the rear edge 1058, may reduce manufacturing costs, reduce the possibility of failure to disengage, and/or reduce the possibility of unseating the prosthesis 1002 by the cartridge 1004 after insertion into a patient at an implantation site.

It is noted that the second hand 320 optionally includes shelves 1134 and 1138 that are positioned relative to the ledges 1040 and 1060 on the upper plate 200 and lower plate 202 of the prosthesis 1002, respectively, to assist in securing the prosthesis 1002 within the cartridge 1004. In addition, it is noted that the second hand 320 optionally includes tabs 1136 and 1140 that engage notches 1042 and 1062 on the upper plate 200 and lower plate 202 of the prosthesis 1002, respectively, to assist in securing the prosthesis 1002 within the cartridge 1004. A difference between the assembly 1026 versus the assembly 534, however, is the removal of the portion 243 of the prosthesis 102 and portion 501 of the cartridge 104, and the inclusion of the respective portions 1101, 1103 of the cartridge 1004 that engages with the rear edges 1038, 1058 of the plates 200, 202 of the prosthesis 1002.

It is noted that the arms 300, 302 of the cartridge 1004 are shown as transparent structures to better illustrate the interface between the cartridge engagement features 1036, 1056 of the prosthesis 1002 and the receiver 316 of the cartridge 1004.

Although the interface between the prosthesis 1002 and the cartridge 1004 may be different due to the differences described above with respect to FIGS. 10A-10J and 11A-11C, it is noted that the prosthesis 1002 and the cartridge 1004 otherwise maintains relationships between corresponding surfaces (e.g., the surfaces being substantially flush or planar) as described with respect to the prosthesis 102 and the cartridge 104, without departing from the scope of the present disclosure. In addition, it is noted that the cartridge 1004 maintains relationships with the instrument 106 (e.g., being dimensioned to fit within the opening 130 of the instrument 106, inclusion of the wedges 126, 134 on the cartridge 1004 that engage with the respective surfaces 128, 136 of the instrument 106, the like), without departing from the scope of the present disclosure.

In embodiments, as illustrated in at least FIGS. 3A-3H, the upper wedges 126 may be substantially equal in length, and/or the lower wedges 134 may be substantially equal in length, between the ends 116, 118 of the instrument 106. In other embodiments, as illustrated in FIGS. 11A-11C, an upper wedge 126 may be a different length (e.g., a longer length, or a shorter length) than another upper wedge 126, and/or a lower wedge 134 may be a different length (e.g., a longer length, or a shorter length) than another lower wedge 134, between the ends 116, 118 of the instrument 106.

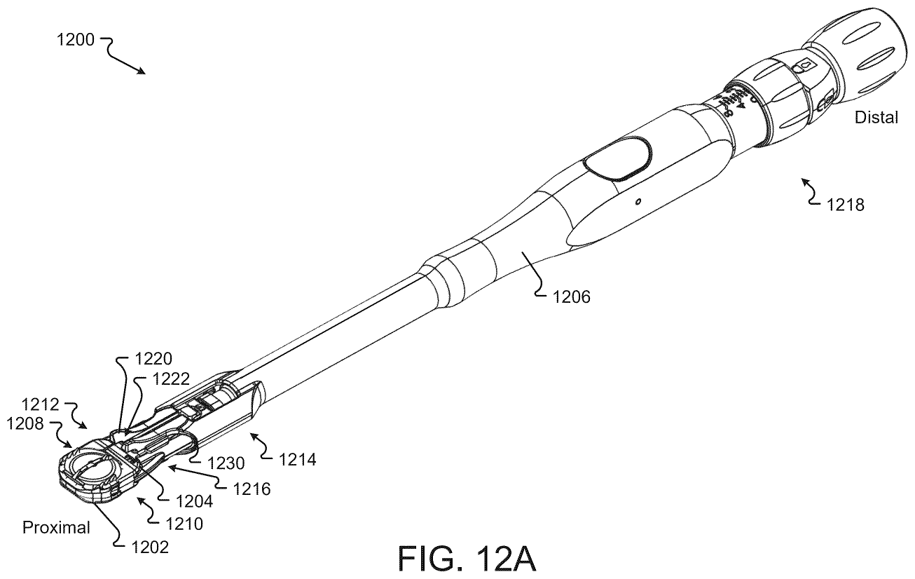

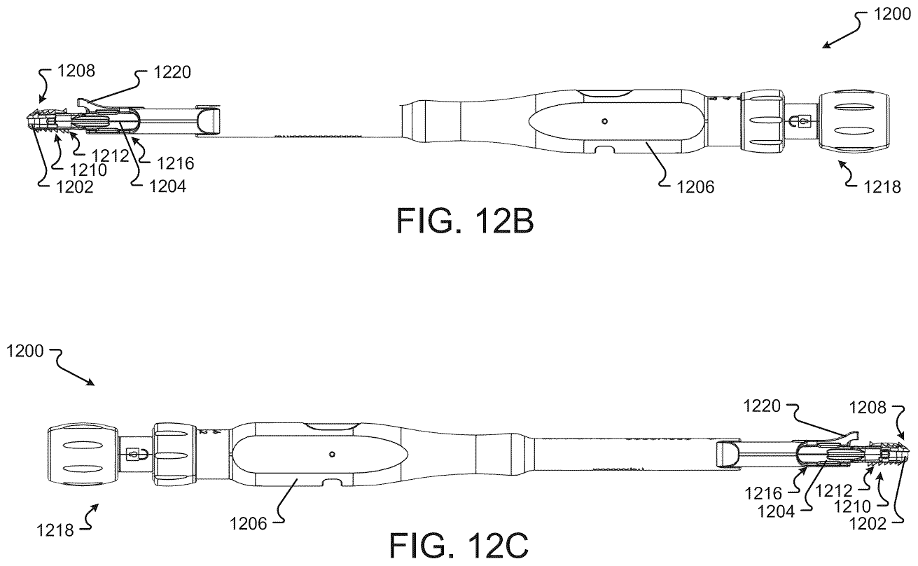

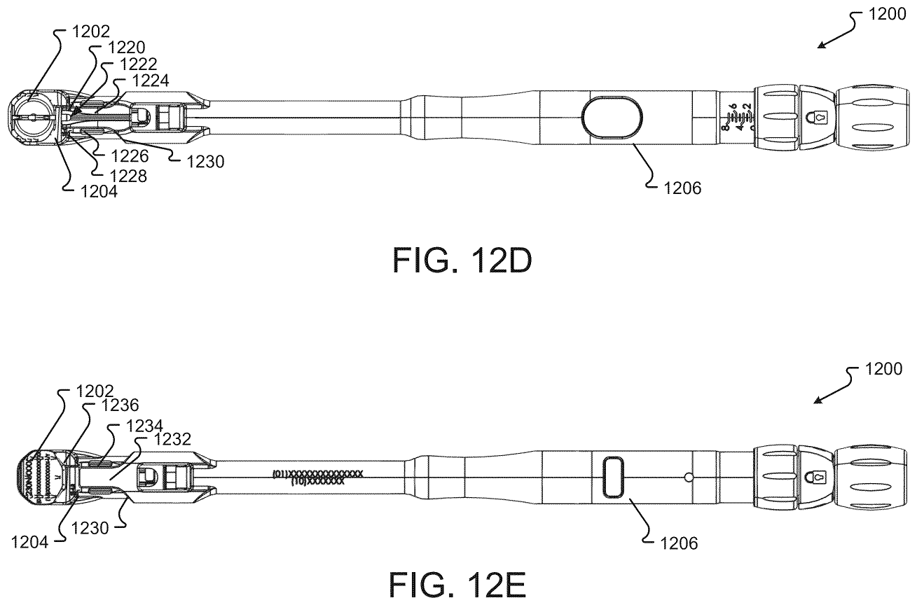

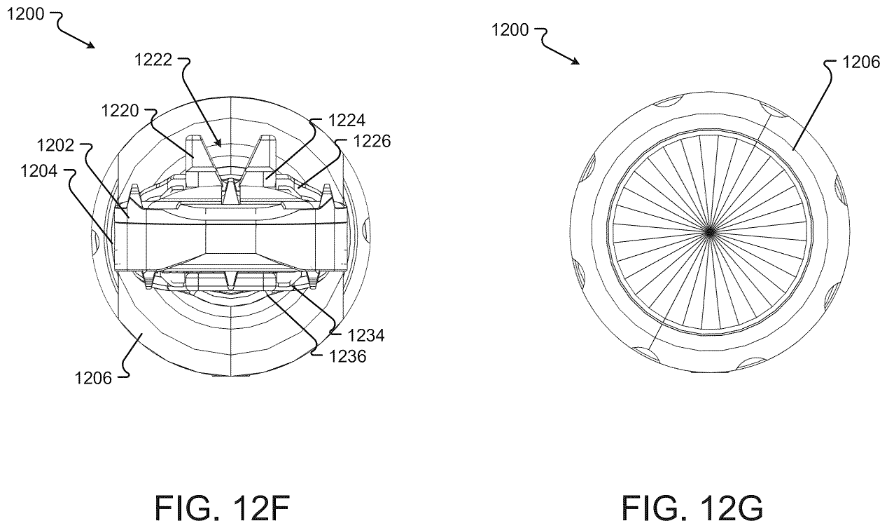

FIGS. 12A-12G in general illustrate an intervertebral disc prosthesis system 1200, in accordance with one or more embodiments of the present disclosure. The system 1200 includes one or more of a prosthesis 1202, a cartridge 1204, and an instrument 1206. Reference will be made to each of the subassemblies 1202, 1204, 1206 in detail further herein. In addition, any and/or all of the subassemblies 1202, 1204, 1206 may include further subassemblies as described in detail further herein, which are each considered subassemblies of the system 1200.

It should be understood that one or more components 1202, 1204, 1206 of the system 1200 are interchangeable with the components 102, 104, 106, and/or with the components 1002, 1004, of the system 100. In addition, it should be understood that the components 1202, 1204, 1206 of the system 1200 are usable with the method or process 900 in place of and/or with the components 102, 104, 106 of the system 100, without departing from the scope of the present disclosure.

As illustrated in FIGS. 12A-12G, the prosthesis 1202 has a proximal end 1208 and a distal end 1210. The cartridge 1204 has a proximal end 1212 and a distal end 1214. The instrument 1202 has a proximal end 1216 and a distal end 1218.

The proximal end 1212 of the cartridge 1204 receives the distal end 1210 of the prosthesis 1202. The distal end 1214 of the cartridge 1204 is insertable into the proximal end 1216 of the instrument 1206. In some embodiments, the proximal end 1212 of the cartridge 1204 receives the distal end 1210 of the prosthesis 1202 prior to insertion of the distal end 1214 of the cartridge 1204 into the proximal end 1216 of the instrument 1206. In other embodiments, the proximal end 1212 of the cartridge 1204 receives the distal end 1210 of the prosthesis 1202 after insertion of the distal end 1214 of the cartridge 1204 into the proximal end 1216 of the instrument 1206.

In some embodiments, the proximal end 1216 of the instrument 1206 includes at least one depth stop 1220 that defines a channel 1222 on an upper tab 1224. In some non-limiting examples, the channel 1222 is defined by two depth stops 1220.

The upper tab 1224 of the instrument 1206 is configured to engage at least one upper wedge 1226 on an upper surface 1228 of the cartridge 1204, to assist in aligning the cartridge 1204 within an opening 1230 in the proximal end 1216 of the instrument 1206. For example, the upper surface 1228 may include two upper wedges 1226 that flank the upper tab 1224 of the instrument 1206 during insertion of the cartridge 1204 into the opening 1230 of the instrument 1206.

In other embodiments, a lower tab 1232 of the instrument 1206 (e.g., at the proximal end 1216 of the instrument 1206) is configured to engage at least one lower wedge 1234 on a lower surface 1236 of the cartridge 1204, to assist in aligning the cartridge 1204 within the opening 1230 in the proximal end 1216 of the instrument 1206. For example, the lower surface 1236 may include two lower wedges 1234 that flank the lower tab 1232 of the instrument 1206 during insertion of the cartridge 1204 into the opening 1230 of the instrument 1206.

It is noted that the cartridge 1204 may include the upper wedges 1226 and/or the lower wedges 1234 either individually or in combination, without departing from the scope of the present disclosure.

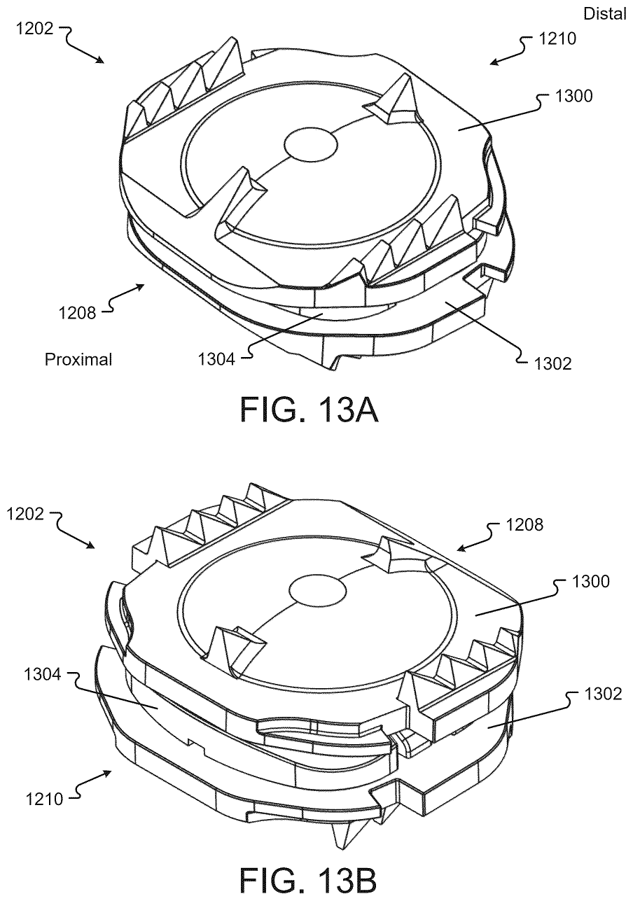

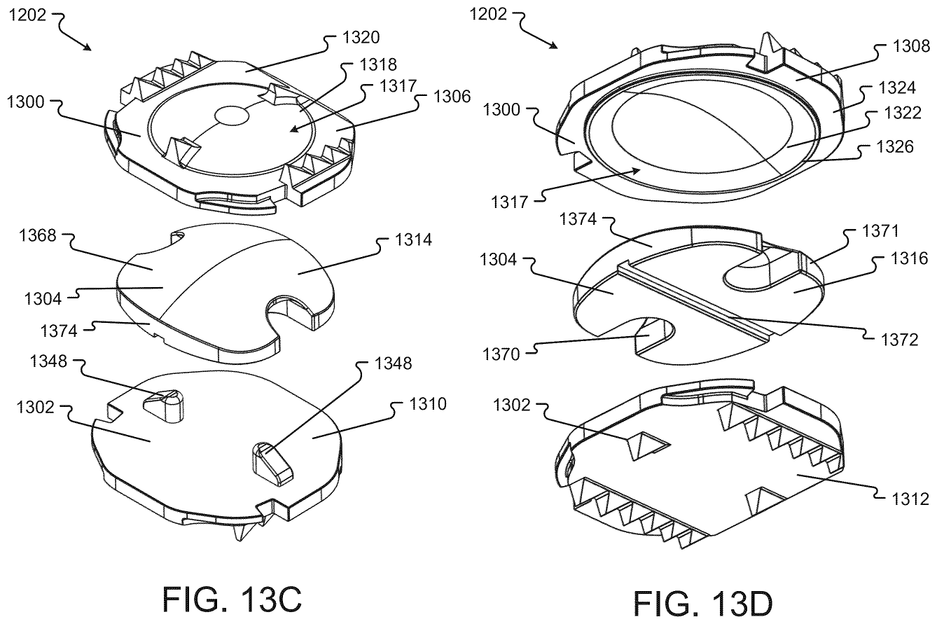

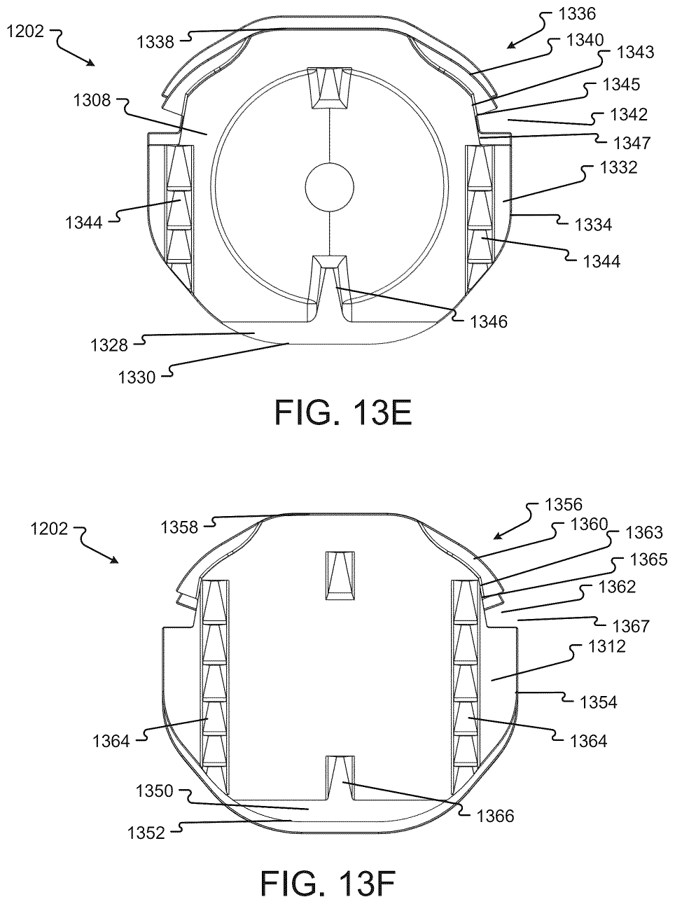

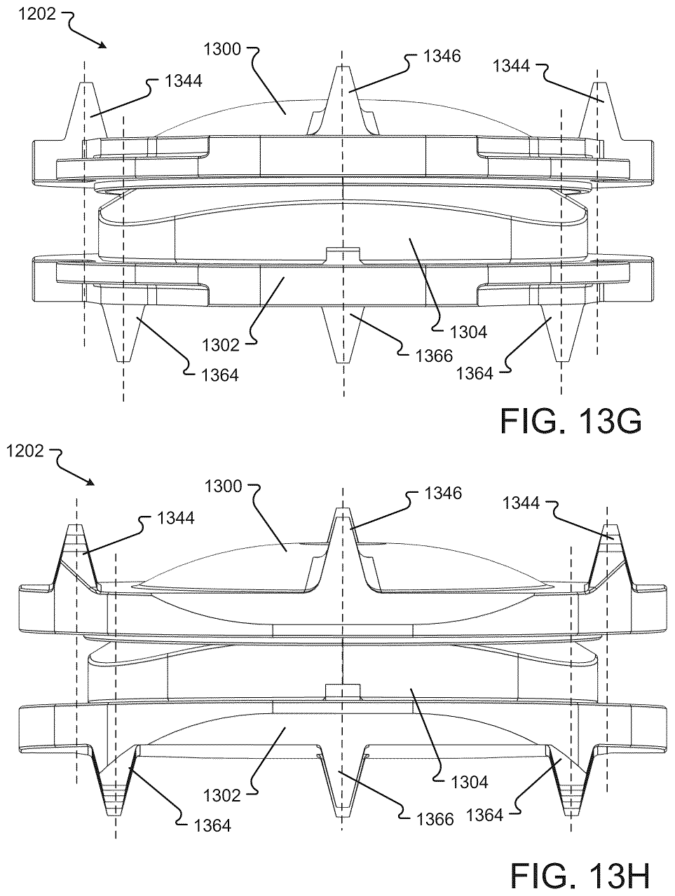

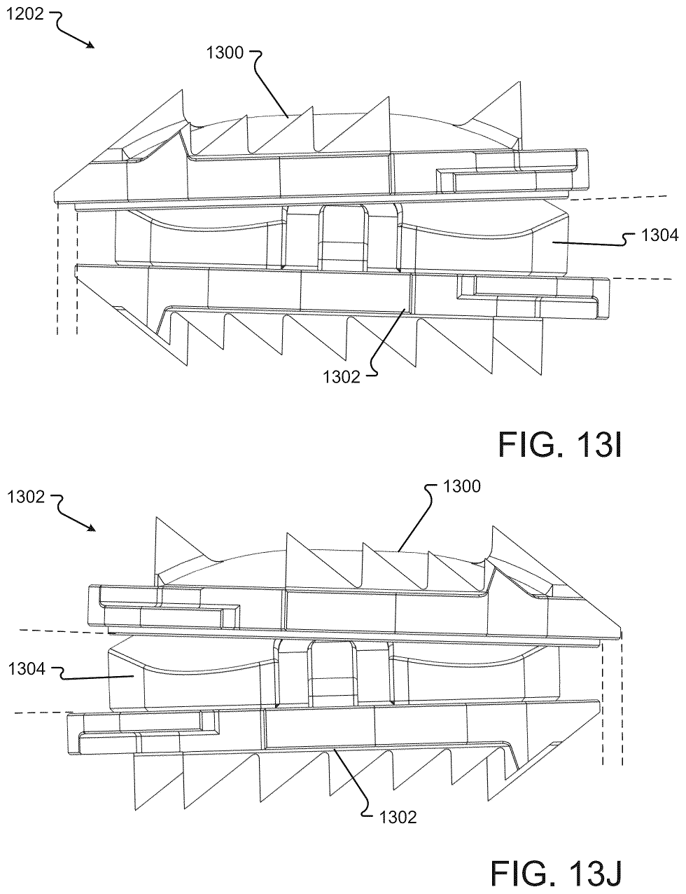

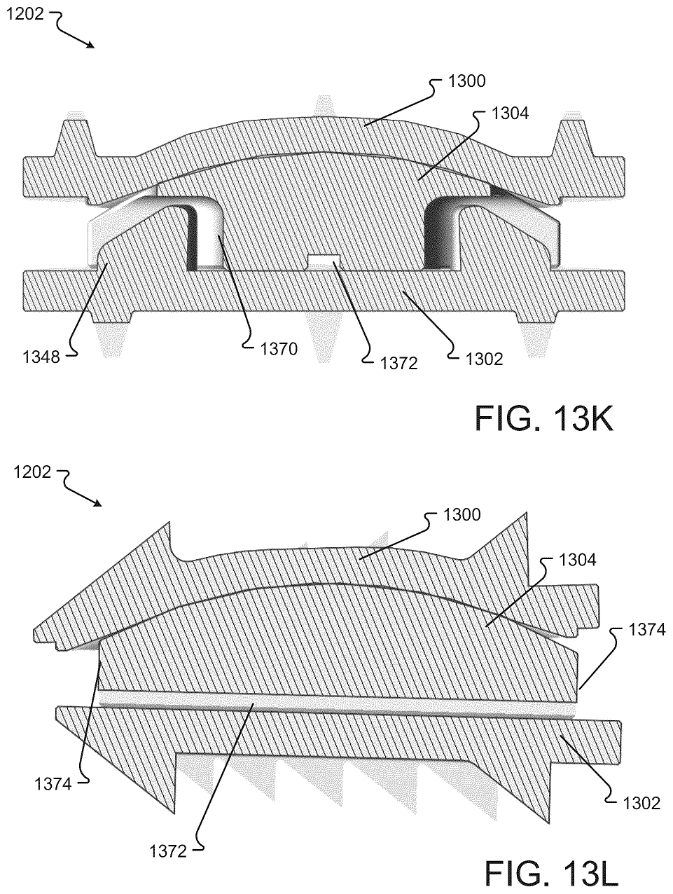

FIGS. 13A-13L in general illustrate the prosthesis 1202 of the system 1200, in accordance with one or more embodiments of the present disclosure. It should be understood that any embodiment of the prosthesis 1202 illustrated in FIGS. 12A-12G may be directed to the prosthesis 1202 illustrated in FIGS. 13A-13L, and vice versa, without departing from the scope of the present disclosure.

In embodiments, the prosthesis 1202 includes an upper plate (or superior plate) 1300, a lower plate (or inferior plate) 1302, and an insert (or central core) 1304. The upper plate 1300 has a superior surface 1306 and an inferior surface 1308. The lower plate 1302 has a superior surface 1310 and an inferior surface 1312. The insert 1304 has a superior surface 1314 and an inferior surface 1316.

In some embodiments, at least one of the upper plate 1300 and/or the lower plate 1302 is fabricated from a metal or metal alloy. For example, the metal or metal alloy may include, but is not limited to, a Titanium (Ti) alloy. For instance, the metal or metal alloy may include an alloy comprising Titanium (Ti), Aluminum (Al), and Vanadium (V) (e.g., Ti₆Al₄V).

In some embodiments, at least one of the superior surface 1306 of the upper plate 1300 and/or the inferior surface 1312 of the lower plate 1302 is coated. For example, the coating may include, but is not limited to, a bioceramic. For instance, the bioceramic may include hydroxyapatite (Ca₁₀(PO₄)₆(OH)₂) (HAP), to create a Ti/HAP coating on the superior surface 1306 of the upper plate 1300 and/or the inferior surface 1312 of the lower plate 1302. In some instances, a bioceramic coating such as Ti/HAP may improve osseointegration between the prosthesis 1202 and respective contact surfaces of the adjacent vertebral bodies between which the prosthesis 1202 is implanted.

In some embodiments, the insert 1304 is fabricated from a polymer. For example, the polymer may include, but is not limited to, a polyethylene (PE)-based polymer. For instance, the PE-based polymer may be infused with Vitamin E, to create a PE Vit E polymer.

In some embodiments, at least one of the inferior surface 1308 of the upper plate 1300 and/or the superior surface 1310 of the lower plate 1302 is coated. For example, the coating may include, but is not limited to, a friction enhancement coating. For instance, the friction enhancement coating reduces the friction coefficient and beneficially limits wear debris from forming within the implantation site. In addition, the friction enhancement coating reduces the possibility that the insert 1304 exits the cavity defined between the upper plate 1300 and the lower plate 1302.

In embodiments, the upper plate 1300 includes a dome region 1317. In general, the dome region is contoured to conform to the superior surface 1314 of the insert 1304, which is optionally convex and dome-shaped. For instance, the superior surface 1314 of the insert 1304 may be contoured to conform to a substantially spherical portion for the convex dome shape.

For example, the dome region 1317 may include a convex portion 1318 at least partially surrounded by a substantially flat or planar portion 1320 in the superior surface 1306. The transition between the convex portion 1318 and the substantially flat or planar portion 1320 may be sloped and/or without a step-like change in height. Alternatively, the transition between the convex portion 1318 and the substantially flat or planar portion 1320 may have a step-like change in height.

By way of another example, the dome region 1317 may include a concave portion 1322 at least partially surrounded by a substantially flat or planar portion 1324 in the inferior surface 1308. The transition between the concave portion 1322 and the substantially flat or planar portion 1324 may have a step-like change in height. For example, the step-like change in height may be formed by a substantially flat or rounded rim 1326 that extends away from (e.g., downward) from the inferior surface 1308. The transition between the convex portion 1318 and the substantially flat or planar portion 1320 may be sloped and/or without a step-like change in height.

It should be understood that the upper plate 1300 may include both portions 1318, 1322, or only one of the portions 1318, 1322, without departing from the scope of the present disclosure. For example, the upper plate 1300 may include the concave portion 1322 in the inferior surface 1308, but a substantially flat or planar superior surface 1306 without the convex portion 1318.

In embodiments, the upper plate 1300 includes a chamfer 1328 on a front edge or leading edge 1330. For example, the chamfer 1328 may facilitate insertion of the prosthesis 1202 into the cavity defined between the adjacent vertebral bodies during implantation. Optionally, the upper plate 1300 includes a substantially flat or planar surface 1332 on one or more side edges 1334. For example, both side edges 1334 may include the substantially flat or planar surface 1332. It is noted that the substantially flat or planar surface 1332 may facilitate insertion of the prosthesis 1202 into the cavity defined between the adjacent vertebral bodies during implantation.

In embodiments, the upper plate 1300 includes at least one cartridge engagement feature 1336 on a rear edge or trailing edge 1338. For example, the upper plate 1300 includes two opposing cartridge engagement features 1336 on the rear edge 1338. The at least one cartridge engagement feature 1336 includes a ledge 1340 within the superior surface 1306, and a notch 1342 through the thickness of the upper plate 1300 and between the superior surface 1306 and the inferior surface 1308. In this regard, the at least one cartridge engagement feature 1336 may be considered to have a tiered structure or tiered configuration between the inferior surface 1308, a surface of the ledge 1340, and the superior surface 1306 of the upper plate 1300, for purposes of the present disclosure.

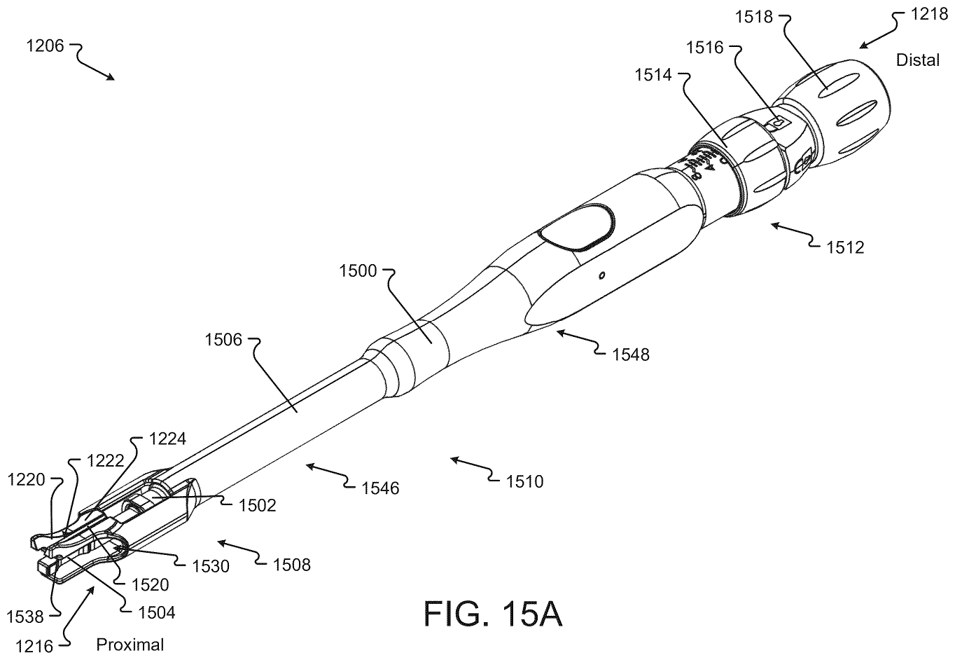

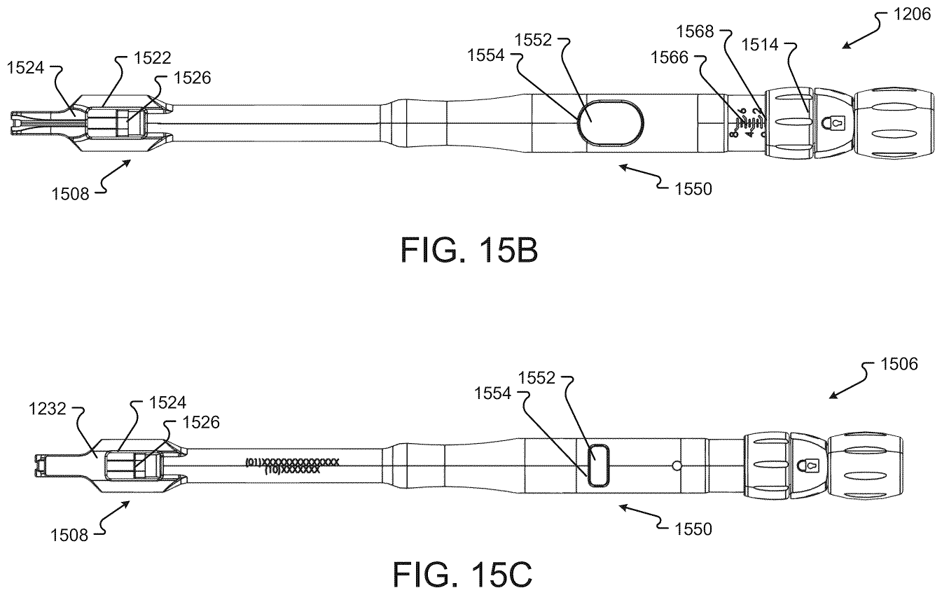

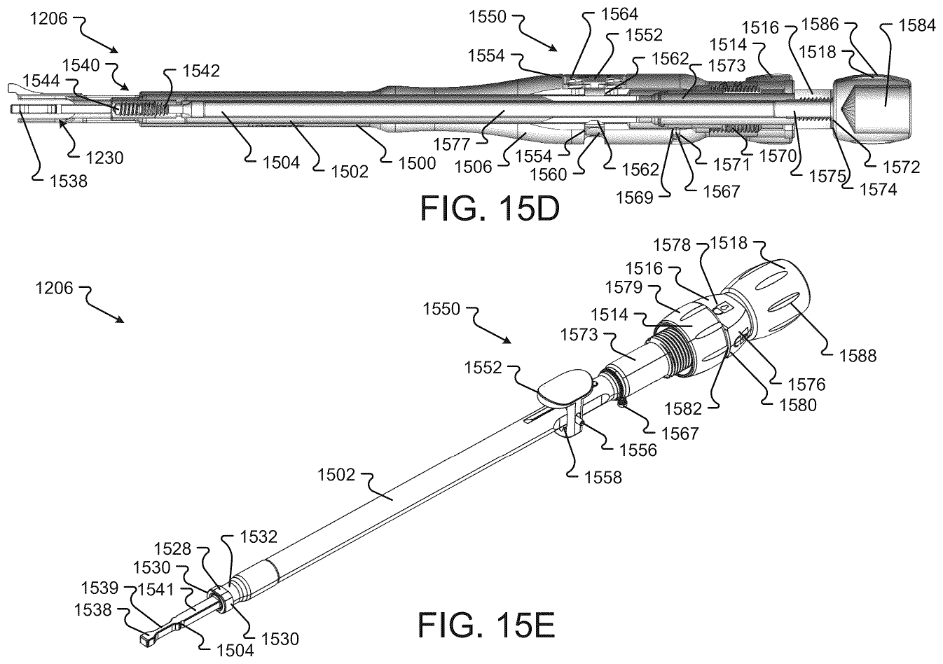

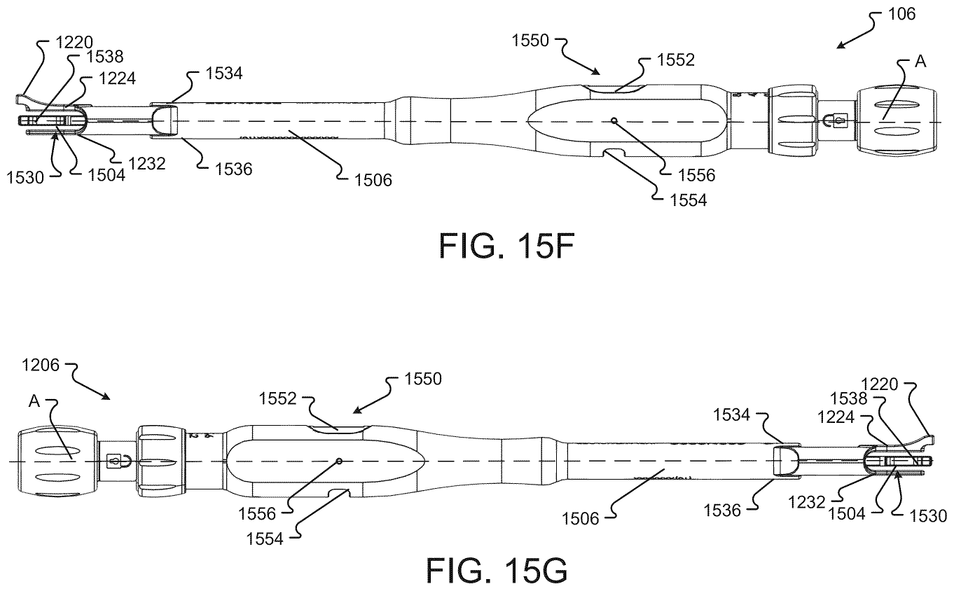

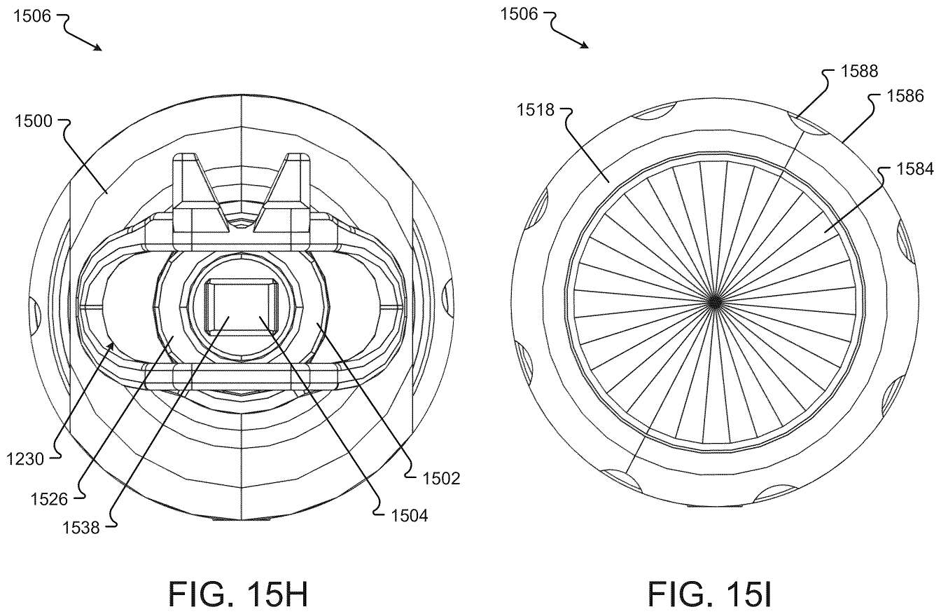

In some embodiments, as illustrated in FIG. 13E, the ledge 1340 terminates at the notch 1342. Unlike the cartridge 104 as illustrated in at least FIG. 2E, in which a portion 243 of the superior surface 206 is defined by the ledge 240 and the notch 242, the portion 1343 of the superior surface 1306 is only defined by the ledge 1340. Notably, the notch 1342 extends an amount into the superior surface 1306 from an exterior edge and toward a central point of the superior surface 1306 no further than the ledge 1340, such that a rear edge or surface 1345 of the ledge 1340 also forms a rear edge or surface 1347 of the notch 1342. As illustrated in FIGS. 15A-15H, engagement of these portions 1343 by the cartridge 1204 may reduce manufacturing costs, reduce the possibility of failure to disengage, and/or reduce the possibility of unseating the prosthesis 1202 by the cartridge 1204 after insertion into a patient at an implantation site.

In embodiments, the upper plate 1300 includes at least one set of teeth. The at least one set of teeth are configured to engage a contact surface of a respective (e.g., upper) vertebral body of the adjacent vertebral bodies between which the prosthesis 1202 is inserted during implantation, to prevent movement (e.g., sidewards and/or backwards movement) of the upper plate 1300 following implantation. In this regard, the at least one set of teeth may be considered anchoring teeth for the upper plate 1300.

For example, the at least one set of teeth includes first and second sets of teeth 1344 on the substantially flat or planar portion 1320 of the superior surface 1306. For instance, the first and second sets of teeth 1344 are aligned along the side edges 1334 and flank the convex portion 1318 of the superior surface 1306, being proximate to respective side edges 1334. It is noted, however, that the first and second sets of teeth 1344 may at least partially overlap with the convex portion 1318, without departing from the scope of the present disclosure. In addition, the first and second sets of teeth 1344 may be positioned between the front edge 1330 and the notch 1342 on the superior surface 1306.

It is noted that at least one tooth of the set of teeth 1344 may optionally form a portion of the front edge 1330, the side edge 1334, and/or the rear edge 1338 of the upper plate 1300. For example, the side edge 1334 of the upper plate 1300 may extend to an upper surface of the tooth of the set of teeth 1344. In addition, it is noted that a particular set of teeth 1344 may at least partially align with a particular cartridge engagement feature 1336. For example, the particular set of teeth 1344 may at least partially align with the notch 1342.

By way of another example, the at least one set of teeth includes a set of alignment teeth 1346. The set of alignment teeth 1346 may include at least two teeth that are aligned and positioned on the convex portion 1318 of the superior surface 1306. In some instances, the set of alignment teeth 1346 are positioned on a midline through the front edge 1330 and rear edge 1338 that is spaced substantially equally between the side edges 1334 of the upper plate 1300. This positioning allows the set of alignment teeth 1346 to be sighted with a midline of/on the upper vertebral body and/or a midline on the cartridge 1204 and/or the instrument 1206 during implantation.

It is noted that at least one tooth of the set of alignment teeth 1346 may optionally form a portion of the front edge 1330 or the rear edge 1338 of the upper plate 1300. For example, the chamfer 1328 of the upper plate 1300 may extend from the front edge 1330 to an upper surface of the tooth of the set of alignment teeth 1346.

In some non-limiting examples, the first and second sets of teeth 1344 and/or the set of alignment teeth 1346 may have teeth that are offset triangular prisms, with four sides that come to an offset point and a bottom on (or part of) the superior surface 1306. The teeth of the first and second sets of teeth 1344 and/or the set of alignment teeth 1346 may be substantially the same size or different sizes. For example, the teeth of the first and second sets of teeth 1344 and/or the set of alignment teeth 1346 may change in length, width, and/or height relative to the superior surface 1306 from the front edge 1330 to the rear edge 1338. For instance, the length, width, and/or height of the teeth of the first and second sets of teeth 1344 and/or the set of alignment teeth 1346 may increase from the front edge 1330 to the rear edge 1338. It is noted, however, that the teeth 1344 and/or teeth 1346 may not come to a point, but instead have sides that converge to a line or to a surface, without departing from the scope of the present disclosure.

In embodiments, the lower plate 1302 includes a substantially flat or planar superior surface 1310 and a substantially flat or planar inferior surface 1312. At least one protrusion 1348 may extend away from (e.g., upward) from the superior surface 1310. For example, a first protrusion 1348 may be positioned proximate to a first side edge 1354 of the lower plate 1302, and a second protrusion 1348 may be positioned proximate to a second side edge 1354 of the lower plate 1302.

In some non-limiting examples, the at least one protrusion 1348 may be approximately triangular prism in shape, with four sides that come to an offset point and a bottom on (or part of) the superior surface 1306. At least one side may be rounded, including a side that interfaces with a convex region of the insert 1304. The at least one protrusion 1348 may be substantially the same size or different sizes. For example, the at least one protrusion 1348 may change in length, width, and/or height relative to the superior surface 1306 depending on the amount of mobility in a particular direction to be provided by the prosthesis 1202.

In embodiments, the lower plate 1302 includes a chamfer 1350 on a front edge or leading edge 1352. For example, the chamfer 1350 may facilitate insertion of the prosthesis 1202 into a cavity defined between the adjacent vertebral bodies during implantation. Optionally, the lower plate 1302 includes a chamfer on one or more side edges 1354. For example, both side edges 1354 may include the chamfer. It is noted that the chamfer may facilitate insertion of the prosthesis 1202 into the cavity defined between the adjacent vertebral bodies during implantation.

In embodiments, the lower plate 1302 includes at least one cartridge engagement feature 1356 on a rear edge or trailing edge 1358. For example, the lower plate 1302 includes two opposing cartridge engagement features 1356 on the rear edge 1358. The at least one cartridge engagement feature 1356 includes a ledge 1360 within the inferior surface 1312 and a notch 1362 through the thickness of the lower plate 1302 and between the superior surface 1310 and the inferior surface 1312. In this regard, the at least one cartridge engagement feature 1356 may be considered to have a tiered structure or tiered configuration between the superior surface 1310, the ledge 1360, and the inferior surface 1312 of the lower plate 1302, for purposes of the present disclosure.

In some embodiments, as illustrated in FIG. 13F, the ledge 1360 terminates at the notch 1362. Unlike the cartridge 104 as illustrated in at least FIG. 2E, in which a portion 263 of the inferior surface 212 is defined by the ledge 260 and the notch 262, the portion 1363 of the inferior surface 1312 is only defined by the ledge 1360. Notably, the notch 1362 extends an amount into the inferior surface 1308 from an exterior edge and toward a central point of the inferior surface 1312 no further than the ledge 1360, such that a rear edge or surface 1365 of the ledge 1360 also forms a rear edge or surface 1367 of the notch 1362. As illustrated in FIGS. 15A-15H, engagement of these portions 1363 by the cartridge 1204 may reduce manufacturing costs, reduce the possibility of failure to disengage, and/or reduce the possibility of unseating the prosthesis 1202 by the cartridge 1204 after insertion into a patient at an implantation site.

It is noted that the ledges 1340, 1360 and the notches 1342, 1362 may be symmetrical, in some instances, between the upper plate 1300 and the lower plate 1302 about a plane through the insert 1304. In addition, it is noted that the ledges 1340, 1360 and the notches 1342, 1362 may be symmetrical, in some instances, on the upper plate 1300 and the lower plate 1302, respectively, about a plane through the upper plate 1300 and the lower plate 1302. As described in further detail with respect to the cartridge 1204, this symmetrical configuration may optionally allow for the cartridge 1204 to be used in multiple orientations, reducing the time necessary to prepare the system 1200 for use prior to or during an operation.

In embodiments, the lower plate 1302 includes at least one set of teeth. The at least one set of teeth are configured to engage a contact surface of a respective (e.g., lower) vertebral body of the adjacent vertebral bodies between which the prosthesis 1202 is inserted during implantation, to prevent movement (e.g., sidewards and/or backwards movement) of the lower plate 1302 following implantation. In this regard, the at least one set of teeth may be considered anchoring teeth for the lower plate 1302.

For example, the at least one set of teeth includes first and second sets of teeth 1364 on the inferior surface 1312. For instance, the first and second sets of teeth 1364 are aligned along the side edges 1354. In addition, the first and second sets of teeth 1364 may be positioned between the front edge 1352 and the rear edge 1358, such that the first and second sets of teeth 1364 extend on the inferior surface 1312 beyond the notches 1362 from the front edge 1352.

It is noted that at least one tooth of the set of teeth 1364 may optionally form a portion of the front edge 1352, the side edge 1354, and/or the rear edge 1358 of the lower plate 1302. For example, the side edge 1354 of the lower plate 1302 may extend from the front edge 1352 to a lower surface of the tooth of the set of teeth 1364.

By way of another example, the at least one set of teeth includes a set of alignment teeth 1366. The set of alignment teeth 1366 may include at least two teeth that are aligned and positioned on the inferior surface 1312. In some instances, the set of alignment teeth 1366 are positioned on a midline through the front edge 1352 and rear edge 1358 that is spaced substantially equally between the side edges 1352 of the lower plate 1302. This positioning allows the set of alignment teeth 1366 to be sighted with a midline of/on the lower vertebral body and/or a midline on the cartridge 1204 and/or the instrument 1206 during implantation.

It is noted that at least one tooth of the set of alignment teeth 1366 may optionally form a portion of the front edge 1352 or the rear edge 1358 of the lower plate 1302. For example, the chamfer 1350 of the lower plate 1302 may extend to a lower surface of the tooth of the set of alignment teeth 1366.

The first and second sets of teeth 1364 and/or the set of alignment teeth 1366 may have teeth that are offset triangular prisms, with four sides that come to an offset point and a bottom on the inferior surface 1312. The teeth of the first and second sets of teeth 1364 and/or the set of alignment teeth 1366 may be substantially the same size or different sizes. For example, the teeth of the first and second sets of teeth 1364 and/or the set of alignment teeth 1366 may increase in length, width, and/or height relative to the inferior surface 1312 from the front edge 1352 to the rear edge 1358. For instance, the length and height of the teeth of the first and second sets of teeth 1364 and/or the set of alignment teeth 1366 may increase from the front edge 1352 to the rear edge 1358. It is noted, however, that the teeth 1364 and/or teeth 1366 may not come to a point, but instead have sides that converge to a line or to a surface, without departing from the scope of the present disclosure.

As illustrated in FIGS. 13G and 13H, the sets of teeth 1344 and the sets of teeth 1364 may be in corresponding sets. For example, the corresponding sets may be offset (e.g., a different distance from respective side edges 1334, 1354), due to the positioning of the teeth 1344, 1364 relative to the respective notches 1342, 1362. However, it should be understood that the corresponding sets may be substantially aligned (e.g., a substantially same distance from respective side edges 1334, 1354) and not be offset, without departing from the scope of the present disclosure.

In some embodiments, the sets of teeth 1344 on the upper plate 1300 may have fewer teeth than the corresponding sets of teeth 1364 on the lower plate 1302. For example, the sets of teeth 1344 may each have four teeth, while the corresponding sets of teeth 1364 may each have five or six teeth. The additional number of teeth in the sets of teeth 1364 may cause the sets of teeth 1364 to substantially extend between the front edge 1352 and the rear edge 1358 of the lower plate 1302, such that at least a portion of teeth in the sets of teeth 1364 form a portion of the front edge 1352 and/or the rear edge 1358 of the lower plate 1302.

In some instances, at least one tooth of the sets of teeth 1344, 1346 may be shorter in height from the superior surface 1306 than an apex of the convex portion 1318 of the dome region of the superior surface 1306 of the upper plate 1300. In other instances, at least one tooth of the sets of teeth 1364, 1366 may be taller from the inferior surface 1312 of the lower plate 1302 than the teeth of the sets of teeth 1344, 1346 from the superior surface 1306 of the upper plate 1300.

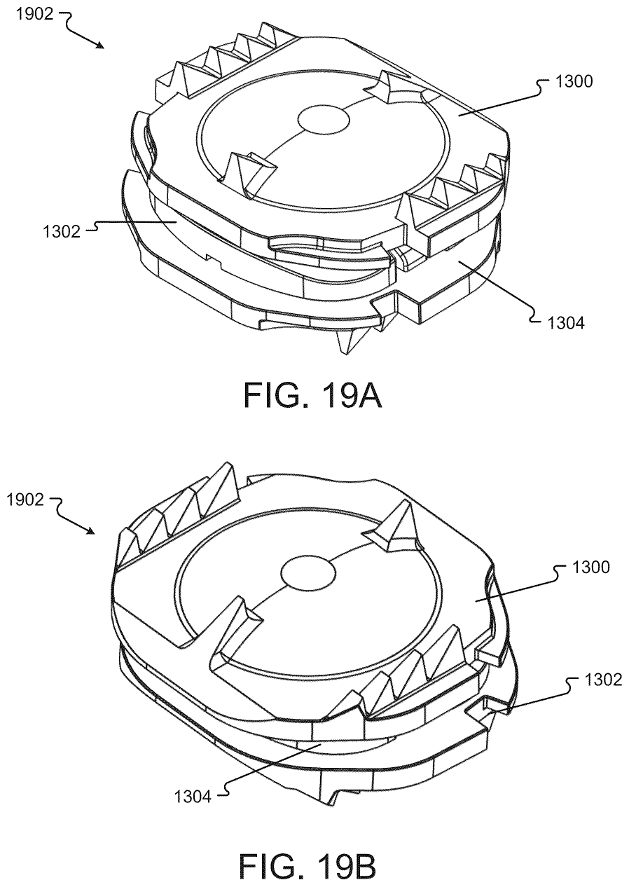

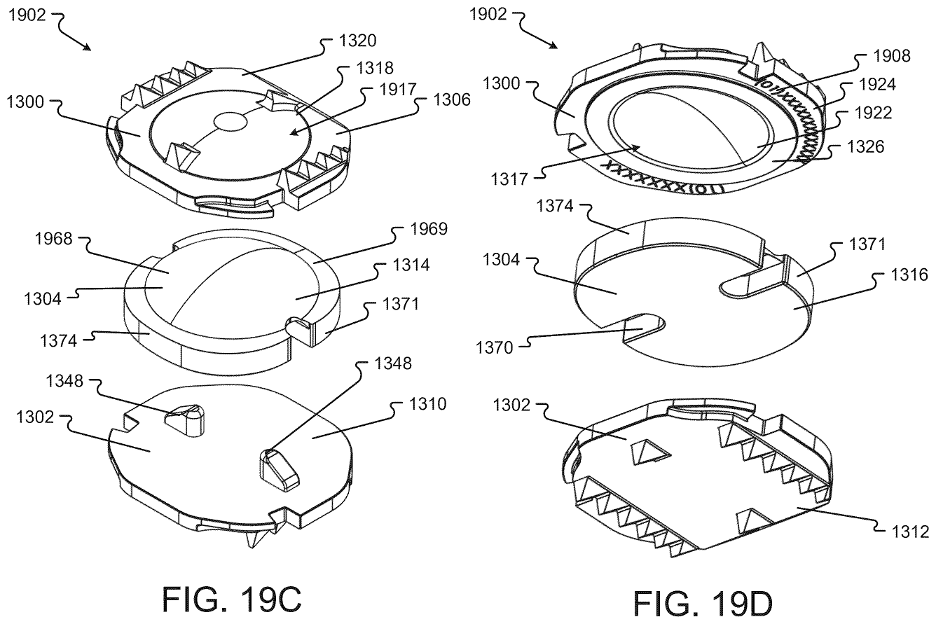

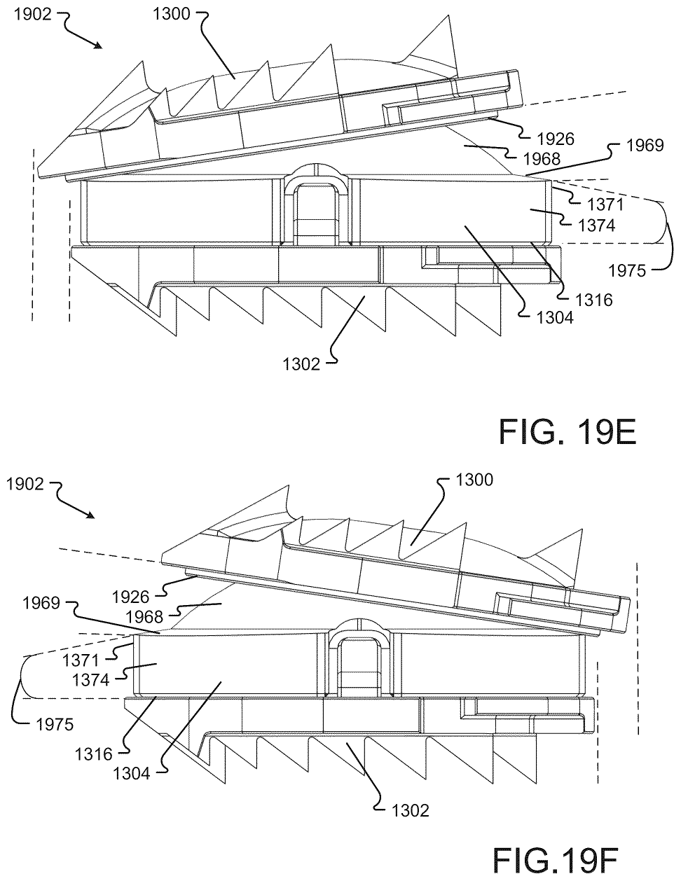

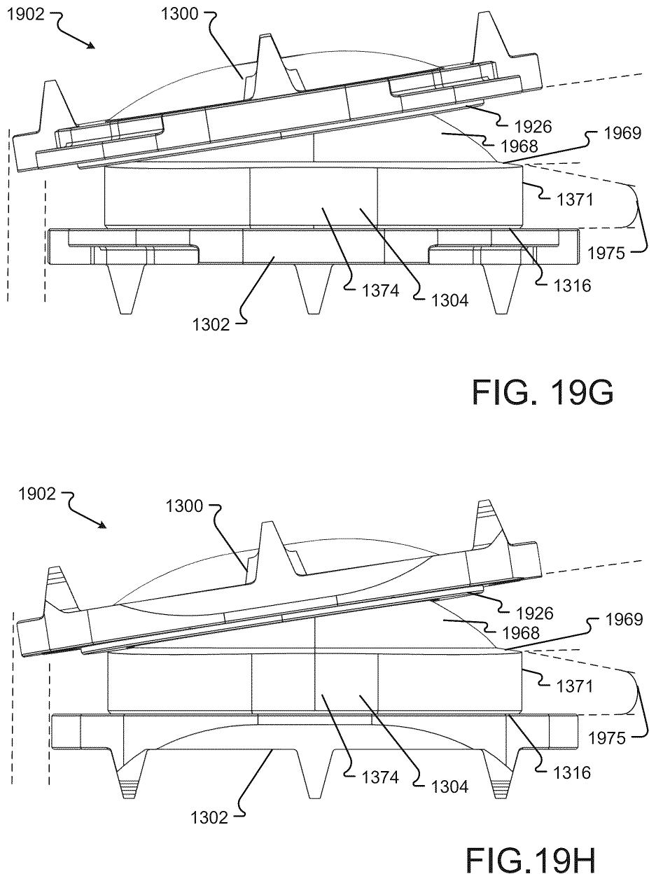

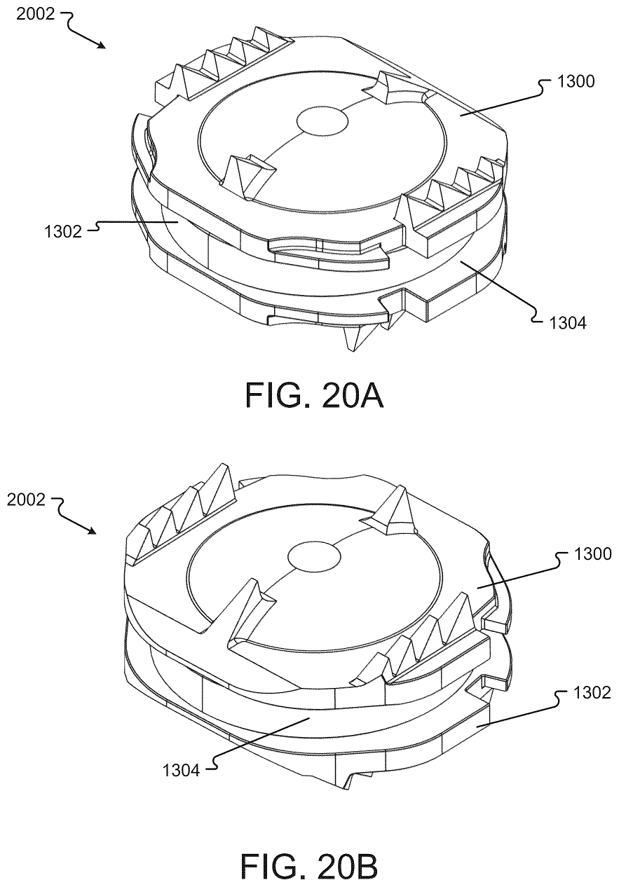

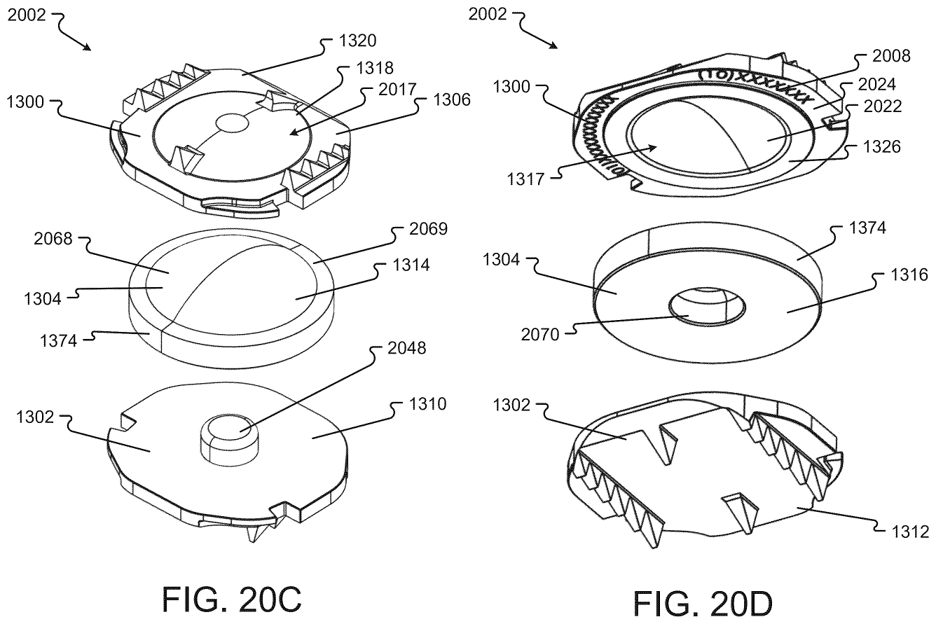

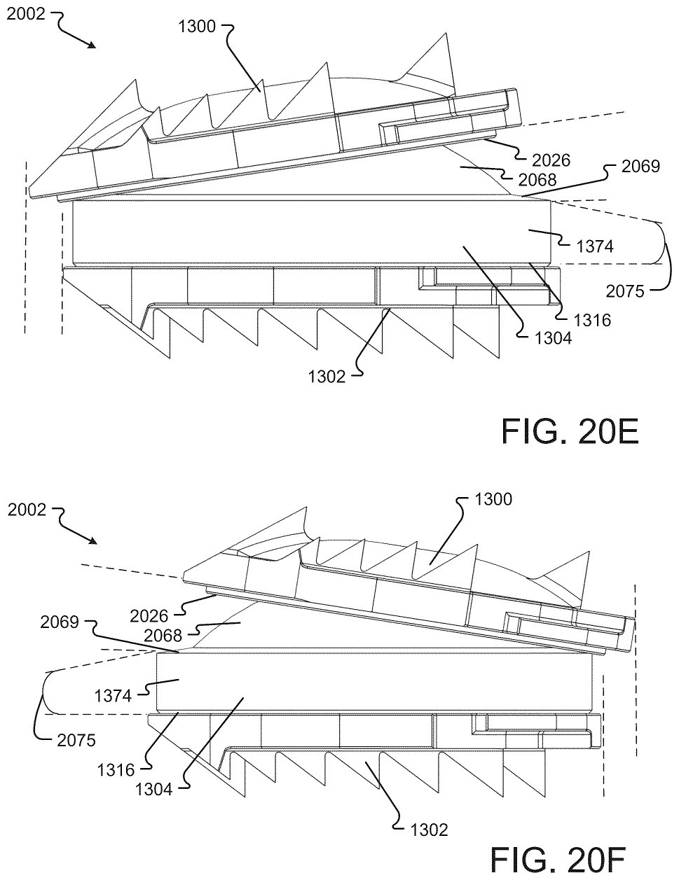

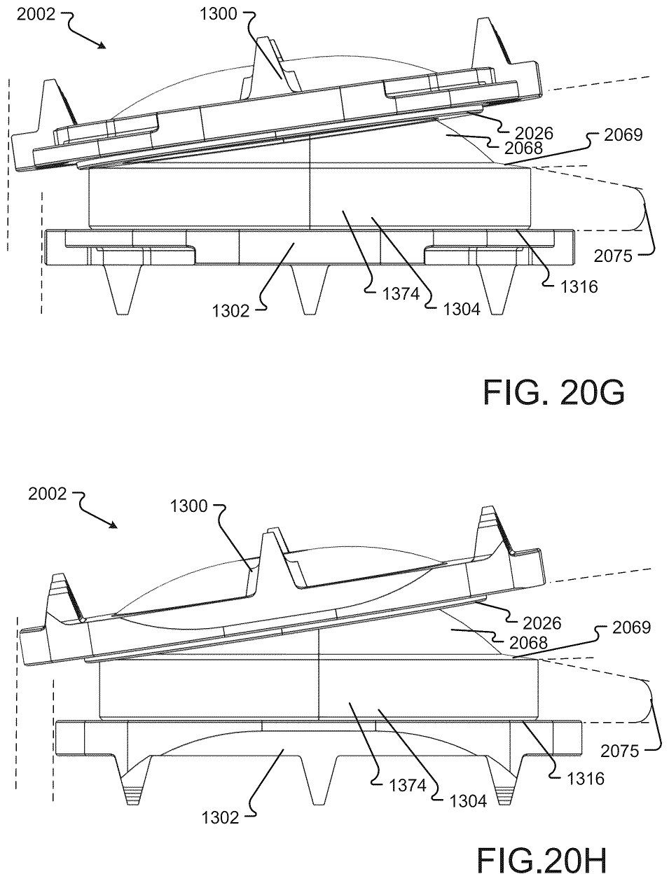

In embodiments, as illustrated in at least FIGS. 13C-13D and 13K-13L, at least a portion of the superior surface 1314 of the insert 1304 includes a convex and dome-shaped region 1368. For example, the convex region 1368 may be the entire superior surface 1314. Alternatively, the convex region 1368 may be a portion of the superior surface 1314 that is at least partially surrounded by a substantially flat or planar ring or flange portion having a decreased height relative to an apex of the convex region 1368. For example, the ring or flange portion may limit rotation in flexion/extension and lateral/bending directions. Where the superior surface 1314 includes the ring or flange portion, a transition between the convex region 1368 and the substantially flat or planar ring may be sloped and/or without a step-like change in height. The transition between the convex region 1368 and the substantially flat or planar ring may have a step-like change in height. Examples of this ring or flange portion are illustrated in variations of the prosthesis 1202 in FIGS. 19A-19F and FIGS. 20A-20F, as described in detail further herein.

In embodiments, at least a portion of the inferior surface 1316 of the insert 1304 is substantially flat or planar. At least one recess 1370 is positioned within the inferior surface 1316. For example, the at least one recess 1370 may extend inward toward a central point of the insert 1304, intersecting a sidewall 1371. The at least one recess 1370 is configured to receive a corresponding protrusion 1348 on the lower plate 1302, and extend through the entire thickness of the insert 1304 from the inferior surface 1316 to the superior surface 1314. For example, the at least one recess 1370 may have an exterior perimeter or cross-section that is contoured to conform to an exterior perimeter or cross-section of the corresponding protrusion 1348. It is noted that the recesses 1370 extending through the entire thickness of the insert 1304 may assist in retaining the insert 1304 between the plates 1300, 1302, as the insert 1304 may have to separate from the lower plate 1302 an amount corresponding to a height of the protrusion 1348 and the corresponding recess 1370.

Although the at least one recess 1370 is shown as intersecting the sidewalls 1371 of the insert 1304, it should be understood that the insert 1304 may be dimensioned to have the at least one recess 1370 surrounded by material, such that the corresponding protrusion 1348 is embedded within the material of the insert 1304, without departing from the scope of the present disclosure.

In some configurations, the at least one recess 1370 extends through the entirety of the thickness of the insert 1304, from the inferior surface 1316 to the superior surface 1314. In other configurations, depending on the height of the corresponding protrusion 1348, the at least one recess 1370 may only extend into the inferior surface 1316 a predetermined depth and not entirely through the thickness to the superior surface 1314 (e.g., similar to the configuration of the protrusion 248 and the recess 270 of the prosthesis 102).

As illustrated in a comparison between FIGS. 13K and 13K and FIGS. 2K and 2L, it is noted that the corresponding recess 1370 may be substantially similar in dimension (e.g., is closer in tolerance) to the respective protrusion 1348 as compared to the recess 270 and the protrusion 248 of the insert 204 of the prosthesis 102. In some configurations, the closeness in tolerancing between the at least one protrusion 1348 and the corresponding recess 1370 may be desirable to limit play and/or displacement between the insert 1304 and the lower plate 1302 of the prosthesis 1202, as compared to the greater tolerancing between the protrusion 248 of the lower plate 202 and the recess 270 of the insert 204 of the prosthesis 102 (e.g., as illustrated in FIGS. 2K and 2L). Limiting movement between the insert 1304 and the lower plate 1302 subsequently limits mobility of the spine at the adjacent vertebral bodies between which the prosthesis 1202 is implanted.

However, it is contemplated that, more similar to the recess 270 for the protrusion 248 of the prosthesis 102, the recess 1370 may be larger than the protrusion 1348 to allow for increased adjustment of the insert 1304 relative to the lower plate 1302, without departing from the scope of the present disclosure. In addition, it is contemplated that, more similar to the recess 1370 for the protrusion 1348 of the prosthesis 1202, the recess 270 may be more closely toleranced to conform to the dimensions of the protrusion 248 to allow for limit movement of the insert 204 relative to the lower plate 202, without departing from the scope of the present disclosure.

In embodiments, the inclusion of multiple protrusions 1348 increases a width of the insert 1304 such that a cross-section of the insert 1304 (when viewed from the superior surface 1314) has a longer major axis in the width direction and a shorter minor axis in the length direction (or direction of insertion).

Optionally, one or more grooves 1372 may extend from an endwall 1374 and/or the sidewall 1371 of the insert 1304 in the inferior surface 1316. For example, the at least one recess 1370 may extend from a front endwall 1374 to a rear endwall 1374 of the insert 1304. In general, the one or more grooves 1372 may extend between any two side surfaces and/or end surfaces of the insert 1304. However, it is contemplated that the one or more grooves 1372 may be positioned within the inferior surface 1316, without extending to a particular sidewall 1371 and/or endwall 1374 of the insert 1304, without departing from the scope of the present disclosure.

It is noted that were there are multiple grooves 1372, the grooves 1372 may intersect or be isolated within the inferior surface 1316, without departing from the scope of the present disclosure. In addition, it is noted that there may be a central groove 1372, from which other grooves 1372 radiate to sidewalls 1371 and/or endwalls 1374 of the insert 1304, without departing from the scope of the present disclosure. The one or more grooves 1372 may assist in the flow of fluids (e.g., that form within a central groove 1372) into the cavity defined between the upper plate 1300 / lower plate 1302 (and thus the cavity defined between the adjacent vertebral bodies in which the prosthesis 1202 is implanted). The one or more grooves 1372 may additionally or alternatively assist in the movement of the insert 1304 relative to the lower plate 1302, by adding fluid flow between the insert 1304 and the lower plate 1302 and reducing the possibility the inferior surface 1316 of the insert 1304 sticks to the superior surface 1310 of the lower plate 1302.

In some instances, the protrusion 1348 and/or the at least one recess 1370 have a depth that is at least a height of the sidewall 1371 and/or the endwall 1374. In other instances, the protrusion 1348 and/or the recess 1370 have a depth greater than the height of the sidewall 1371 and/or the endwall 1374.

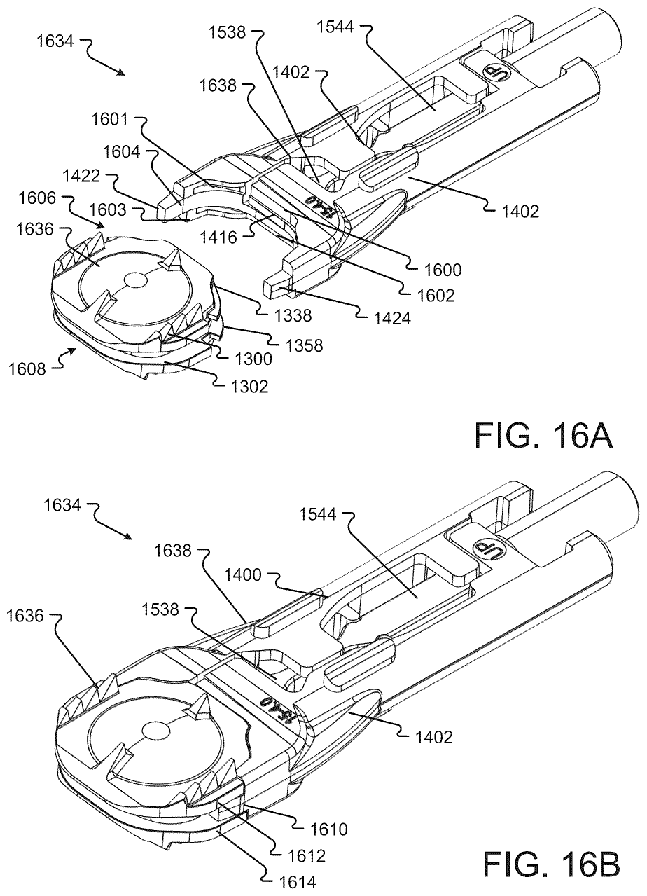

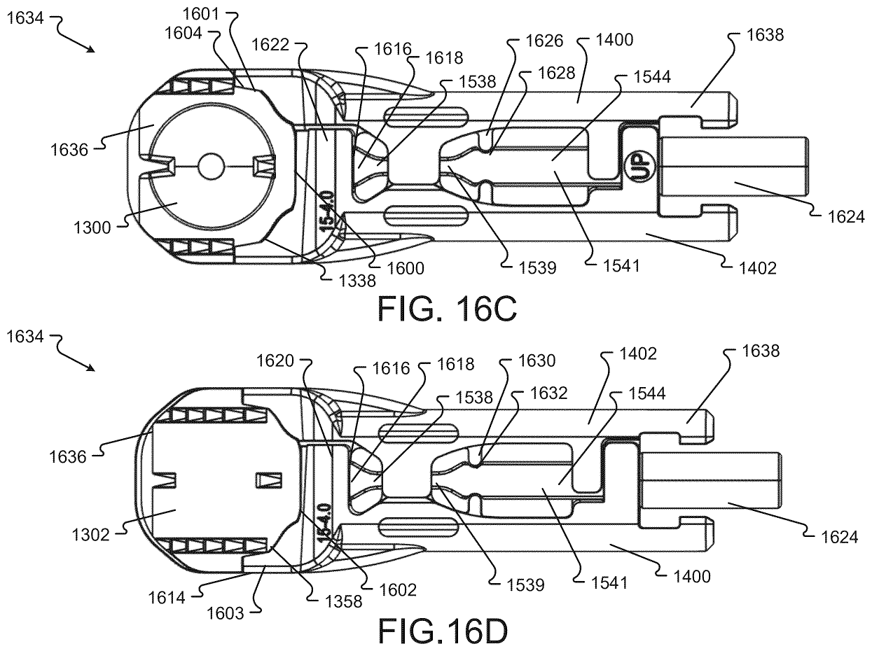

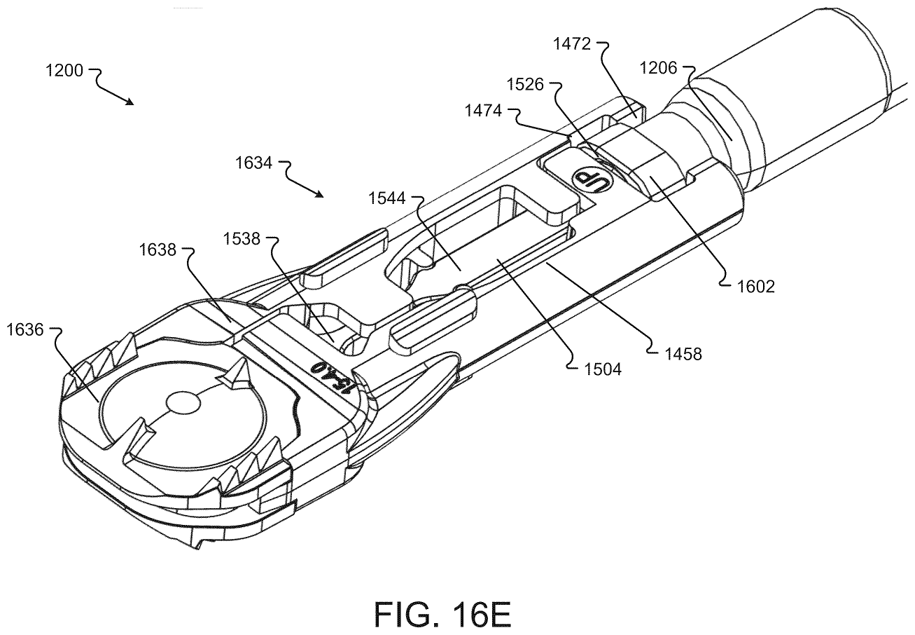

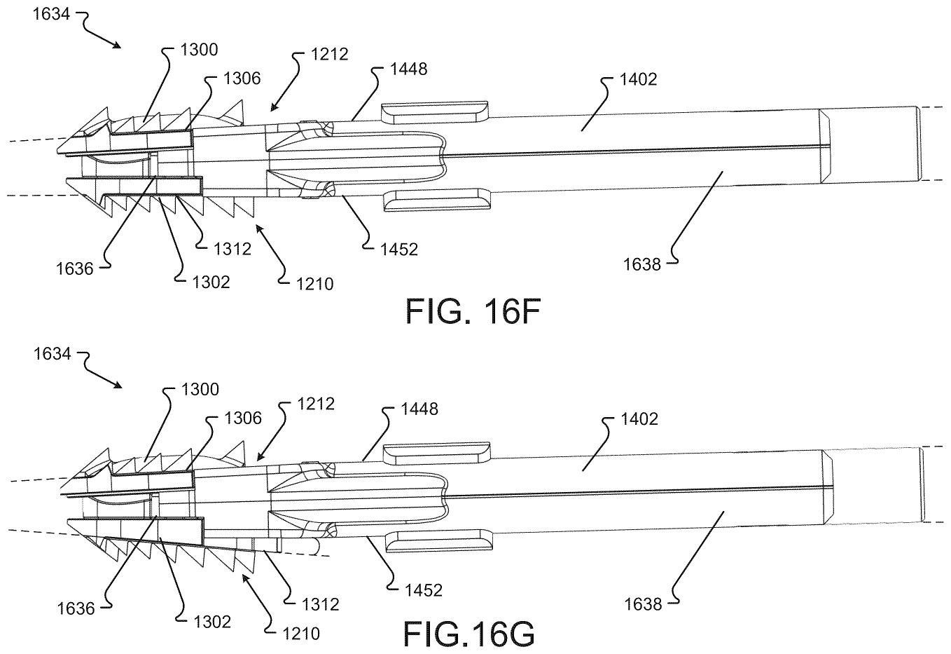

As illustrated in FIGS. 13I-13J, one contemplated natural state for the prosthesis 1202 includes having the upper plate 1300 offset slightly forward of, and tilted slightly toward, the lower plate 1302. For example, the upper plate 1300 may be offset from the lower plate 1302 by approximately 0.5 mm. In addition, the upper plate 1300 may be tilted toward the lower plate 1302 by approximately 3°. It is noted, however, that this is only an exemplary state, and that other natural states are contemplated, without departing from the scope of the present disclosure. For example, FIGS. 16F and 16G illustrate variations between a standard configuration and a lordotic configuration, respectively, for the prosthesis 1202.

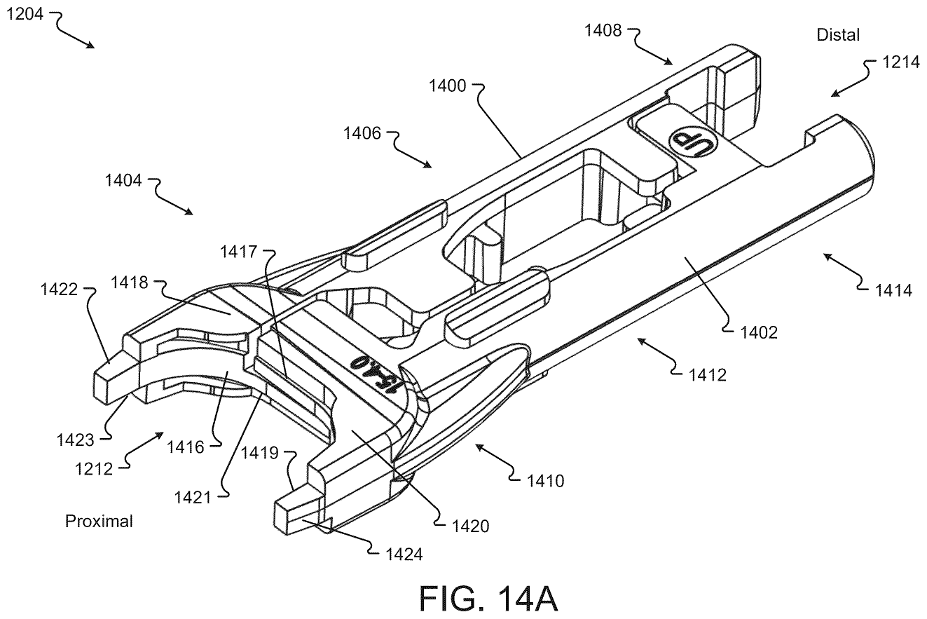

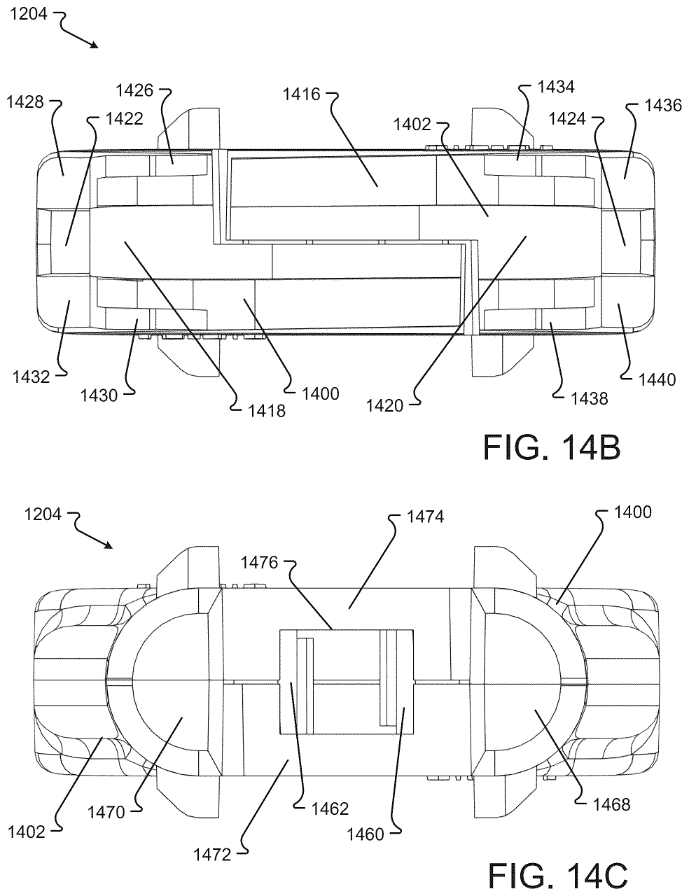

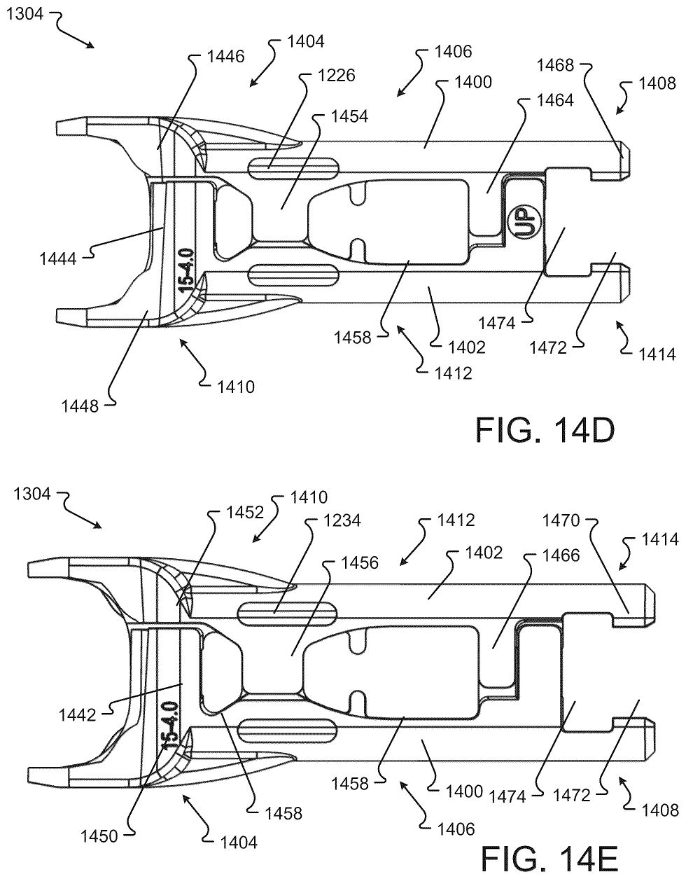

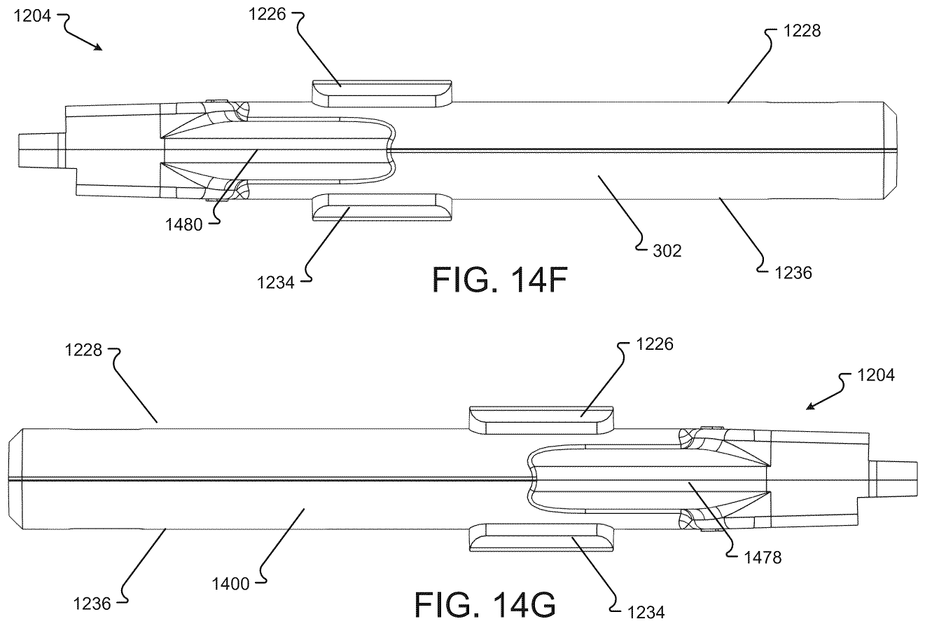

FIGS. 14A-14G in general illustrate the cartridge 1204 of the system 1200, in accordance with one or more embodiments of the present disclosure. It should be understood that any embodiment of the cartridge 1204 illustrated in FIGS. 12A-12G may be directed to the cartridge 1204 illustrated in FIGS. 14A-14G, and vice versa, without departing from the scope of the present disclosure.

In embodiments, the cartridge 1204 includes a first arm 1400 and a second arm 1402. The first arm 1400 has a proximal section 1404, a middle section 1406, and a distal section 1408. The second arm 1402 has a proximal section 1410, a middle section 1412, and a distal section 1414. The proximal section 1404 of the first arm 1400 and the proximal section 1410 of the second arm 1402 form the proximal end 1212 of the cartridge 1204. The distal section 1408 of the first arm 1400 and the distal section 1414 of the second arm 1402 form the distal end 1214 of the cartridge 1204.

In embodiments, the proximal end 1212 of the cartridge 1204 includes a receiver 1416, formed from a first hand 1418 on the first arm 1400 and a second hand 1420 on the second arm 1402. The first hand 1418 includes a first finger 1422. The second hand 1420 includes a second finger 1424.

In embodiments, the receiver 1416 includes one or more engagement features configured to engage the prosthesis 1202. For example, the receiver 1416 engages the prosthesis 1202 on at least one of the rear edge 1338 and/or the side edges 1334 of the upper plate 1300. By way of another example, the receiver 1416 engages the prosthesis 1202 on at least one of the rear edge 1358 and/or the side edges 1354 of the lower plate 1302.

For instance, the arm 1400 and the arm 1402 have a first edge 1417 of the receiver 1416, where the first edge 1417 has one or more substantially straight portions 1419 at the proximal end of a hand 1418 or hand 1420, as compared to the one or more portions 501 of the first edge 500 of the cartridge 104 (e.g., as illustrated in at least FIGS. 5B-5D). The portions 1419 of the first edge 1417 of the receiver 1416 that are contoured to conform to the rear edge 1338 of the upper plate 1300, and specifically that are contoured to conform to the engagement portions 1343 along the rear edge 1338, may reduce manufacturing costs, reduce the possibility of failure to disengage, and/or reduce the possibility of unseating the prosthesis 1202 by the cartridge 1204 after insertion into a patient at an implantation site.

In addition, the arm 1400 and the arm 1402 have a second edge 1421 of the receiver 1416, where the second edge 1421 has one or more substantially straight portions 1423 at the proximal end of a hand 1418 or hand 1420, as compared to the one or more portions 503 of the second edge 502 of the cartridge 104 (e.g., as illustrated in at least FIGS. 5B-5D). The portions 1423 of the second edge 1421 of the receiver 1416 that are contoured to conform to the rear edge 1358 of the lower plate 1302, and specifically that are contoured to conform to the engagement portions 1363 along the rear edge 1358, may reduce manufacturing costs, reduce the possibility of failure to disengage, and/or reduce the possibility of unseating the prosthesis 1202 by the cartridge 1204 after insertion into a patient at an implantation site.

In embodiments, the engagement features of the cartridge 1204 include shelves that correspond to ledges on the prosthesis 1202, to assist in securing the prosthesis 1202 within the receiver 1416 of the cartridge 1204. For example, the first hand 1418 includes an upper shelf 1426 that is positionable over a first ledge 1340 of the upper plate 1300. By way of another example, the first hand 1418 additionally includes a lower shelf 1430 that is positionable under a first ledge 1360 of the lower plate 1302. By way of another example, the second hand 1420 includes an upper shelf 1434 that is positionable over a second ledge 1340 of the upper plate 1300. By way of another example, the second hand 1420 includes a lower shelf 1438 that is positionable under a second ledge 1360 of the lower plate 1302.

Optionally, the engagement features of the cartridge 1204 include tabs that correspond to notches in the prosthesis 1202, to assist in securing the prosthesis 1202 within the receiver 1416 of the cartridge 1204. For example, the first hand 1418 includes an upper tab 1428 that is positionable within a first notch 1342 of the upper plate 1300. By way of another example, the first hand 1418 additionally includes a lower tab 1432 that is positionable within a first notch 1362 of the lower plate 1302. By way of another example, the second hand 1420 includes an upper tab 1436 that is positionable within a second notch 1342 of the upper plate 1300. By way of another example, the second hand 1420 includes a lower tab 1440 that is positionable within a second notch 1362 of the lower plate 1302.

It is noted that, in some configurations, a symmetry between the fingers 1422, 1424, the shelves 1426, 1430, 1434, 1438 and the ledges 1340, 1360, and the tabs 1428, 1432, 1436, 1440 and the notches 1342, 1362, allow for the cartridge 1204 engage the prosthesis 1202 in either a first orientation or a second orientation, reducing the time needed for assembly of the prosthesis 1202 and the cartridge 1204 prior to or during an operation. However, this is possible only where the natural state is not the exemplary offset and tilted configuration of the upper plate 1300 and the lower plate 1302, as described previously herein. Where the natural state is the exemplary offset and tilted configuration, the shelves 1426, 1430, 1434, 1438 and the tabs 1428, 1432, 1436, 1440 may be positioned on the first hand 1418 and the second hand 1420 accordingly.

As illustrated in FIGS. 14D and 14E, the proximal section 1404 of the first arm 1400 includes a proximal crossmember 1442, and the proximal section 1410 of the second arm 1402 includes a proximal crossmember 1444. In some embodiments, the proximal crossmember 1442 and the proximal crossmember 1444 are each of a thickness that is less than a total thickness of the respective first arm 1400 and second arm 1402. For example, the thicknesses of the crossmembers 1442, 1444 may be such that, when the cartridge 1204 is assembled and the crossmembers 1442, 1444 are overlapping, an upper surface 1446 of the first arm 1400 and an upper surface 1448 of the second arm 1402 (e.g., that together form the upper surface 1228 of the cartridge 1204) are substantially flush or planar. In addition, the thicknesses of the crossmembers 1442, 1444 may be such that, when the cartridge 1204 is assembled and the crossmembers 1442, 1444 are overlapping, a lower surface 1450 of the first arm 1400 and a lower surface 1452 of the second arm 1402 (e.g., that together form the lower surface 1236 of the cartridge 1204) are substantially flush or planar.

It is noted that the upper wedges 1226 are on the upper surface 1446 of the first arm 1400 and the upper surface 1448 of the second arm 1402. In addition, the lower wedges 1234 are on the lower surface 1450 of the first arm 1400 and the lower surface 1452 of the second arm 1402.

In embodiments, the middle section 1406 of the first arm 1400 includes a middle crossmember 1454, and the middle section 1412 of the second arm 1402 includes a middle crossmember 1456. In some embodiments, the middle crossmember 1454 and the middle crossmember 1456 are each of a thickness that is less than a total thickness of the respective first arm 1400 and second arm 1402. For example, the thicknesses of the crossmembers 1454, 1456 may be such that, when the cartridge 1204 is assembled and the crossmembers 1454, 1456 are overlapping, the upper surface 1446 of the first arm 1400 and the upper surface 1448 of the second arm 1402 (e.g., that together form the upper surface 1228 of the cartridge 1204) are substantially flush or planar. In addition, the thicknesses of the crossmembers 1454, 1456 may be such that, when the cartridge 1204 is assembled and the crossmembers 1454, 1456 are overlapping, the lower surface 1450 of the first arm 1400 and the lower surface 1452 of the second arm 1402 (e.g., that together form the lower surface 1236 of the cartridge 1204) are substantially flush or planar.

In some embodiments, a cavity 1458 may be at least partially defined within the proximal sections 1404, 1410 and/or at least partially defined within the middle sections 1406, 1412 of the first arm 1400 and the second arm 1402, respectively. A portion of the instrument 1206 (e.g., a locking pin 1544 with an expansion bit 1538) may be inserted to engage with the cartridge 1204 and cause the cartridge 1204 to release the prosthesis 1202. For example, the crossmember 1454 may include a release tab 1460, and the crossmember 1456 may include a release tab 1462. As described in detail further herein, the portion of the instrument 1206 (e.g., the locking pin 1544 with the expansion bit 1538) engages the release tabs 1460, 1462 to cause the cartridge 1204 to release the prosthesis 1202 after the prosthesis 1202 is implanted at the implantation site.

It is noted that the cartridge 1204 may only require one of the release tabs 1460, 1462 to disengage the cartridge 1204 from the prosthesis 1202 via interaction with the portion of the instrument 1206, without departing from the scope of the present disclosure.

In some configurations, the cartridge 1204 and the locking pin 1544 with the expansion bit 1538 may be pre-packaged together as a kit or assembly. In additional configurations, the prosthesis 1202, the cartridge 1204, and the locking pin 1544 with the expansion bit 1538 may be pre-packaged together as a kit or assembly. In further configurations, the prosthesis 1202 and the cartridge 1204 may be pre-packaged together as a kit or assembly. This pre-packaging may reduce the possibility of interference with (e.g., damaging, misaligning, and the like) the assembly of the prosthesis prior to insertion into the patient, while promoting sterility and the interchangeability of the assembly and a trial implant 1700. For example, different trial implants 1700 may be trialed with the same instrument 1206, and then a kit or assembly including the prosthesis 1202, the cartridge 1204, and/or the locking pin 1544 with the expansion bit 1538 that matches a determined trial implant 1700 may be installed within the instrument 1206 for implantation.

In embodiments, the distal section 1408 of the first arm 1400 includes a distal crossmember 1464, and the distal section 1414 of the second arm 1402 includes a distal crossmember 1466. In some embodiments, the distal crossmember 1464 and the distal crossmember 1466 are each of a thickness that is less than a total thickness of the respective first arm 1400 and second arm 1402. For example, the thicknesses of the crossmembers 1464, 1466 may be such that, when the cartridge 1204 is assembled and the crossmembers 1464, 1466 are at least partially overlapping, the upper surface 1446 of the first arm 1400 and the upper surface 1448 of the second arm 1402 (e.g., that together form the upper surface 1228 of the cartridge 1204) are substantially flush or planar. In addition, the thicknesses of the crossmembers 1464, 1466 may be such that, when the cartridge 1204 is assembled and the crossmembers 1464, 1466 are overlapping, the lower surface 1450 of the first arm 1400 and the lower surface 1452 of the second arm 1402 (e.g., that together form the lower surface 1236 of the cartridge 1204) are substantially flush or planar.

As illustrated in FIGS. 14B-14C and 14F-14G, in some embodiments the upper surface 1228 and the lower surface 1236 of the cartridge 1204 are respectively substantially flat or planar along the entire length of the cartridge 1204, aside from the wedges 1226, 1234 that extend from the respective surfaces 1228, 1236. In some instances, a total height of the upper wedges 1226 from the upper surfaces 1446, 1448 is equal to or less than a height of the tallest tooth of the sets of teeth 1344, 1346 on the upper plate 1300 (e.g., as illustrated in FIGS. 16A-16E). In other instances, a total height of the lower wedges 134 from the upper surfaces 1450, 1452 is equal to or less than a height of the tallest tooth of the sets of teeth 1364, 1366 on the lower plate 1302 (e.g., as illustrated in FIGS. 16A-16E). This allows for a reduced size of implantation site in the patient, as the surfaces 1446, 1448, 1450, 1452 with respective wedges 1226, 1234 do not extend outside of a height envelope defined by the respective sets of teeth 1344, 1346, 1364, 1366.

The distal section 1408 additionally includes a first tail 1468, and the distal section 1414 additionally includes a second tail 1470. The space between the first tail 1468 and the second tail 1470 defines a slot 1472 of the distal end 1214 of the cartridge 1204. In addition, the crossmembers 1464, 1466 and the tails 1468, 1470 define a cavity 1474 of the distal end 1214 of the cartridge 1204.

In some embodiments, the cavity 1474 has a greater width than the slot 1472. As described in detail further herein, a portion of the instrument 1206 (e.g., a coupler 1526) may be inserted within the slot 1472 and re-oriented once located within the cavity 1474 to mate with the cavity 1474. When in the first orientation, the portion of the instrument 1206 (e.g., the coupler 1526) may be removed from the cavity 1474 and the slot 1472. When in the second orientation, the portion of the instrument 1206 (e.g., the coupler 1526) is retained within the cavity 1474 by the slot 1472, due to the mating between the coupler 1526 and the cavity 1474.

The crossmembers 1464, 1466 may each be configured to collectively define an opening 1476 in the distal end of the cartridge 1204. In some embodiments, the opening 1476 provides access to a portion of the instrument 1206 (e.g., the locking pin 444 with the expansion bit 438) into the cavity 1458 for engagement with the release tabs 1460, 1462. In embodiments where the locking pin 1544 with the expansion bit 1538 is pre-packaged with the cartridge 1204 (and the prosthesis 1202), the expansion bit 1538 may be at least partially positioned within the cavity 1474, such that the coupler 1526 may receive the expansion bit 1538 when inserted through the slot 1472.

In some embodiments, the first arm 1400 may include a support member 1478 between the proximal section 1404 and the middle section 1406. In addition, the second arm 1402 may include a support member 1480 between the proximal section 1410 and the middle section 1412. In some instances, the support members 1478, 1480 are dimensioned to be no wider than the width of the proximal end 1212 of the cartridge 1204. This reduces the total footprint width of the combined prosthesis 1202 and cartridge 1204, reducing the width of the implantation site needed during the operation.

FIGS. 15A-15I in general illustrate the instrument 1206 of the system 1200, in accordance with one or more embodiments of the present disclosure. It should be understood that any embodiment of the instrument 1206 illustrated in FIGS. 12A-12G may be directed to the instrument 1206 illustrated in FIGS. 15A-15I, and vice versa, without departing from the scope of the present disclosure.

In embodiments, the instrument 1206 includes an outer housing 1500, a locking shaft 1502 (or middle shaft), and a release driver 1504 (or inner shaft). The outer housing 1500 includes a body 1506 that extends from the proximal end 1216 to the distal end 1218 of the instrument 1206. The body 1506 includes a proximal section 1508, a middle section 1510, and a distal section 1512.

The outer housing 1500 is connected to an actuator 1514. The actuator 1514 (e.g., proximate to the distal end 1218) of the instrument 1206 causes a displacement of depth stops 1220 on the upper tab 1224 of the body 1506 of the instrument 1206 relative to the cartridge 1204 when the cartridge 1204 is inserted within the opening 1230 of the instrument 1206. For example, the depth stops 1220 may be displaced along a longitudinal axis A through the instrument 1206 via actuation of the actuator 1514, to assist in the positioning of the prosthesis 1202 with the cartridge 1204 between adjacent vertebral bodies, as described in detail further herein.

In addition, the locking shaft 1502 passes through the outer housing 1500 and is connected to an actuator 1516. For instance, the actuator 1516 (e.g., proximate to the distal end 1218) of the instrument 1206 causes the locking shaft 1502 to receive and engage the cartridge 1204 when the cartridge 1204 is inserted into the opening 1230 of the instrument 1206, as described in detail further herein.

Further, the release driver 1504 passes through the locking shaft 1502 and is connected to an actuator 1518. The actuator 1518 (e.g., proximate to the distal end 1218) of the instrument 1206 actuates the release driver 1504 to cause the cartridge 1204 to disengage from the prosthesis 1202, as described in detail further herein.

Although separate exemplary actuators 1514, 1516, 1518 are illustrated, it should be understood that the instrument 1206 may be configured such that the same actuator 1514, 1516, and/or 1518 performs multiple of the described operations, without departing from the scope of the present disclosure.

In the proximal section 1508, the instrument 1206 includes the upper tab 1224 with the channel 1222 defined by at least one depth stop 1220, and the lower tab 1232. The upper tab 1224 includes an alignment feature 1520 that aligns with complementary features on the prosthesis 1202 and/or the cartridge 1204. For example, the alignment feature 1520 may be a groove, a recess, a protrusion, a mark (e.g., laser-etched, drawn, an applied sticker, or the like), colors, and/or patterns or another feature that aligns with complementary protrusions (e.g., teeth) or indentations on the prosthesis 1202 and/or the cartridge 1204, and/or protrusions or indentations on a proximate vertebral body. For instance, the shape of the channel 1222 and/or the location of the alignment feature 1520 may correspond to a midline of the adjacent vertebral bodies between which the prosthesis 1202 is being implanted. In this regard, a user (e.g., a surgeon, or other medical professional) may utilize the combination of the alignment feature 1520 and the complementary features on the cartridge 1204 and/or the prosthesis 1202, along with any optional marks on the adjacent vertebral bodies, to sight the prosthesis 1202 during insertion between the adjacent vertebral bodies at the implantation site and/or after during post-operative monitoring.

In some embodiments, such as where the alignment feature 1520 is a groove or recess, the alignment feature 1520 can optionally assist in positioning an osteotome 602 (e.g., as illustrated in FIGS. 6A-6G and 8) within the channel 1222 between the depth stops 1220 on the upper tab 1224 of the instrument 1206, as previously described herein. For example, this may be beneficial where the proximal end 1216 of the instrument 1206 is not visible as it is inserted within the implantation site, such that visual confirmation of the appropriate position of the osteotome 602 within the channel 1222 is not available.

As illustrated in FIGS. 15B and 15C, the proximal section 1508 of the body 1506 includes at least one of a first window 1522 in the upper tab 1224 of the instrument and a second window 1524 in the lower tab 1232 of the instrument 1206. A coupler 1526 of the locking shaft 1502 is visible within the windows 1522, 1524 (e.g., being positioned at a proximal end of the locking shaft 1502), and the mating of the coupler 1526 to the cartridge 1204 and/or a trial implant 1700 is visible through the windows 1522, 1524.

In some non-limiting examples, as illustrated in FIG. 15E, the coupler 1526 and the locking shaft 1502 form a T-shape, with locking shaft 1502 for the long leg of the T-shape and the coupler 1526 including a middle section 1528 and a pair of lobes 1530 for the short leg of the T-shape. A width of the coupler 1526 is such that the coupler 1526 may fit through a width of the slot 1472 at the distal end 1214 of the cartridge 1204 when the coupler 1526 is in a first orientation. In addition, a length of the coupler 1526 is such that the coupler 1526 may be retained within the cavity 1474 when the coupler 1526 is in a second orientation, as the length of the coupler 1526 is greater than the width of the slot 372.

In additional non-limiting examples, the locking shaft 1502 may include a slot 1532 that is decreased in diameter as compared to the length of the coupler 1526. For instance, the slot 1532 may correspond to the tails 1468, 1470 of the cartridge 1204, to allow for rotation of the locking shaft 1502 within the slot 1472 at the distal end 1214 of the cartridge 1204 when the coupler 1526 is positioned within the cavity 1474 at the distal end 1214 of the cartridge 1204.

In further non-limiting examples, a thickness of the coupler 1526 is approximately a thickness of the distal end 1214 of the cartridge 1204, such that the coupler 1526 and the distal end 1214 of the cartridge 1204 are approximately equal in height when the coupler 1526 is in the second orientation. It is noted that the window 1522 may allow for the rotation between the first orientation and the second orientation, such as where the length of the coupler 1526 may be greater than the thickness of the cartridge 1204 and/or a thickness between an upper surface 1534 and a lower surface 1536 of the proximal section 1508 of the body 1506.

In general, the coupler 1526 may have any cross-section that allows for an insertion of the coupler 1526 into the slot 1472 and the cavity 1474 at the distal end 1214 of the cartridge 1204 when the coupler 1526 is in the first orientation, and for a retaining of the coupler 1526 within the slot 1472 and the cavity 1474 at the distal end 1214 of the cartridge 1204 when the coupler 1526 is in the second orientation. As such, the above examples should not be interpreted as limiting on the present disclosure.

In embodiments, the release driver 1504 extends through the locking shaft 1502 and exits proximal to the opening 1230 of the instrument 1206. The release driver 1504 has an expansion bit 1538. The expansion bit 1538 is configured to engage with the cartridge 1204 to cause the cartridge 1204 to release the prosthesis 1202 when the release driver 1504 is actuated, as described in detail further herein.

In some embodiments, at least a portion of the release driver 1504 may be removable from and couplable to the instrument 1206. For example, as illustrated in FIG. 15D, the release driver 1504 includes an interlocking assembly 1540 to couple an actuator portion 1542 (e.g., that includes a shaft with the actuator 1518) with a locking pin 1544 (e.g., that includes the expansion bit 1538 (as a proximal portion), a middle portion 1539, and a distal portion 1541, where the actuator portion 1542 is coupled to the distal portion 1541). In some instances, the interlocking assembly 1540 includes complementary threading. However, any interlocking assembly 1540 is contemplated as being appropriate to disengage the actuator portion 1542 from the locking pin 1544.

In other embodiments, the release driver 1504 may be a single-piece configuration with the actuator 1518 and the expansion bit 1538. The release driver 1504 may be installed within or insertable into the locking shaft 1502. The release driver 1504 may engage the cartridge 1204 once inserted into the opening 1230 of the instrument 1206, including to cause the cartridge 1204 to release the prosthesis 1202 after implantation of the prosthesis 1202.

Where the release driver 1504 is separated into the actuator portion 1542 and the locking pin 1544, it is noted that the locking pin 1544 (with expansion bit 1538) may be provided with an assembly 1634 (e.g., as illustrated in FIGS. 16A-16E) including the cartridge 1204 (and optionally the prosthesis 1202), when the assembly 1634 is inserted into the opening 1230 of the instrument 1206. Alternatively, the actuator portion 1542 and the locking pin 1544 may be provided to the user in a kit as separate components but assembled within the instrument 1206 prior to the assembly 1634 being inserted into the opening 1230 of the instrument 1206.

In embodiments, the middle section 1510 of the body 1506 includes a first portion 1546 proximate to the proximal section 1508 of the body 1506 and at least a second portion 1548 proximate to the distal section 1512 of the body 1506. The first portion 1546 may have a first width or diameter, and the at least a second portion 1548 may have at least a second diameter. For example, the first width or diameter of the first portion 1546 may be less than or equal to a total width between opposite side surfaces of the proximal section 1508, to reduce a need for an increased footprint to accommodate the first portion 1546 at the implantation size where the entire proximal section 1508 is inserted into the patient during implantation. By way of another example, the at least a second width or diameter of the at least a second portion 1548 may be greater than the first width or diameter of the first portion 1546. For instance, the at least a second portion 1548 may be contoured to conform to a user's hand and may include at least a second width or diameter that transitions from the lesser first diameter or diameter of the first portion 1546 to an increased diameter for the user's hand.

It is noted, however, that the at least a second portion 1548 may be able to receive a separate and attachable grip portion that is contoured to conform to a user's hand. In addition, it should be understood that the middle section 1510 may be substantially similar in width or diameter along the entire length of the middle section 1510, without departing from the scope of the present disclosure.

In some embodiments, as illustrated in FIGS. 15B-15E, the middle section 1510 (e.g., in the second portion 1548) includes a lock assembly 1550. The lock assembly 1550 includes a toggle 1552 positioned within at least one slot 1554 in the outer housing 1500. At least one pin 1556 secures the toggle 1552 within the at least one slot 1554 in the outer housing 1500. In one non-limiting example, the toggle 1552 includes an aperture 1558 through which the locking shaft 1502 is inserted, and the toggle 1552 extends through multiple slots 1554 in the outer housing 1500.

The toggle 1552 includes a protrusion 1560 that engages a recess 1562 on the locking shaft 1502 when a biasing element 1564 provides a force on the toggle 1552, locking the locking shaft 1502 and preventing rotation of the locking shaft 1502. For example, rotation of the locking shaft 1502 may be locked after insertion of the cartridge and prosthesis assembly 1634 into the opening 1230 of the instrument 1206. When a user applies a force to the toggle 1552, the at least one biasing element 1564 may be overcome and the toggle 1552 to may disengage from the recess 1562 to allow for rotation of the locking shaft 1502.

In embodiments, the distal section 1512 of the body 1506 includes the actuators 1514, 1516, 1518. For example, the actuator 1514 for the depth stops 1220 is proximal of the actuator 1516 for the locking shaft 1502, and the actuator 1516 for the locking shaft 1502 is proximal of the actuator 1518 for the release driver 1504.

The body 1506 includes a plurality of markers 1566 that indicate the positioning of the depth stops 1220 (e.g., relative to the cartridge 1204 when inserted into the opening 1230 of the instrument 1206) based on a rotation of the actuator 1514. For example, rotation of the actuator 1514 causes an indicator 1568 on the actuator 1514 to align with a particular marker of the plurality of markers 1566. For example, the plurality of markers 1566 may represent a range from 0 to 15 millimeters (mm), and optionally from 0 to 8 mm. By way of another example, the plurality of markers 1566 may be in increments between 0 and 2 mm for a full rotation of the actuator 1514, including optionally 1 mm or 0.5 mm increments for a full rotation of the actuator 1514. It is noted that the markers 1566 and the indicator 1568 may be visual indicators, with an edge of the actuator 1514 and/or a mark (e.g., laser-etched, drawn, an applied sticker, or the like), colors, and/or patterns on the actuator 1514 being the indicator 1568 that aligns with a particular marker 1566. In addition, it is noted that the indicator 1568 may be a window in the actuator 1514 through which the markers 1566 are visible.

As illustrated in FIGS. 15D and 15E, the instrument 1206 may include a tactile indicator 1567 that engages a notch 1569 on the locking shaft 1502. For example, the tactile indicator 1567 may be a mechanical detent housed within a recess 1571 in the body 1506 of the outer housing 1500, which is biased to press against the locking shaft 1502. The tactile indicator 1567 may provide an indication to a user when a full rotation of the actuator 1514 occurs, by engaging with notch 1569 when at the full-rotation position and interfacing with an exterior surface 1573 of the locking shaft 1502 when not at the full-rotation position. This may be beneficial where a line of sight to the visual indicators 1568 is limited or impeded, as a user may still be able to determine when a full rotation of the actuator 1514 (and thus the incremental change in distance that a full rotation of the actuator 1514 represents) occurs. It is noted that the tactile indicator 1567 may be implemented instead of, or in addition to, the markers 1566 and the indicator 1568.

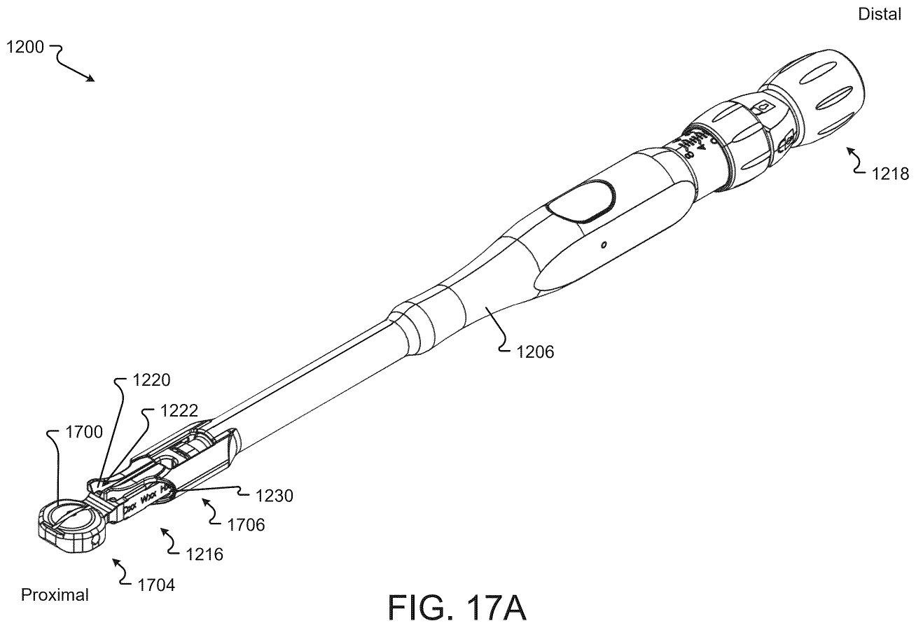

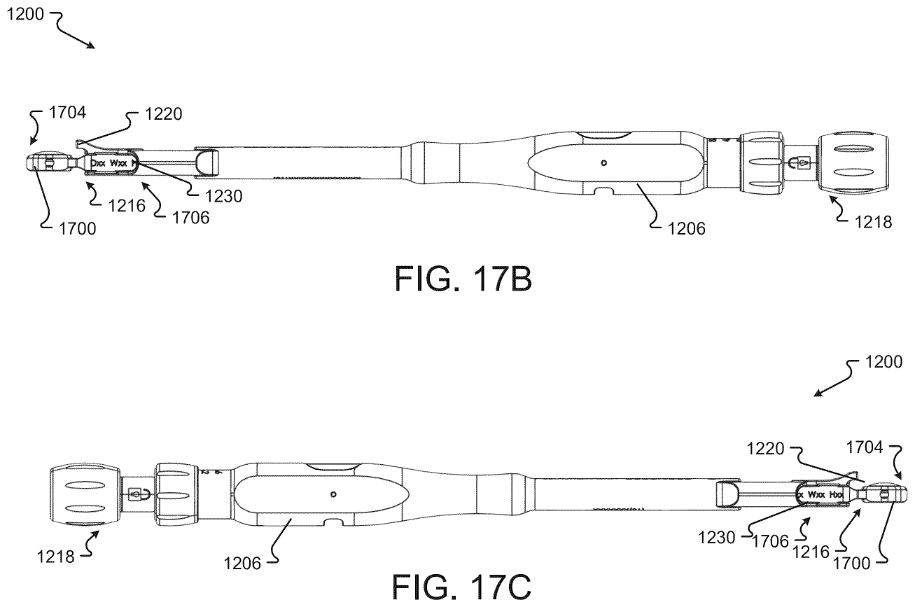

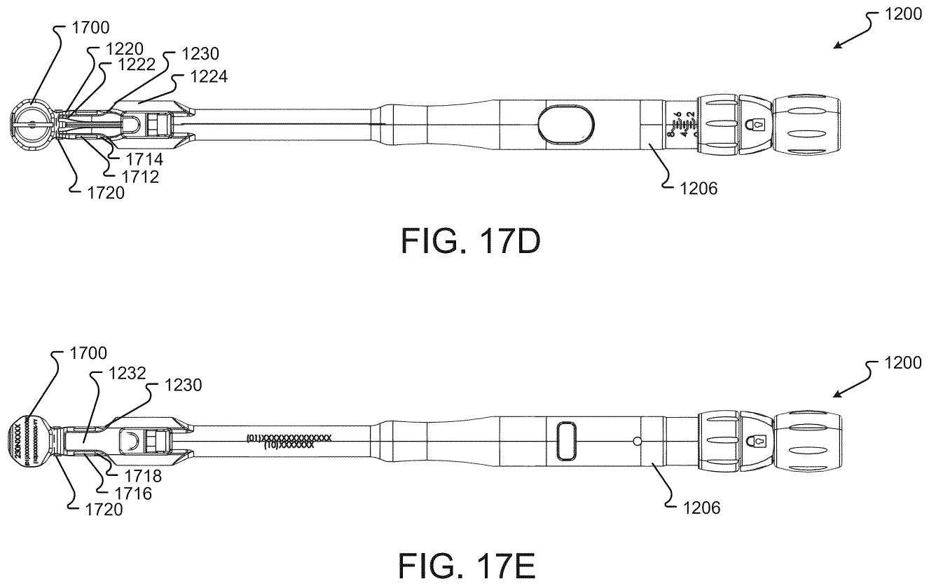

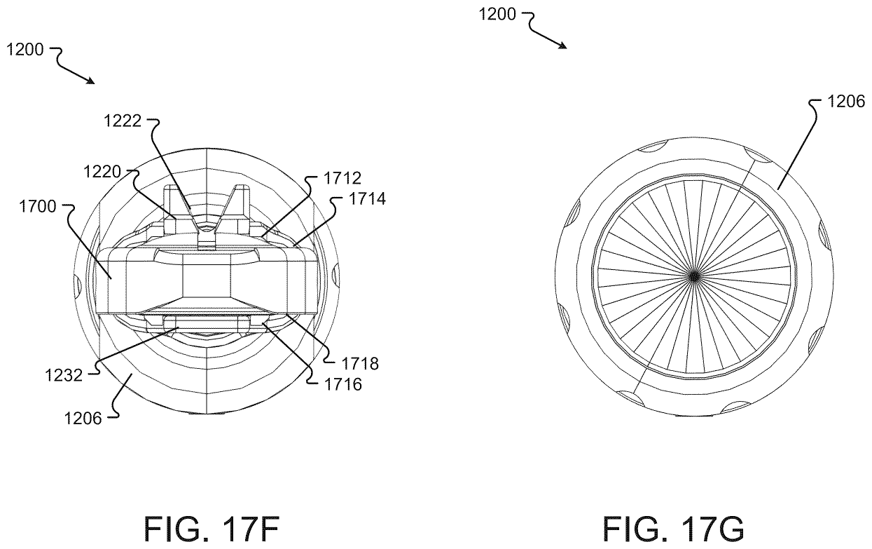

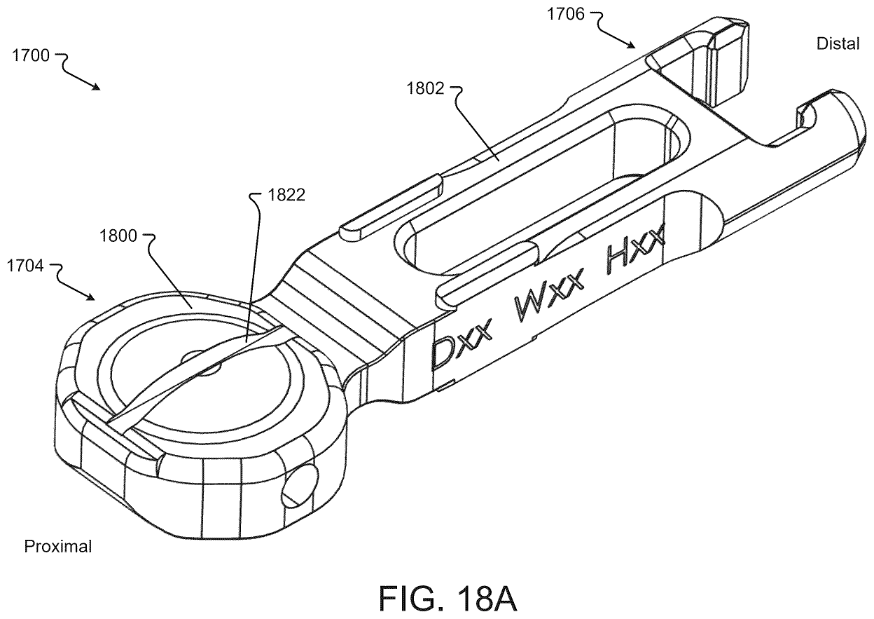

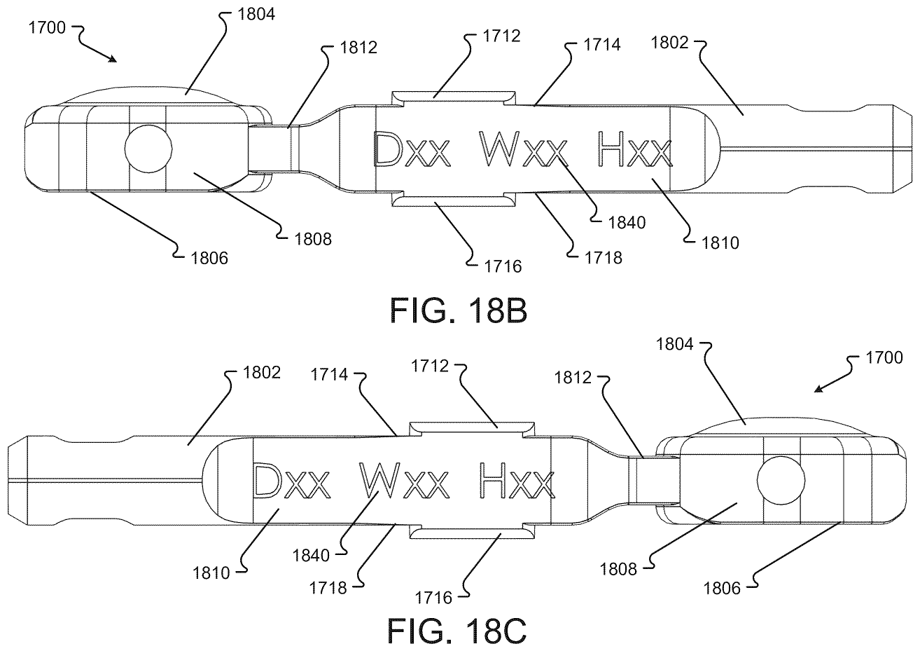

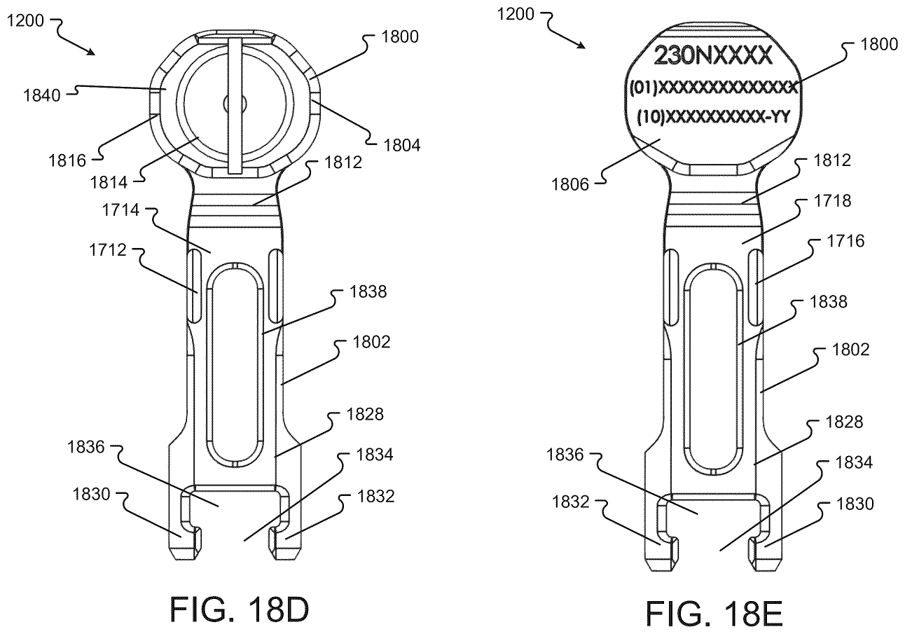

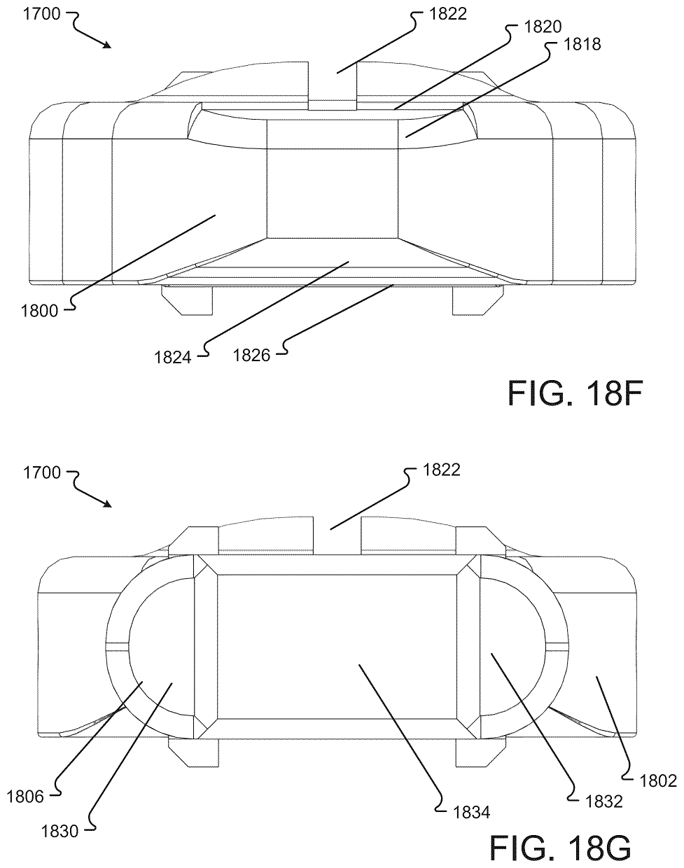

Rotation of the actuator 1514 causes the depth stops 1220 to move by engaging complementary threading 1570 in the actuator 1514 and on the body 1506 of the outer housing 1500. For example, increased engagement between the threading 1570 in the actuator 1514 and on the body 1506 may shorten the length of the outer housing 1500 by pulling the body 1506 into the actuator 1514, while the locking shaft 1502 holds the received cartridge 1204 (or a trial implant 1700, as illustrated in FIGS. 17A-18G and described further herein) in place within the instrument 1206. Doing so sets the adjustment for the depth stop 1220, which is determined by a user based on the depth needed for insertion of the prosthesis 1202 between the adjacent vertebral bodies.

In some embodiments, such as where the release driver 1504 is separated into the actuator portion 1542 and the locking pin 1544, the actuator portion 1542 with the actuator 1518 is removable from, and insertable into, an aperture 1572 in a distal end surface 1574 of the actuator 1516. For example, the user may use the instrument 1206 with only the actuators 1514 and 1516 when inserting the prosthesis 1202 with the cartridge 1204 (e.g., that has the locking pin 1544 inserted within the cartridge 1204). The user may then insert the actuator portion 1542 of the release driver 1504 into the aperture 1572 to engage the locking pin 1544, to cause the cartridge 1204 to release the placed prosthesis 1202. Doing so is beneficial, as it may reduce the possibility the prosthesis is advertently released with the release driver 1504 prior to correct placement at the implantation site.

In embodiments, the actuator portion 1542 includes a proximal member 1575 and a distal shaft 1577. For example, the proximal member 1575 may be coupled to the locking pin 1544, and the distal shaft 1577 may be in communication with the actuator 1518. The proximal member 1575, the distal shaft 1577, and the actuator 1518 may be formed as a single integrated unit. Alternatively, one or more of the proximal member 1575, the distal shaft 1577, and/or the actuator 1518 may be separately formed and coupled to the remainder of the components.

In some configurations, the proximal member 1575 is a smaller diameter than the distal shaft 1577. The graduated diameters may be beneficial to prevent shearing of the actuator portion 1542 when a torque is applied to the actuator 1518, while still allowing for a smaller diameter along the length of the instrument 1206. However, it is contemplated that a diameter of the proximal member 1575 and the distal shaft 1577 may be substantially the same, such that two sections of the actuator portion 1542 may be considered one component, without departing from the scope of the present disclosure.

In embodiments, the actuator 1516 includes at least a first surface 1576 that indicates an unlocked position (or first orientation), and at least a second surface 1578 that indicates a locked position (or second orientation), for the coupler 1526. In embodiments, the actuator 1516 includes a first edge 1580 that is adjacent to a second edge 1582 of the actuator 1514. It is noted that the first surface 1576 and/or the second surface 1578 may include markings (e.g., laser-etched, drawn, an applied sticker, or the like), colors, and/or patterns that provide visual indication of the position of the coupler 1526.

In some configurations, the at least a first surface 1576 and the at least a second surface 1578 may have different contouring. For example, the first surface 1576 may transition into an exterior surface 1579 of the actuator 1514, while the second surface 1578 is offset from the exterior surface 1579 of the actuator 1514. With the contouring of the at least a first surface 1576 and the at least a second surface 1578 of the actuator 1516, the widths of the actuator 1516 at the first edge 1580 and the actuator 1514 at the second edge 1582 are substantially similar or equal on at the at least a first surface 1576, but the width of the actuator 1516 at the first edge 1580 is less than the width of the actuator 1514 at the second edge 1582 on at the at least a second surface 1578. In this regard, a user may observe via a tactile response with the actuator 1516 and/or the actuator 1514 whether the coupler 1526 is in an unlocked or a locked position based on the positioning of the at least a first surface 1576 or the at least a second surface 1578, reducing the possibility of error that the coupler 1526 is prematurely unlocked prior to implantation (or, conversely, reducing the possibility of error that the coupler 1526 remains locks after implantation and during removal of the instrument 1206.

In embodiments, as illustrated in FIGS. 15D-15E and 15I, the distal end 1218 of the instrument 1206 includes a cavity 1584 and an exterior surface 1586. It is noted that the exterior surface 1586 of the actuator 1518 may be substantially smooth, or may include one or more engagement features 1588 (e.g., recesses, protrusions, or the like) that may provide the user with an increased grip to prevent slipping when manipulating the actuator 1518. It is noted that the cavity 1584 may be defined within the actuator 1518 to reduce weight of the instrument 1206. In some non-limiting configurations, the cavity 1584 may additionally be a tool interface. In these configurations, a user may manipulate the actuator 1518 via an exterior surface 1586 and/or by engaging the tool interface. This may be beneficial where additional torque or another force beyond that able to be applied to the exterior surface 1586 by the user is necessary to implant the prosthesis 1202 and/or to actuate the arms 1400, 1402 of the cartridge 1204 to disengage and release the prosthesis 1202, by providing an interface for a tool that is struck (e.g., a punch) during implantation and/or rotated (e.g., a wrench or socket bit, and the like) during disengagement.

FIGS. 16A-16E in general illustrate the engaging of the prosthesis 1202 by the cartridge 1204, and the engaging of the combined prosthesis 1202/cartridge 1204 by the instrument 1206, of the system 1200, in accordance with one or more embodiments of the present disclosure. It should be understood that any embodiment of the prosthesis 1202 illustrated in FIGS. 12A-12G and 13A-13L, the cartridge 1204 illustrated in FIGS. 12A-12G and 14A-14G, and/or the instrument 1206 illustrated in FIGS. 12A-12G and 15A-15I may be directed to the prosthesis 1202, the cartridge 1204 and/or the instrument 1206 illustrated in FIGS. 165A-16H, and vice versa, without departing from the scope of the present disclosure.

In FIG. 16A, the cartridge 1204 (e.g., including, optionally, an installed locking pin 1544 with the expansion bit 1538 of the release driver 1504) is prepared to receive the prosthesis 1202. In FIGS. 16B-16D, the prosthesis 1202 is positioned within the receiver 1416 of the cartridge 1204. It is noted that the optional locking pin 1544 may be inserted within the cartridge 1204 before or after the positioning of the prosthesis 1202 within the receiver 1416 of the cartridge 1204. In embodiments, the prosthesis 1202, the cartridge 1204, and optionally the locking pin 1544 including the expansion bit 1538 of the release driver 1504 is considered an assembly 1634, for purposes of the present disclosure.

In general, as illustrated in FIGS. 16B-16D, the receiver 1416 includes a first edge 1600 with one or more portions 1601 that conforms to the rear edge 1338 of the upper plate 1300, and a second edge 1602 with one or more portions 1603 that conforms to the rear edge 1358 of the lower plate 1302. The edges 1600, 1602 with respective portions 1601, 1603 of the cartridge 1204 and the corresponding edges 1338, 1358 of the prosthesis 1202, in combination of the engagement of the shelves 1426, 1430, 1434, 1438 and the ledges 1340, 1360 of the cartridge 1204 and the prosthesis 1202, creates a more secure connection between the cartridge 1204 and the prosthesis 1202 during implantation.

The portions 1601 of the first edge 1600 of the receiver 1416 that are contoured to conform to the rear edge 1338 of the upper plate 1300, and specifically that are contoured to conform to the engagement portions 1343 along the rear edge 1338, may promote an increased level of securing of the prosthesis 1202 within the cartridge 1204 (e.g., prior to insertion into a patient at an implantation site). Similarly, the portions 1603 of the second edge 1602 of the receiver 1416 that are contoured to conform to the rear edge 1358 of the lower plate 1302, and specifically that are contoured to conform to the engagement portions 1363 along the rear edge 1358, may promote an increased level of securing of the prosthesis 1202 within the cartridge 1204 (e.g., prior to insertion into a patient at an implantation site).

Due to the contouring of the first finger 1422 and the second finger 1424, a side surface 1604 of the first finger 1422 of the first arm 1400 is substantially flush or planar with the side surfaces 1606 and 1608 of the upper plate 1300 and the lower plate 1302, respectively. In addition, a side surface 1610 of the second finger 1424 of the second arm 1402 is substantially flush or planar with opposite side surfaces 1612 and 1614 of the upper plate 1300 and the lower plate 1302, respectively. This reduces the total footprint width of the combined prosthesis 1202 and cartridge 1204 assembly, reducing the width of the implantation site needed during the operation.

Due to the contouring of the shelves 1426, 1430 and the ledges 1340, 1360, the upper surface 1228 of the cartridge 1204 and the superior surface 1306 of the upper plate 1300 are substantially flush or planar. In addition, due to the contouring of the shelves 1434, 1438 and the ledges 1340, 1360, the lower surface 1236 of the cartridge 1204 and the inferior surface 1312 of the lower plate 1302 are substantially flush or planar. This reduces the total footprint height of the combined prosthesis 1202 and cartridge 1204 assembly, reducing the height of the implantation site needed during the operation.

FIGS. 16C and 16D illustrate the engagement of the locking pin 444 with the cartridge 1204. In embodiments, the cartridge 1204 includes an end cavity 1616 that is dimensioned to receive and engage with a proximal end 1618 of the release driver 404 (e.g., a proximal end 1618 of the locking pin 1544, or the expansion bit 1538 of the locking pin 1544). The end cavity 1616 may be positioned within the first arm 1400 and/or within the second arm 1402. In some non-limiting examples, the end cavity 1616 is defined by surfaces in both the first arm 1400 and the second arm 1402. For instance, the first arm 1400 may include a proximal portion 1620 that is proximate to (or is an integrated portion of) the hand 1418 of the first arm 1400, and the second arm 1402 may include a proximal portion 1622 that is proximate to (or is an integrated portion of) the hand 1420 of the second arm 1402. In this instance, interior surfaces defined within the proximal portion 1620 of the first arm 1400 and interior surfaces within the proximal portion 1622 of the second arm 1402 together define the end cavity 1616 within the cartridge 1204.

The end cavity 1616 is dimensioned to receive the proximal end 1618 of the release driver 1504. In some configurations, the positioning of the proximal end 1618 within the end cavity 1616 promotes the cartridge 1204 remaining closed, such that the prosthesis 1202 remains secured within the cartridge 1204 prior to actuation of the release driver 1504.

As illustrated in FIG. 16E, the coupler 1526 of the instrument 1206 is retained within the cavity 1474 and the slot 1472 when the coupler 1526 is in the second orientation. The coupler 1526 is shaped to pass through the slot 1472 into the cavity 1474 in the distal end 1214 of the cartridge 1204 when in a first orientation, and is shaped to be retained within the cavity 1474 when in a second orientation. For example, the orientation of the coupler 1526 may be adjusted through actuation of the actuator 1516. For instance, rotation of the actuator 1516 causes the coupler 1526 to rotate from the first orientation to the second orientation. In this regard, the locking shaft 1502 can receive the slot 1472 and the cavity 1474 at the distal end 1214 of the cartridge 1204 when the cartridge 1204 is inserted into the opening 1230 of the instrument 1206 and then can be rotated to the second orientation to engage the cavity 1474, causing the cartridge 1204 to be retained within the opening 1230 of the instrument 1206. It is noted that the connection of the coupler 1526 of the locking shaft 1502 to the cavity 1474 and the slot 1472 at the distal end 1214 of the cartridge 1204 may be visible through the windows 1522, 1524 of the instrument 1206, but that the visibility is not required for the system 1200 to operate (e.g., as the visibility may not be possible, depending on a depth the proximal end 1216 of the instrument 1206 with the prosthesis 1202 and the cartridge 1204 is inserted into a patient at the implantation site).

In addition, the release driver 1504 is within the cavity 1458 in the cartridge 1204. In some instances, the expansion bit 1538 of the locking pin 1544 of the release driver 1504 is inserted into the cavity 1458 during insertion of the cartridge 1204 into the opening 1230 of the outer housing 1500. In other instances, such as where the locking pin 1544 with the expansion bit 1538 of the release driver 1504 is provided within the cavity 1458 as part of the prosthesis and cartridge assembly 1634, the actuator portion 1542 of the release driver 1504 passes through the locking shaft 1502 and engages the provided locking pin 1544 within the cartridge 1204, after insertion of the prosthesis and cartridge assembly 1634 into the opening 1230 of the outer housing 1500. It is noted that the outer housing 1500 with opening 1230 is not illustrated in FIG. 16C for purposes of clarity only.

Similar to the actuation of the cartridge 104 by the instrument 106 in the system 100, actuation of the release driver 1504 causes the first arm 1400 and the second arm 1402 to expand outward via engagement of the release tabs 1460, 1462 by the expansion bit 1538, as a width between the release tabs 1460, 1462 is less than a width of the expansion bit 1538 (and a width of the end cavity 1616 which initially housed the expansion bit 1538), releasing the prosthesis 1202 once the prosthesis 1202 is inserted into the cavity between the adjacent vertebral bodies. For example, where the actuator portion 1542 connects to the locking pin 1544 via the interlocking assembly 1540, engagement of the components 1542, 1544 via the interlocking assembly 1540 causes the proximal end 1618 including the locking pin 1544 (e.g., with the expansion bit 1538) to be pulled in a distal direction, which moves the expansion bit 1538 within the cavity 1458 such that the proximal end 1618 actuates in the cartridge 1204 from a retracted first position within the end cavity 1616 to an expanded second position in which the proximal end 1618 engages the release tabs 1460, 1462 and expands the cartridge 1204. It is noted that the release tabs 1460, 1462 and the sidewalls of the proximal end 1618 may have complementary contouring to reduce the possibility of binding (and non-actuation of the arms 1400, 1402 in general) when the locking pin 1544 is pulled in the distal direction.

In embodiments, the locking pin 1544 includes an engagement feature 1624 for coupling to portions of the release driver 1504 within the instrument 1206. It is noted that the locking pin 1544 may include similar variations of the engagement feature 1624, such as those shown in FIGS. 5F-5H, without departing from the scope of the present disclosure.

It is noted that release of the prosthesis 1202 by the arms 1400, 1402 of the cartridge 1204 of the assembly 1634 may be understood as being similar to the release shown in FIG. 5F, and that aspects of the description provided above with respect to the components 102, 104, and 444 with 438 of the system 100 are similarly applicable to the components 1202, 1204, and 1544 with 1538 of the system 1200. In some embodiments, the cartridge 1204 includes interlocking components that engage with the release driver 1504 (e.g., on the locking pin 1544 with the expansion bit 1538), such that the release driver 1504 (e.g., the expansion bit 1538) is held within the cartridge 1204 (e.g., during insertion into the opening 1230 of the instrument 1206). For example, the first arm 1400 of the cartridge 1204 includes a locking tab 1626 that engages a first locking recess 1628 on the release driver 1504 (e.g., on the locking pin 1544 with the expansion bit 1538). By way of another example, the second arm 1402 of the cartridge 1204 includes a locking tab 1630 that engages a second locking recess 1632 on the release driver 1504 (e.g., on the locking pin 1544 with the expansion bit 1538).

When the release driver 1504 is actuated via the actuator 1518, the locking recesses 1628, 1632 will disengage from the respective locking tabs 1626, 1630. In some instances, this occurs prior to the proximal end 1618 of the expansion bit 1538 engaging the release tabs 1460, 1462.

It is noted that the cartridge 1204 may only require one of the locking tabs 1626, 1630 and the locking pin 1544 may only require one of the locking recesses 1628, 1632 to hold the locking pin 1544 within the cavity 1458 of the cartridge 1204, without departing from the scope of the present disclosure.

It is noted that portions of the instrument 1206 have been removed from FIGS. 16A-16E for purposes of clarity only.

As illustrated in FIG. 16F, the superior surface 1306 of the upper plate 1300 of the prosthesis 1202 is substantially planar with the upper surface 1448 of the second arm 1402 of the cartridge 1204. Similarly, although not shown in the side view of FIG. 16F, the superior surface 1306 of the upper plate 1300 of the prosthesis 1202 is substantially planar with the upper surface 1446 of the first arm 1400 of the cartridge 1204.

The inferior surface 1312 of the lower plate 1302 of the prosthesis 1202 is substantially planar with the lower surface 1452 of the second arm 1402 of the cartridge 1204. Similarly, although not shown in the side view of FIG. 16F, the inferior surface 1312 of the lower plate 1302 of the prosthesis 1202 is substantially planar with the lower surface 1450 of the first arm 1400 of the cartridge 1204.

With the respective substantially planar surfaces between the prosthesis 1202 and the cartridge 1204, the overall height of the prosthesis and cartridge assembly 1634 is reduced, to reduce the invasiveness of the procedure at the implantation site. This, for example, may be usable for implantation between adjacent vertebral bodies in a spine with normal curvatures (or within a pre-determined range). In this configuration, an overall height of the prosthesis 1202 at the distal end 1210 is substantially equal to an overall height of the cartridge 1204 at the proximal end 1212.

FIG. 16G illustrates a variation of the prosthesis 1202, labeled a prosthesis 1636, in combination with the cartridge 1204 to form a cartridge and prosthesis assembly 1638. Unless otherwise noted, the embodiments of the cartridge and prosthesis assembly 1638 with components 1636, 1204 may be understood as being interchangeable with embodiments of the embodiments of the cartridge and prosthesis assembly 1634 with components 1202, 1204, and vice versa, without departing from the scope of the present disclosure.

The superior surface 1306 of the upper plate 1300 of the prosthesis 1636 is substantially planar with the upper surface 1448 of the second arm 1402 of the cartridge 1204. Similarly, although not shown in the side view of FIG. 16G, the superior surface 1306 of the upper plate 1300 of the prosthesis 1636 is substantially planar with the upper surface 1446 of the first arm 1400 of the cartridge 1204.

However, the inferior surface 1312 of the lower plate 1302 of the prosthesis 1636 is substantially planar with the lower surface 1452 of the second arm 1402 of the cartridge 1204. Similarly, although not shown in the side view of FIG. 16G, the inferior surface 1312 of the lower plate 1302 of the prosthesis 1636 is substantially planar with the lower surface 1450 of the first arm 1400 of the cartridge 1204.

The prosthesis 1636 includes an overall height at the distal end 1210 that is greater than an overall height of the cartridge 1204 at the proximal end 1212. With the increased height of the distal end 1210 of the prosthesis 1636 relative to the proximal end 1212 of the cartridge 1204, the angle between the superior surface 1306 of the upper plate 200 and the inferior surface 1312 of the lower plate 202 differs as compared to the angle between the surfaces 1306, 1312 of the prosthesis 1202. This, for example, may be usable for implantation between adjacent vertebral bodies in a spine with exaggerated or lordotic curvatures (or within a pre-determined range). In this configuration, an overall height of the prosthesis 1202 at the distal end 1210 is substantially equal to an overall height of the cartridge 1204 at the proximal end 1212.

FIGS. 17A-17G in general illustrate the intervertebral disc prosthesis system 1200 with a trial implant 1700, in accordance with one or more embodiments of the present disclosure. It should be understood that any embodiment of the system 1200 illustrated in FIGS. 17A-17G may be directed to the system 1200 illustrated in FIGS. 17A-17G, and vice versa, without departing from the scope of the present disclosure.

The system 1200 in FIGS. 17A-17G includes the trial implant 1700 in place of the prosthesis 1202 and the cartridge 1204, and optionally the osteotome 602 (although not shown). In particular, the instrument 1206 and the trial implant 1700 may first be used to select a corresponding cartridge and prosthesis assembly 1634, and then the trial implant 1700 may be replaced by the cartridge and prosthesis assembly 1634 to implant the prosthesis 1202 with the cartridge 1204. Reference will be made to each of the components 1700, 602 in detail further herein. In addition, any and/or all of the components 1700, 602 may include additional components as described in detail further herein, which are each considered components of the system 1200.

As illustrated in FIGS. 17A-17G, the trial implant 1700 includes a proximal end 1704 and a distal end 1706. The proximal end 1704 of the trial implant 1700 is insertable into a cavity defined between adjacent vertebral bodies. The distal end 1706 of the trial implant 1700 is additionally insertable into the opening 1230 at the proximal end 1216 of the instrument 1206.

Although not shown, the proximal end 608 of the osteotome 602 is insertable into the channel 1222 defined by the at least one depth stop 1220 on the upper tab 1224 of the instrument 1206 (e.g., at the proximal end 1216 of the instrument 1206), similar to the insertion of the proximal end 608 of the osteotome 602 at the proximal end 116 of the instrument 106. For example, the channel 1222 may guide the proximal end 608 of the osteotome 602 to mark a midline on a vertebral body (e.g., an upper vertebral body of the adjacent set between which the prosthesis 1202 is to be implanted). For instance, the distal end 610 of the osteotome 602 may be engaged with by a user (e.g., such as a surgeon or other medical professional) to mark the midline on the vertebral body.

The upper tab 1224 of the instrument 1206 is configured to engage at least one upper wedge 1712 on an upper surface 1714 of the trial implant 1700, to assist in aligning the trial implant 1700 within the opening 1230 in the proximal end 1216 of the instrument 1206. For example, the upper surface 1714 may include two upper wedges 1712 that flank the upper tab 1224 of the instrument 1206 during insertion of the trial implant 1700 into the opening 1230 of the instrument 1206.

In other embodiments, the lower tab 1232 of the instrument 1206 (e.g., at the proximal end 1216 of the instrument 1206) is configured to engage at least one lower wedge 1716 on a lower surface 1718 of the trial implant 1700, to assist in aligning the trial implant 1700 within the opening 1230 in the proximal end 1216 of the instrument 1206. For example, the lower surface 1718 may include two lower wedges 1716 that flank the lower tab 1232 of the instrument 1206 during insertion of the trial implant 1700 into the opening 1230 of the instrument 1206.

It is noted that the trial implant 1700 may include the upper wedges 1712 and/or the lower wedges 1716, either individually or in combination, without departing from the scope of the present disclosure.

Referring back to FIGS. 6D-6E, the surfaces 614, 618 of the trial implant 600 have a width 620 at the location of the wedges 612, 616. As similarly illustrated in FIGS. 17D-17E, the surfaces 1714, 1718 of the trial implant 1700 have a width 1720 at the location of the wedges 1712, 1716. However, a comparison of FIGS. 6D-6E and the interfacing between the instrument 106 and the trial implant 600, versus FIGS. 17D-17E and the interfacing between the instrument 1206 and the trial implant 1700, shows that the width 1720 of the trial implant 1700 is less than the width 620 of the trial implant 600. In particular, the wedges 1712, 1716 may be proximate to sidewalls 1810 of the body 1802 of the trial implant 1700 (e.g., as illustrated in FIGS. 18A-18G), instead of being located on respective superior and inferior surfaces 1804, 1806 of the body 1802 and set a pre-determined distance from the sidewalls 1810 on the respective superior and inferior surfaces 1804, 1806. This reduced width allows for an improved sight line for a user during trialing with the trial implant 1700, as compared to when trialing with the trial implant 600. It is noted that the difference in configuration of the sidewalls 1810 of the body 1802 versus the sidewalls 710 of the body 702 is illustrated in a comparison of FIGS. 18A and 18D-18E, versus FIGS. 7A and 7D-7E.

Although at least a width dimension of the implants 600, 1700 may differ, it should be understood that the trial implants 600, 1700 may be interchangeable with the instruments 106, 1206 of the respective systems 100, 1200, without departing from the scope of the present disclosure.

FIG. 18A-18G in general illustrate the trial implant 1700, in accordance with one or more embodiments of the present disclosure. It should be understood that any embodiment of the trial implant 1700 illustrated in FIGS. 17A-17G may be directed to the trial implant 1700 illustrated in FIGS. 18A-18G, and vice versa, without departing from the scope of the present disclosure.

In embodiments, the trial implant 1700 has a head 1800 at the proximal end 1704. The trial implant 1700 includes a body 1802 at the distal end 1706.

In embodiments, the head 1800 is a static size with a pre-defined depth (Dxx), width (Wxx), and/or height (Hxx). In other embodiments, the head 1800 is adjustable in one or more of depth, width, and/or height (e.g., via actuation of the release driver 1504 following the securing of the trial implant 1700 within the opening 1230 of the instrument 1206 via the locking shaft 1502). In general, the trial implant 1700 is configured to approximate the dimensions of the cartridge 1204 and a particular prosthesis 1202 having a pre-defined depth, width, and/or height, so as to correctly interface with the instrument 1206 and so as to correctly size a cavity between adjacent vertebral bodies for the particular prosthesis 1202.

In embodiments, the head 1800 includes a superior surface 1804, an inferior surface 1806, and one or more sidewalls 1808. The body 1802 includes the upper surface 1714 with the upper wedges 1712, the lower surface 1718 with the lower wedges 1716, and one or more sidewalls 1810.

In embodiments, the head 1800 and the body 1802 are connected via a transition region 1812. For example, at least a portion of the transition region 1812 may be substantially equal to a width of the head 1800 and/or the body 1802. By way of another example, at least a portion of the transition region 1812 may be narrower in width than the head 1800 and/or the body 1802. It is noted that the head 1800 and the body 1802 may be a single integrated component. Alternatively, the head 1800 and the body 1802 may be coupled together (e.g., at the transition region 1812), such that the head 1800 is interchangeable with another head 1800 on the body 1802, without departing from the scope of the present disclosure.

In embodiments, the superior surface 1804 of the head 1800 includes a dome region. In general, the dome region is shaped to contour to conform to a dome region of a particular prosthesis 1202, which is optionally convex and dome-shaped on the superior surface 1306 of the upper plate 1300 of a particular prosthesis 1202. For instance, the superior surface 1804 of the trial implant 1700 may be contoured to conform to a substantially spherical portion for the convex dome shape.

For example, the dome region may include a convex portion 1814 at least partially surrounded by a substantially flat or planar portion 1816 in the superior surface 1804. The transition between the convex portion 1814 and the substantially flat or planar portion 1816 may be sloped and/or without a step-like change in height. Alternatively, the transition between the convex portion 1814 and the substantially flat or planar portion 1816 may have a step-like change in height.

In embodiments, the head 1800 includes a chamfer 1818 (or rounded portion) on a front edge or leading edge 1820 of the superior surface 1804. For example, the chamfer 1818 may facilitate insertion of the trial implant 1700 into the cavity defined between the adjacent vertebral bodies during trialing.

In embodiments, the superior surface 1804 of the head 1800 includes an alignment feature 1822 (e.g., within the convex portion 1814) that optionally aligns with complementary features on the instrument 1206. For example, the alignment feature 1822 may be a groove, a recess, a protrusion, a mark (e.g., laser-etched, drawn, an applied sticker, or the like), colors, and/or patterns or another feature that aligns with complementary alignment features on the instrument 1206 (e.g., such as the channel 1222). For instance, the alignment feature 1520 and/or the alignment feature 1822 may correspond to a midline of the adjacent vertebral bodies (e.g., as created by the osteotome 602) between which a prosthesis 1202 is being implanted. In this regard, a user (e.g., a surgeon, or other medical professional) may utilize the combination of the alignment feature 1822 and the complementary features on the instrument 1206 to sight the trial implant 1700 during insertion into the cavity defined between the adjacent vertebral bodies at the implantation site during trialing.

In embodiments, the inferior surface 1806 is substantially flat or planar. For example, the inferior surface 1806 of the trial implant 1700 may be contoured to conform to a substantially flat or planar portion of the inferior surface 1312 of the lower plate 1302 of a particular prosthesis 1202.

In embodiments, the head 1800 includes a chamfer 1824 (or rounded portion) on a front edge or leading edge 1826 of the inferior surface 1806. For example, the chamfer 1824 may facilitate insertion of the trial implant 1700 into the cavity defined between the adjacent vertebral bodies during trialing.

It is contemplated that the superior surface 1804 and the inferior surface 1806 may be configured to replicate an exemplary natural state for the prosthesis 1202, where the upper plate 1300 is offset slightly forward of, and tilted slightly toward, the lower plate 1302. For example, the superior surface 1804 may be offset from the inferior surface 1806 by approximately 0.5 mm. In addition, the superior surface 1804 may be angled toward the inferior surface 1806 by approximately 3°. It is noted, however, that this is only an exemplary state, and that other natural states are contemplated, without departing from the scope of the present disclosure.

In embodiments, the body 1802 of the trial implant 1700 includes a crossmember 1828. In some embodiments, the crossmember 1828 is a thickness that is less than a total thickness of the trial implant 1700. This allows the body 1802 of the trial implant 1700 to be inserted into the opening 1230 at the proximal end 1216 of the instrument 1206 when the locking shaft 1502 is in a first orientation and may be secured within the opening 1230 when the locking shaft 1502 in a second orientation. Thus, as illustrated in FIGS. 18A-18G, in some embodiments the upper surface 1714 and the lower surface 1718 of the trial implant 1700 are substantially flat or planar along the entire length of the body 1802, aside from the wedges 1712, 1716 that extend from the respective surfaces 1714, 1718.

The body 1802 additionally includes a first tail 1830 and a second tail 1832 at a distal end 1706 of the trial implant 1700. The space between the first tail 1830 and the second tail 1832 defines a slot 1834 of the distal end 1706 of the trial implant 1700. In addition, the crossmember 1828 and the tails 1830, 1832 define a cavity 1836 of the distal end 1706 of the trial implant 1700.

In some embodiments, the cavity 1836 has a greater width than the slot 1834. As with the cartridge 1204, the coupler 1526 of the locking shaft 1502 of the instrument 1206 may be inserted within the slot 1834 when in a first orientation and re-oriented within the cavity 1836 to a second orientation, causing the coupler 1526 of the instrument 1206 to be retained within the cavity 1836 by the slot 1834 when the coupler 1526 is in the second orientation.

In embodiments, the body 1802 optionally includes a cavity 1838 between the head 1800 and the crossmember 1828. For example, the cavity 1838 may be beneficial for weight reduction of the trial implant 1700, so as to more closely emulate an assembly 1634 of a cartridge 1204 and a particular prosthesis 1202.

In embodiments, a surface of the trial implant 1700 includes a plurality of indicators 1840. The plurality of indicators 1840 may be marks (e.g., laser-etched, drawn, an applied sticker, or the like), colors, and/or patterns that are on the surface. For example, the surface may be the sidewalls 1810 of the body 1802, so that the plurality of indicators 1840 are more easily applied to a larger, substantially planar surface of the trial implant 1700 (i.e., as compared to being applied to the superior surface 704 of the head 700 of the trial implant 700). In general, the plurality of indicators 1840 may include information about the trial implant 1700, to assist in the selection of the corresponding prosthesis 1202 (and/or corresponding cartridge and prosthesis assembly 1634). In some instances, the plurality of indicators 1840 may include, but are not limited to, information about the depth (Dxx), width (Wxx), and/or height (Hxx) of the head 1800.

It is contemplated that the body 1506 may include an opening between the cavities 1836 and 1838. For example, the opening may allow for passage of the locking pin 1544, including where the locking pin 1544 is installed in the instrument 1206 and/or is integrated with the release driver 1504, as opposed to being provided with the cartridge and prosthesis assembly 1634.

FIGS. 19A-19H in general illustrate a variation of the prosthesis 1202 of the system 1200, labeled a prosthesis 1902, in accordance with one or more embodiments of the present disclosure. It should be understood that any embodiment of the prosthesis 1902 illustrated in FIGS. 19A-19H may be directed to the prosthesis 1202 illustrated in FIGS. 12A-12G, FIGS. 13A-13L, and/or FIGS. 16A-16G, and vice versa, without departing from the scope of the present disclosure unless otherwise noted.

It should be understood that the prosthesis 1902 is usable with the process 900 in place of the prosthesis 1202, including with the cartridge 1204 and the instrument 1206, without departing from the scope of the present disclosure.

In embodiments, the prosthesis 1902 includes the upper plate 1300, the lower plate 1302, and the insert 1304. In general, the upper plate 1300 and the lower plate 1302 of the prosthesis 1902 are similar to the upper plate 1300 and the lower plate 1302 of the prosthesis 1202. In particular, a dome region 1917 of the upper plate 1300 includes a concave portion 1922 at least partially surrounded by a substantially flat or planar portion 1924 in the inferior surface 1308. The transition between the concave portion 1922 and the substantially flat or planar portion 1924 may have a step-like change in height. For example, the step-like change in height may be formed by a substantially flat or rounded rim 1926 that extends away from (e.g., downward) from the inferior surface 1908.

However, as illustrated in a comparison of FIGS. 13D and 19D, the concave portion 1922 of the upper plate 1300 of the prosthesis 1902 is smaller than the concave portion 1322 of the upper plate 1300 of the prosthesis 1202, and the rim 1926 of the upper plate 1300 of the prosthesis 1902 is larger than the rim 1326 of the upper plate 1300 of the prosthesis 1202. This difference in size is to interface with a ring or flange portion 1969 that surrounds the convex region 1968 of the superior surface 1314 of the insert 1304 of the prosthesis 1902.

In general, the interfacing of the prosthesis 1902 between the rim 1926 and the ring 1969 when the upper plate 1300 moves relative to the insert 1304 provides a limit or stop on the motion of the prosthesis 1902, as compared to the insert 1304 of the prosthesis 1202 with only the convex region 1368. This limit or stop reduces the possibility of over-extension of the upper plate 1300 (or over-compression of the insert 1304) during move of the adjacent vertebral bodies when the prosthesis 1902 is implanted, preventing excessive damage to the components 1300, 1302, 1304 and/or slippage of the prosthesis 1902 when implanted between the adjacent vertebral bodies.

FIGS. 19E-19H illustrate four positions of the upper plate 1300 relative to the insert 1304 (and the lower plate 1302) when the rim 1926 interfaces with the ring 1969. In embodiments, the ring 1969 is set at an angle 1975. For example, the ring 1969 may be angled with respect to the inferior surface 1316 of the insert 1304, with the transition or edge between the ring 1969 and the concave portion 1922 being at an increased height as compared to the transition or edge between the ring 1969 and the sidewalls 1371 / endwalls 1374. The angle 1975 may be included to limit flexion of the spine in the index level where the prosthesis 1902 is implanted. However, it is contemplated that the ring 1969 may be substantially parallel with the inferior surface 1316 of the insert 1304, without departing from the scope of the present disclosure.

With the inclusion of the at least one protrusion 1348 and corresponding recesses 1370, the insert 1304 of the prosthesis 1902 has an approximately circular cross-section when viewed from the superior surface 1314, or generally has a cross-section with a major and minor axis that are approximately the same. In contrast, the insert 1304 of the prosthesis 1202 has a cross-section with a longer major axis (e.g., across the width of the prosthesis 1202) and a shorter minor axis (e.g., along the length or direction of insertion of the prosthesis 1202). However, the major and minor axes of the insert 1304 of the prosthesis 1902 are closer in dimension to allow for interfacing between the rim 1926 and the ring 1969. The approximately similar major and minor axes of the insert 1304 of the prosthesis 1902 are beneficial, as it provides for a more consistent interfacing between the rim 1926 and the ring 1969 during movement by the user regardless of the orientation of the adjacent vertebral bodies between which the prosthesis 1902 is implanted.

Embodiments of the present disclosure are directed to the inferior surface 1308 of the upper plate 1300 including a concave portion 1922, a substantially flat or planar portion 1924, and a protruding rim 1926 that may interface with a ring 1969 on the insert 1304. However, it should be understood that the concave portion 1922 may transition directly into the substantially flat or planar portion 1924 such that the ring 1969 interfaces with the substantially flat or planar portion 1924, and without need for the separating rim 1926, without departing from the scope of the present disclosure.

As illustrated in FIGS. 19A-19H, the interfacing rim 1926 and ring 1969 may be usable with the multi-protrusion configuration, with the superior surface 1310 of the lower plate 1302 including at least one protrusion 1348 and the insert 1304 including at least one corresponding groove 1372. However, as illustrated in FIGS. 20A-20H, the interfacing rim 1926 and ring 1969 may also be used on an insert 1304 with a central recess 2070 that receives a central protrusion 2048 on the lower plate 1302.

FIGS. 20A-20H in general illustrate a variation of the prosthesis 1202 of the system 1200, labeled a prosthesis 2002, in accordance with one or more embodiments of the present disclosure. It should be understood that any embodiment of the prosthesis 2002 illustrated in FIGS. 20A-20H may be directed to the prosthesis 1202 illustrated in FIGS. 12A-12G, FIGS. 13A-13L, and/or FIGS. 16A-16G, and/or the prosthesis 1902 illustrated in FIGS. 19A-19H, and vice versa, without departing from the scope of the present disclosure unless otherwise noted.

It should be understood that the prosthesis 2002 is usable with the process 900 in place of the prosthesis 1202, including with the cartridge 1204 and the instrument 1206, without departing from the scope of the present disclosure.

In embodiments, the prosthesis 1902 includes the upper plate 1300, the lower plate 1302, and the insert 1304. In general, the upper plate 1300 and the lower plate 1302 of the prosthesis 1902 are similar to the upper plate 1300 and the lower plate 1302 of the prosthesis 1202. However, the lower plate 1302 of the prosthesis 2002 includes a central protrusion 2048 that is received by a recess 2070 within the insert 1304, similar to the prosthesis 102 of the system 100, instead of the multiple protrusions 1348 that have corresponding recesses 1370 as previously described herein.

In addition, a dome region 2017 of the upper plate 1300 includes a concave portion 2022 at least partially surrounded by a substantially flat or planar portion 2024 in the inferior surface 1308. The transition between the concave portion 2022 and the substantially flat or planar portion 2024 may have a step-like change in height. For example, the step-like change in height may be formed by a substantially flat or rounded rim 2026 that extends away from (e.g., downward) from the inferior surface 2008.

However, as illustrated in a comparison of FIGS. 13D and 20D, the concave portion 2022 of the upper plate 1300 of the prosthesis 2002 is smaller than the concave portion 1322 of the upper plate 1300 of the prosthesis 1202, and the rim 2026 of the upper plate 1300 of the prosthesis 2002 is larger than the rim 1326 of the upper plate 1300 of the prosthesis 1202. This difference in size is to interface with a ring or flange portion 2069 that surrounds the convex region 2068 of the superior surface 1314 of the insert 1304 of the prosthesis 2002.

In general, the interfacing of the prosthesis 2002 between the rim 2026 and the ring 2069 when the upper plate 1300 moves relative to the insert 1304 provides a limit or stop on the motion of the prosthesis 2002, as compared to the insert 1304 of the prosthesis 1202 with only the convex region 1368. This limit or stop reduces the possibility of over-extension of the upper plate 1300 (or over-compression of the insert 1304) during move of the adjacent vertebral bodies when the prosthesis 2002 is implanted, preventing excessive damage to the components 1300, 1302, 1304 and/or slippage of the prosthesis 2002 when implanted between the adjacent vertebral bodies.

FIGS. 20E-20H illustrate four positions of the upper plate 1300 relative to the insert 1304 (and the lower plate 1302) when the rim 2026 interfaces with the ring 2069. In embodiments, the ring 2069 is set at an angle 2075. For example, the ring 2069 may be angled with respect to the inferior surface 1316 of the insert 1304, with the transition or edge between the ring 2069 and the concave portion 2022 being at an increased height as compared to the transition or edge between the ring 2069 and the endwalls 1374. The angle 2075 may be included to limit flexion of the spine in the index level where the prosthesis 2002 is implanted. However, it is contemplated that the ring 2069 may be substantially parallel with the inferior surface 1316 of the insert 1304, without departing from the scope of the present disclosure.

With the inclusion of the centrally-located protrusion 2048 and the recess 2070, the insert 1304 of the prosthesis 2002 has a substantially circular cross-section when viewed from the superior surface 1314, or generally has a cross-section with a major and minor axis that are substantially the same. In contrast, the insert 1304 of the prosthesis 1202 has a cross-section with a longer major axis (e.g., across the width of the prosthesis 1202) and a shorter minor axis (e.g., along the length or direction of insertion of the prosthesis 1202). The substantially similar major and minor axes of the insert 1304 of the prosthesis 2002 are beneficial, as it provides for a more consistent interfacing between the rim 2026 and the ring 2069 during movement by the user regardless of the orientation of the adjacent vertebral bodies between which the prosthesis 2002 is implanted.

Embodiments of the present disclosure are directed to the inferior surface 1308 of the upper plate 1300 including a concave portion 2022, a substantially flat or planar portion 2024, and a protruding rim 2026 that may interface with a ring 2069 on the insert 1304. However, it should be understood that the concave portion 2022 may transition directly into the substantially flat or planar portion 2024 such that the ring 2069 interfaces with the substantially flat or planar portion 2024, and without need for the separating ring 2069, without departing from the scope of the present disclosure.

It is contemplated that the prosthesis 1902 and/or 2002 may include the grooves 1372 in the inferior surface 1316 of the insert 1304, without departing from the scope of the present disclosure.

In this regard, advantages of the present disclosure include, but are not limited to, an intervertebral disc prosthesis, and systems and methods for implantation of the same. In particular, advantages of the present disclosure are directed to the prosthesis that is insertable between adjacent vertebral bodies, and that includes an upper plate, a lower plate, and an insert enclosed therebetween. The insert is able to move within a cavity or space defined between the upper and lower plates to allow for repositioning during movement of the patient in which the prosthesis is implanted, but is also secured by features of the upper and/or lower plates to prevent slippage or loss of the insert from between the upper and lower plates when implanted.

Advantages of the present disclosure also include, but are not limited to, an instrument able to interface with a cartridge, where the cartridge is able to engage the prosthesis. Following engagement of the prosthesis by the cartridge to form a cartridge and prosthesis assembly, the instrument is able to interface with the cartridge via insertion of the cartridge into an outer housing of the instrument and actuation of a locking shaft of the instrument. The instrument can then be used to position the prosthesis within the implantation site. Once the prosthesis is positioned with a cavity defined between adjacent vertebral bodies, actuation of a release driver within the instrument causes the cartridge to disengage from the prosthesis, allowing for the removal of the cartridge and the instrument from the implantation site.

Advantages of the present disclosure also include, but are not limited to, trial implants and an osteotome for use during trialing and prior to implantation of the prosthesis.

While various embodiments of the present disclosure have been described in detail, it is apparent that modifications and alterations of those embodiments will occur to those skilled in the art. However, it is to be understood that such modifications and alterations are within the scope and spirit of the present disclosure, as set forth in the following claims. Further, the disclosure described herein is capable of other embodiments and of being practiced or of being carried out in various ways. It is to be understood that the phraseology and terminology used herein is for the purpose of description and should not be regarded as limiting.

What is claimed is:

1. A prosthesis for an intervertebral disc prosthesis system, the prosthesis comprising:
an upper plate including a superior surface with at least one set of upper teeth, and an inferior surface;
a lower plate including a superior surface with at least one protrusion, and an inferior surface with at least one set of lower teeth; and
an insert located between the upper plate and the lower plate, wherein the insert includes a superior surface with a convex region, wherein the insert includes an inferior surface that is substantially flat, wherein the insert includes at least one recess able to receive the at least one protrusion, wherein a particular recess of the at least one recess corresponds to a particular protrusion of the at least one protrusion,
wherein at least one of a rear edge of the upper plate and a rear edge of the lower plate includes at least one engagement feature able to engage with a corresponding engagement feature of a cartridge that is insertable into an instrument.

2. The prosthesis of claim 1, wherein the at least one set of upper teeth of the upper plate includes a first set of upper teeth proximate to a first side edge of the upper plate, and a second set of upper teeth proximate to a second side edge of the upper plate.

3. The prosthesis of claim 2, wherein the at least one engagement feature is on the upper plate, wherein the first set of upper teeth of the upper plate at least partially align with a first engagement feature of the at least one engagement feature on the upper plate, and wherein the second set of upper teeth of the upper plate at least partially align with a second engagement feature of the at least one engagement feature on the upper plate.

4. The prosthesis of claim 1, wherein the upper plate includes one or more alignment teeth located along a midline of the upper plate.

5. The prosthesis of claim 4, wherein the one or more alignment teeth are located on a convex region of the superior surface of the upper plate, and wherein the at least one set of upper teeth includes a first set of upper teeth and a second set of upper teeth that flank the convex region of the superior surface of the upper plate.

6. The prosthesis of claim 1, wherein the inferior surface of the upper plate includes a concave portion that is contoured to conform to the convex region of the superior surface of the insert.

7. The prosthesis of claim 6, wherein the concave portion of the inferior surface of the upper plate is at least partially surrounded by a substantially flat portion of the inferior surface of the upper plate.

8. The prosthesis of claim 7, wherein the concave portion of the inferior surface of the upper plate is separated from the substantially flat portion of the inferior surface of the upper plate by a rim that extends from the inferior surface.

9. The prosthesis of claim 8, wherein the convex region of the superior surface of the insert is at least partially surrounded by a ring, and wherein the ring is able to engage with the rim extending from the inferior surface of the upper plate.

10. The prosthesis of claim 1, wherein the at least one set of lower teeth of the lower plate includes a first set of lower teeth proximate to a first side edge of the lower plate, and a second set of lower teeth proximate to a second side edge of the lower plate.

11. The prosthesis of claim 10, wherein the at least one engagement feature is on the lower plate, wherein the first set of lower teeth of the lower plate is located between a midline of the lower plate and a first engagement feature of the at least one engagement feature on the lower plate, and wherein the second set of lower teeth of the lower plate is positioned between the midline of the lower plate and a second engagement feature of the at least one engagement feature on the lower plate.

12. The prosthesis of claim 1, wherein the lower plate includes one or more alignment teeth located along a midline of the lower plate.

13. The prosthesis of claim 1, wherein the at least one protrusion includes a single protrusion located on a midline of the lower plate, and wherein the at least one recess includes a single recess that is located on a midline of the insert and extends a selected depth into the inferior surface of the insert.

14. The prosthesis of claim 1, wherein the at least one protrusion includes a first protrusion located proximate to a first side edge of the lower plate, and a second protrusion located proximate to a second side edge of the lower plate, and
wherein the at least one recess includes a first recess corresponding to the first protrusion and that intersects a first sidewall of the insert while extending through the insert from the inferior surface of the insert to the superior surface of the insert, and a second recess corresponding to the second protrusion and that intersects a second sidewall of the insert while extending through the insert from the inferior surface of the insert to the superior surface of the insert.

15. The prosthesis of claim 1, wherein the at least one engagement feature is on the upper plate, and wherein the at least one engagement feature includes a ledge that extends from the rear edge of the upper plate to a notch in the upper plate.

16. The prosthesis of claim 15, wherein the ledge transitions directly into the notch such that a shared surface of the ledge and the notch is at least partially defined by an edge with a straight portion.

17. The prosthesis of claim 1, wherein the at least one engagement feature is on the lower plate, and wherein the at least one engagement feature includes a ledge that extends from the rear edge of the lower plate to a notch in the lower plate.

18. The prosthesis of claim 17, wherein the ledge transitions directly into the notch such that a shared surface of the ledge and the notch is at least partially defined by an edge with a straight portion.

19. A prosthesis for an intervertebral disc prosthesis system, the prosthesis comprising:
an upper plate including a superior surface with at least one set of upper teeth, and an inferior surface;
a lower plate including a superior surface with a first protrusion located proximate to a first side edge of the lower plate and a second protrusion located proximate to a second side edge of the lower plate, and an inferior surface with at least one set of lower teeth; and
an insert located between the upper plate and the lower plate, wherein the insert includes a superior surface with a convex region, wherein the insert includes an inferior surface that is substantially flat, wherein the insert includes a first recess corresponding to the first protrusion and that intersects a first sidewall of the insert while extending through the insert from the inferior surface of the insert to the superior surface of the insert, wherein the insert includes a second recess corresponding to the second protrusion and that intersects a second sidewall of the insert while extending through the insert from the inferior surface of the insert to the superior surface of the insert,
wherein at least one of a rear edge of the upper plate and a rear edge of the lower plate includes at least one engagement feature able to engage with a corresponding engagement feature of a cartridge insertable into an instrument of the intervertebral disc prosthesis system.

20. A prosthesis for an intervertebral disc prosthesis system, the prosthesis comprising:
an upper plate including a superior surface with at least one set of upper teeth, and an inferior surface, wherein the upper plate includes at least one engagement feature with a ledge that extends from a rear edge of the upper plate to a notch in the upper plate, wherein the ledge of the upper plate transitions directly into the notch of the upper plate such that a shared surface of the ledge and the notch of the upper plate is at least partially defined by an edge with a straight portion of the upper plate;
a lower plate including a superior surface with at least one protrusion, and an inferior surface with at least one set of lower teeth, wherein the lower plate includes at least one engagement feature with a ledge that extends from a rear edge of the lower plate to a notch in the lower plate, wherein the ledge of the lower plate transitions directly into the notch of the lower plate such that a shared surface of the ledge and the notch of the lower plate is at least partially defined by an edge with a straight portion of the lower plate; and
an insert located between the upper plate and the lower plate, wherein the insert includes a superior surface with a convex region, wherein the insert includes an inferior surface that is substantially flat, wherein the insert includes at least one recess able to receive the protrusion, wherein a particular recess of the at least one recess corresponds to a particular protrusion of the at least one protrusion.
